(19) United States
(12) Patent Application Publication
(10) Pub. No.: US 2016/0362478 A1
(43) Pub. Date: Dec. 15, 2016

(54) CD4-MIMETIC SMALL MOLECULES SENSITIZE HUMAN IMMUNODEFICIENCY VIRUS TO VACCINE-ELICITED ANTIBODIES

(71) Applicants: Navid MADANI, Westboro, MA (US); Amy M. PRINCIOTTO, Attleboro, MA (US); Judith M. LALONDE, Havertown, PA (US); Richard T. WYATT, San Diego, CA (US); Jong Woo PARK, Daejeon (KR); DANA-FARBER CANCER INSTITUTE INC., Boston, MA (US); THE JOHNS HOPKINS UNIVERSITY, Baltimore, MD (US); THE TRUSTEES OF THE UNIVERSITY OF PENNSYLVANIA, Philadelphia, PA (US)

(72) Inventors: Joseph SODROSKI, Medford, MA (US); Navid MADANI, Newton, MA (US); Amy M. PRINCIOTTO, Attleboro, MA (US); Arne SCHON, Baltimore, MD (US); Judith M. LaLONDE, Haverton, PA (US); Ernesto FREIRE, Baltimore, MD (US); Amos B. SMITH, Merion, PA (US); Richard T. WYATT, San Diego, CA (US); Jongwoo PARK, Daejeon (KR); Joel R. COURTER, Glen Mills, PA (US); David M. JONES, Zionsville, IN (US); Wayne A. HENDRICKSON, New York, NY (US); Xueling WU, Potomac, MD (US); Matthew LE-KHAC, Brooklyn, NY (US); Peter D. KWONG, Washington, DC (US); Young Do KWON, Kensington, MD (US); John R. MASCOLA, Rockville, MD (US)

(21) Appl. No.: 15/117,866

(22) PCT Filed: Feb. 10, 2015
(86) PCT No.: PCT/US2015/015182

§ 371 Date: Aug. 10, 2016

Related U.S. Application Data
(60) Provisional application No. 61/937,868, filed on Feb. 10, 2014.

(51) Int. Cl. C07K 16/10; A61K 31/438; A61K 31/4468; A61K 39/42; A61K 31/167

(52) U.S. Cl. CPC C07K 16/1063; A61K 39/42; A61K 31/167; A61K 31/4468; A61K 31/438; A61K 2039/505

(57) ABSTRACT

Described herein are methods of generating a protein binding domain that specifically binds to gp120 in a specific conformational state, comprising contacting gp120 with a CD4-mimetic compound, thereby forming gp120 in the specific conformational state; and generating antibodies to gp120 in the specific conformation state. Relatedly, the disclosure also describes methods of neutralizing HIV-1, comprising contacting HIV-1 with an effective amount of a CD4-mimetic compound, thereby forming HIV-1 having gp120 in a specific conformational state; and contacting the HIV-1 in the specific conformational state with an antibody.

### RELATED APPLICATIONS

This application claims the benefit of priority to U.S. Provisional Patent Application Ser. No. 61/937,868, filed Feb. 10, 2014, which is hereby incorporated by reference.

### GOVERNMENT SUPPORT

This invention was made with government support under Grants GM56550, AI100645, AI24755, and AI090682 awarded by the National Institutes of Health, and Grant MCB-1157506 awarded by the National Science Foundation. The government has certain rights in the invention. This statement is included solely to comply with 37 C.F.R. §401.14(a)(f)(4) and should not be taken as an assertion or admission that the application discloses and/or claims only one invention.

### BACKGROUND OF THE DISCLOSURE

Preventing sexual transmission of human immunodeficiency virus (HIV-1) is critical for altering the course of the global pandemic of acquired immunodeficiency syndrome (AIDS). Currently, approximately 34 million people are living with HIV-1 infection; 2.5 million people are newly infected with the virus annually, and nearly 1.7 million individuals succumb each year to AIDS. Hence, there is an urgent need to develop vaccines or other strategies that can prevent HIV-1 transmission.

HIV-1-neutralizing antibodies are an important component of a protective vaccine-induced immune response. Passive administration of HIV-1-neutralizing antibodies protects monkeys from intravenous and mucosal challenge with simian-human immunodeficiency viruses (SHIVs). The trimeric envelope glycoprotein (Env) spike on the virion surface is the only HIV-1-specific target accessible to neutralizing antibodies. The presence of circulating antibodies against a specific region of Env (the gp120 V2 variable region) correlated with the partial protection seen in the RV 144 clinical vaccine trial. Thus, the generation of anti-Env antibodies, particularly neutralizing antibodies, may be critical for a successful HIV-1 vaccine.

The HIV-1 Env spike, which is composed of three gp120 exterior Envs and three gp41 transmembrane Envs, mediates virus entry into host cells. The unliganded HIV-1 Env is metastable. Binding of gp120 to the initial receptor, CD4, triggers Env conformational changes that result in the formation/exposure of two elements: 1) the gp120 binding site for the second receptor, CCR5 or CXCR4, and 2) the gp41 heptad repeat (HR1) coiled coil. Binding of gp120 to the CCR5 or CXCR4 coreceptor is thought to induce further Env conformational changes that result in the formation of an energetically stable gp41 six-helix bundle that promotes the fusion of the viral and target cell membranes.

As a successful persistent virus, HIV-1 has evolved Env spikes that minimize the elicitation and impact of neutralizing antibodies. These features include surface variability, conformational lability and a heavy coat of glycans. Most anti-Env antibodies elicited during natural infection do not neutralize HIV-1, and those that do are usually strain-restricted, allowing virus escape. Only after several years of infection in some HIV-1-infected individuals are more broadly neutralizing antibodies generated. Broadly HIV-1-neutralizing antibodies typically display unusual features that allow binding to the heavily shielded, conserved Env epitopes. Some neutralizing antibodies with modest breadth bind Env carbohydrate-dependent epitopes. The variable and glycosylated features of the HIV-1 Env spike render the elicitation of neutralizing antibodies difficult, and have presented extreme challenges to the development of effective Env vaccine immunogens. Even the best current HIV-1 Env immunogens elicit antibodies that inhibit the infection only of the small subset of primary viruses that are more prone to neutralization. The sensitivity of HIV-1 strains to antibody neutralization depends upon the integrity of the Env epitope and Env reactivity the latter property indicates the propensity of unliganded Env to undergo conformational changes. A successful HIV-1 vaccine must cover a range of phylogenetically diverse transmitted/founder viruses, most of which have Envs of low reactivity and thus exhibit low sensitivity to neutralization by antibodies.

One of the major hurdles facing the development of a successful HIV-1/AIDS vaccine is the requirement to elicit antibodies that recognize conserved elements of the native, unliganded conformation of the HIV-1 Env trimer. These conserved elements are often buried or composed partially or in some cases completely of glycans, which render the generation of the cognate antibodies inefficient. Two functionally conserved gp120 elements interact with the HIV-1 host cell receptors. CD4 and CCR5/CXCR4. The CD4-binding site (CD4BS) on gp120 is sterically recessed on the HIV-1 Env trimer and surrounded by regions that exhibit inter-strain variability and glycosylation. Effective neutralizing antibodies directed against the gp120 CD4BS typically engage their epitopes in a manner that does not require the Env trimer to undergo significant conformational changes. Indeed, potently neutralizing antibodies directed against multiple conserved HIV-1 Env epitopes generally require minimal conformational change in the unliganded Env trimer for their binding.

The vast majority of primary HIV-1 isolates, including transmitted/founder viruses, use CCR5 as a second receptor. The CCR5-binding site on gp120 consists of a discontinuous surface of the gp120 core and the tip of the V3 loop, both of which are well conserved among primate immunodeficiency viruses. These elements are not formed and exposed on HIV-1 Env trimers with low envelope reactivity. Antibodies that recognize CD4-induced (CD4i) epitopes in the gp120 core bind near or within the coreceptor-binding site of gp120. Some of these antibodies are specific for CCR5-using HIV-1 variants, whereas other antibodies recognize both CCR5-using and CXCR4-using viruses. CD4i antibodies are routinely generated in HIV-1-infected humans, and can be elicited by HIV-1 gp120 core constructs in which the CD4-bound conformation has been stabilized by disulfide bonds and cavity-filling substitutions. Although both the CD4i epitopes and the V3 tip become exposed after HIV-1 binding to cell-surface CD4, steric factors (e.g., the target cell membrane) limit the ability of CD4i and V3-directed antibodies to bind their respective epitopes and neutralize the virus. Therefore, the neutralizing potency of CD4i and V3-directed antibodies is related to the degree of exposure of these epitopes on the unliganded Env trimer. Thus, because of the low Env reactivity of primary and transmitted/founder HIV-1, these viruses are generally inhibited poorly by most CD4i and V3-directed antibodies.

There exists a need for methods of eliciting antibodies that bind the unliganded HIV-1 Env trimer efficiently and neutralize the large fraction of primary transmitted/founder HIV-1 with low Env reactivity.

Furthermore, induction of the CD4-bound conformation renders primary HIV-1 sensitive to neutralization by CD4i antibodies. HIV-1 sensitization as a strategy for virus prophylaxis has become feasible as a result of the availability of small-molecule CD4-mimetic compounds. The prototypes of such compounds, NBD-556 and NBD-557, were discovered in a screen for inhibitors of gp120-CD4 interaction. NBD-556 and NBD-557 bind in the Phe 43 cavity, a highly conserved ˜150 cubic Angstrom pocket in the gp120 glycoprotein of all HIV-1 strains except those in Group O. The vestibule of the Phe 43 cavity contains a number of conserved gp120 residues that make critical contacts with CD4. The binding of NBD compounds in the Phe 43 cavity blocks gp120-CD4 interaction and, like the binding of soluble CD4, prematurely triggers the activation of the HIV-1 Env spike. The activated state is short-lived (t_{1/2}=5-7 minutes at 37° C.) and the bound Env spike rapidly decays into an irreversibly inactivated state. Although NBD-556 induces large, entropically unfavorable changes in gp120 conformation and thus binds with only modest affinity (K_{d}=3 μM), iterative cycles of co-crystallization with gp120 and rational design and synthesis have yielded a number of NBD-556 analogues with improved affinity and antiviral properties. However, NBD-556 suffers from one significant disadvantage with respect to development of a vaccine: it increases the binding or neutralizing potency of the 17b CD4i antibody weakly and only in laboratory-adapted viruses that have high Env reactivity.

There also exists a need for methods of increasing the sensitivity of the HIV-1 virion to antibody neutralization.

In addition, there is increasing evidence supporting a role of Antibody-Dependent Cell-Mediated Cytotoxicity (ADCC) in controlling HIV-1 transmission and disease progression. A recent vaccine trial suggested that increased ADCC activity was linked with decreased HIV-1 acquisition; indeed, antibodies with potent ADCC activity were isolated from some of these vaccines. It has also been reported that HIV-1 Env in the CD4-bound conformation is preferentially targeted by ADCC-mediating antibodies. However, viral accessory proteins Nef and Vpu protect HIV-1 infected cells from Env-mediated ADCC responses. Unfortunately, the vast majority of circulating HIV-1 strains worldwide express functional Nef and Vpu proteins, so the exposure of CD4-induced (CD4i) Env epitopes at the surface of infected cells is limited and ADCC responses are impeded.

There also exists a need for methods of sensitizing HIV-1 infected cells to ADCC.

### SUMMARY OF THE DISCLOSURE

In certain embodiments, the disclosure relates to methods of generating a protein binding domain that specifically binds to gp120 in a specific conformational state, the method comprising the steps of:

a) contacting gp120 or a fragment thereof with a compound, wherein the compound is a compound of Formula VII, Formula I, Formula II, Formula III, Formula IV, Formula V, Formula VI, or Formula VIII, thereby forming gp120 or the fragment thereof in the specific conformational state; and

b) generating antibodies to gp120 or the fragment thereof in the specific conformation state, wherein the compound is optionally bound to the gp120 or the fragment thereof in the specific conformational state.

In certain embodiments, the disclosure relates to methods of neutralizing HIV-1, the method comprising the step of:
contacting HIV-1 with an effective amount of a compound of Formula VII, Formula I, Formula II, Formula III, Formula IV, Formula V, Formula VI, or Formula VIII, thereby forming HIV-1 having gp120 in a specific conformational state; and
contacting the HIV-1 in the specific conformational state with an antibody, wherein the compound is optionally bound to the HIV-1 in the specific conformational state.

In certain embodiments, the disclosure relates to methods of treating or preventing HIV infection, the method comprising the step of:
administering to a subject in need thereof, a therapeutically effective amount of an antibody; and
co-administering to the subject an effective amount of a compound of Formula VII, Formula I, Formula II, Formula III, Formula IV, Formula V, Formula VI, or Formula VIII.

In certain embodiments, the disclosure relates to a complex comprising (i) a compound of Formula VII, Formula I, Formula II, Formula III, Formula IV, Formula V, Formula VI, or Formula VIII, (ii) gp120 in a functional conformational state, and (iii) optionally, an antibody.

### BRIEF DESCRIPTION OF THE FIGURES

FIG. 1 has four panels (A, B, C, and D) depicting the effects of NBD-556 and analogues on HIV-1 infection. The infection of Cf2Th-CD4/CCR5 by recombinant HIV-1 expressing firefly luciferase was measured in the presence of the indicated concentrations of NBD-556 (panel A), (+)-MAE-II-120 (panel B). (+)-DMJ-I-228 (panel C), and (+)-DMJ-II-121 (panel D). The viruses contained the HIV-1_{JR-FL} or amphotropic murine leukemia (A-MLV) envelope glycoproteins. The values are represented as a percentage of the level of target cell luciferase observed for each virus in the absence of the compound. The means and standard deviations of the results obtained in triplicate samples are shown. The results are representative of those obtained in 3-21 independent experiments.

FIG. 2 has six panels (A, B, C, D, E, and F) depicting the results associated with the sensitization of HIV-1_{JR-FL} by (+)-DMJ-II-121 to neutralization by the 17b and 39F antibodies. (Panel A, panel B): The neutralization of HIV-1_{JR-FL} by the CD4-induced antibody 17b (panel A) and the V3-directed antibody 39F (panel B) is shown in the presence of the indicated concentrations of (+)-DMJ-II-121. (Panel C, panel D): Neutralization of virus with the A-MLV envelope glycoprotein by the 17b (panel C) or 39F (panel D) antibodies is shown in the presence or the absence of 50 μM (+)-DMJ-II-121. (Panel E, panel F): Infection by viruses with HIV-1_{JR-FL} or A-MLV envelope glycoproteins in the presence of the indicated concentrations of 17b (panel E) or 39F (panel F) antibody and 100 μM NBD-556. All viral infection data shown is normalized to the level of infection seen in the absence of antibody at the indicated concentration of (+)-DMJ-II-121. The means and standard deviations of the results obtained in triplicate samples are shown. The results are representative of those obtained in 4-10 independent experiments.

FIG. 3 has five panels (A, B, C, D, and E) depicting (+)-DMJ-II-121 sensitization of HIV-1 to neutralization by a CD4-induced antibody from an HIV-1-infected individual and a rabbit antiserum elicited by the 3CC stabilized HIV-1 gp120 core. A concentration of (+)-DMJ-II-121 that reduced HIV-1_{JR-FL} and 3A1 transmitted/founder virus infection by approximately 50% was used in these experiments. The effects of increasing concentrations of rabbit antiserum elicited by the 3CC stabilized gp120 core on infection by HIV-1_{JR-FL} (panel A) or the A-MLV-pseudotyped HIV-1 control (panel B) are shown. The infection of the B5 transmitted/founder virus in the presence of the indicated concentrations of the 17b antibody (panel C) are shown, in the absence and presence of 50 μM (+)-DMJ-II-121. The effects of increasing concentrations of the CD4-induced antibody (CH08), which was derived from an HIV-1-infected individual (88), on infection by HIV-1_{JR-FL} (panel D) and the A-MLV-pseudotyped HIV-1 control (panel E) are shown, in the absence and presence of 50 μM (+)-DMJ-II-121. All viral infection data shown are normalized to the level of infection seen in the absence of antibody at the indicated concentration of (+)-DMJ-II-121. The means and standard deviations of the results obtained in triplicate samples are shown. The results are representative of those obtained in 2-20 independent experiments.

FIG. 4 tabulates properties of exemplary small-molecule CD-4 mimetic compounds. ^{a}R/S designation refers to the absolute stereochemical configuration at the 1 and 2 positions of the indane ring system in the compound. ^{b}The thermodynamic data were determined by isothermal titration calorimetry at 25° C. ^{c}The half-life of HIV-1 JR-FL infectivity on ice in the presence of the CD4-mimetic compound (50 μM) is shown. The half-life of HIV-1 JR-FL on ice in DMSO was >48 h. ^{d}The IC₅₀ of the 17b antibody in μg/mL is shown for neutralization of HIV-1 JR-FL in the presence of 50 μM of the compound.

FIG. 5 tabulates the inhibitory concentrations (IC₅₀) (μg/mL) of the 17b and 39F antibodies for the HIV-1-JR-FL, HIV-1 YU2 and A-MLV viruses. ^{a}The IC₅₀ of the 17b and 39F antibodies for the indicated viruses in the absence or presence of the indicated concentrations of (+)-DMJ-I-228 or (+)-DMJ-II-121 is reported. In pilot experiments. (4)-DMJ-I-228 inhibited HIV-1 JR-FL and HIV-1 YU2 infections with IC₅₀'s of 86.9 and 46.7 μM, respectively. (+)-DMJ-I-121 inhibited HIV-1 JR-FL and HIV-1 YU2 with ICs's of 27.8 and 3.6 μM, respectively. Neither (+)-DMJ-I-228 nor (+)-DMJ-II-121 inhibited A-MLV infection at 100 μM. The number of experiments is shown in parentheses. ND=Not determined.

FIG. 6 tabulates the inhibitory concentrations (IC₅₀) (μg/mL) of antibodies for infection by HIV-1-JR-FL and A-MLV in the absence or presence of 50 μm (+)-DMJ-II-121. In some experiments, 80 μM (+)-DMJ-II-121 was used, as indicated in parentheses.

FIG. 7 tabulates neutralization titers of sera from rabbits immunized with stabilized and unmodified gp120 cores, tested against HIV-1 JR-FL in the absence or presence of (+)-DMJ-II-121. ^{a}The numbers in parentheses indicate the numbers of rabbits with the reported scrum titer/total number of rabbits in the group.

FIG. 8A tabulates inhibitory concentrations (IC₅₀) of (+)-DMJ-II-121 and the 17b antibody (alone and in the presence of (+)-DMJ-II-121), tested against HIV-1 JR-FL, transmitted/founder virus, and A-MLV. The IC₅₀ of (+)-DMJ-II-121 and the 17b antibody (alone and in the presence of 50 μM (+)-DMJ-II-121) is reported. The number of experiments is shown in parentheses; unless indicated, the reported IC₅₀ value is derived from 3-5 independent experiments.

FIG. 8B tabulates inhibitory concentrations (IC₅₀ in dilutions of sera) of pre-immune sera and sera from rabbits immunized with the 3CC stabilized gp120 core, tested against HIV-1 JR-FL, transmitted/founder virus, and A-MLV in the presence or absence of (+)-DMJ-II-121.

FIG. 9 depicts a schematic representation of four different levels of theoretical protection imparted by CD4-mimetic compounds in combination with antibodies during sexual transmission of the virus.

### DETAILED DESCRIPTION OF THE DISCLOSURE

### Overview

Approaches to prevent human immunodeficiency virus (HIV-1) transmission are urgently needed. Difficulties in eliciting antibodies that bind conserved epitopes exposed on the unliganded conformation of the HIV-1 envelope glycoprotein (Env) trimer represent barriers to vaccine development. During HIV-1 entry, binding of the gp120 Env to the initial receptor, CD4, triggers conformational changes in Env that result in the formation and exposure of the highly conserved gp120 site for interaction with the coreceptors, CCR5 or CXCR4. Compounds such as (+)-DMJ-1-228 and (+)-DMJ-II-121

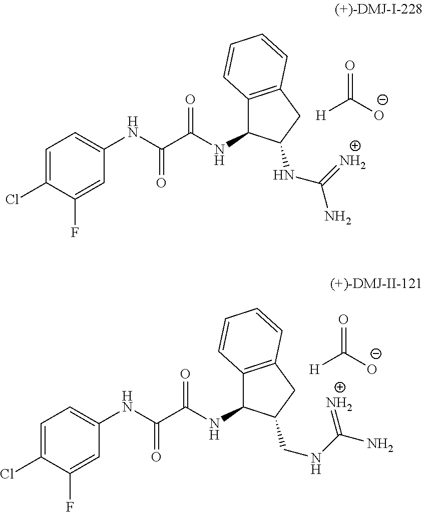

bind gp120 within the conserved Phe 43 cavity near the CD4-binding site, block CD4 binding and inhibit HIV-1 infection with IC₅₀ values of 22.9 and 2.3 μM, respectively.

In certain embodiments, the disclosure relates to methods of sensitizing primary HIV-1, including transmitted/founder viruses, to neutralization by monoclonal antibodies directed against CD4-induced (CD4i) epitopes and the V3 region, two gp120 elements involved in coreceptor binding. In certain embodiments, the disclosure relates to the sensitization of primary HIV-1 by small-molecule compounds to neutralization by antisera elicited by immunization of rabbits with HIV-1 gp120 cores engineered to assume the CD4-bound state. In certain embodiments, the disclosure relates to the use of small molecules like (+)-DMJ-I-228 and (+)-DMJ-II-121 as microbicides to inhibit HIV-1 infection directly and to sensitize primary HIV-1 to neutralization by readily elicited antibodies. In certain embodiments, the virus-sensitizing activity of the small-molecule compounds is robust. In certain embodiments, the virus-sensitizing activity of the small-molecule compounds is evident in primary HIV-1 isolates that have low Env reactivity and thus are relatively neutralization-resistant.

An attractive strategy for preventing HIV-1 acquisition is to generate antibodies in an uninfected individual that potently neutralize a wide range of transmitted/founder HIV-1. Both viral and antibody factors determine HIV-1 neutralization efficiency. Transmitted/founder viruses generally exhibit low Env reactivity and thus are relatively resistant to neutralization. Antibodies that effectively neutralize these low-reactivity viruses must bind the unliganded Env trimer efficiently, without requiring significant conformational changes in Env. The many ongoing efforts to elicit such neutralizing antibodies have yet to succeed. In certain embodiments, the disclosure relates to an approach that increases the sensitivity of the HIV-L virion to some neutralizing antibodies. In certain embodiments, the approach takes advantage of: 1) the natural tendency of HIV-1 Env to make the transition from the unliganded state to the CD4-bound state; 2) the highly conserved nature of the gp120 binding sites for CD4 and CCR5; 3) the vulnerability to antibody neutralization of the CD4-bound state of Env on a virus that is distant from the target membrane; and 4) the availability of small-molecule CD4-mimetic compounds that exhibit sufficient affinity and breadth.

Compared with the parental NBD-556 compound, (+)-DMJ-I-228 and (+)-DMJ-II-121 bind gp120 with higher affinity, block gp120-CD4 binding more efficiently, and inhibit HIV-1 infection with lower IC₅₀ values. NBD-556 has been shown to increase the binding of the 17b CD4i antibody to gp120, and has been reported to increase weakly the ability of the 17b CD4i antibody to neutralize laboratory-adapted viruses, which have high envelope reactivity. In certain embodiments, (+)-DMJ-I-228 and (+)-DMJ-II-121 each sensitize primary HIV-1 isolates, which have low envelope reactivity and are relatively neutralization resistant, to inhibition by specific anti-gp120 antibodies. In certain embodiments, the disclosure relates to the discovery that, in the presence of (+)-DMJ-II-121, multiple primary HIV-1, including transmitted/founder HIV-1, were sensitive to neutralization by the 17b antibody or antisera elicited by a 3CC gp120 core immunogen. Importantly, in certain embodiments, the observed sensitization seen with the viruses was dependent on the binding of (+)-DMJ-I-228 or (+)-DMJ-II-121 to the viral Env. In certain embodiments, (+)-DMJ-I-228 and (+)-DMJ-II-121, like their NBD predecessors, do not bind to the S375W variant of HIV-1 Env, where the Phe 43 cavity is filled and therefore unavailable for compound binding. In certain embodiments, sensitization of HIV-1 to neutralization by antibodies apparently requires sufficient affinity of the CD4-mimetic compound for Env. In contrast to NBD-556, (+)-DMJ-I-228 and (+)-DMJ-II-121 each contact the conserved gp120 residue, Asp 368, which makes important contributions to CD4 binding. (+)-DMJ-II-121 also makes an additional interaction with Met 426. Recent results from alanine scanning have shown that interactions with different residues in gp120 contribute differently to binding affinity and conformational structuring; Asp 368 and, in particular, Met 426 contribute significantly to binding and to a lesser extent to conformational structuring. In certain embodiments, while not wishing to be bound by any particular theory, the interaction of (+)-DMJ-I-228 or (+)-DMJ-II-121 with these residues may explain why (+)-DMJ-I-228 and (+)-DMJ-II-121 bind with a smaller entropic penalty than NBD-556. In certain embodiments, while not wishing to be bound by any particular theory, the interaction of (+)-DMJ-I-228 or (+)-DMJ-II-121 with gp120 points toward a binding event that is characterized by a better affinity and a coupling to some different conformational changes in Env that are smaller in magnitude and necessary for the sensitization effect.

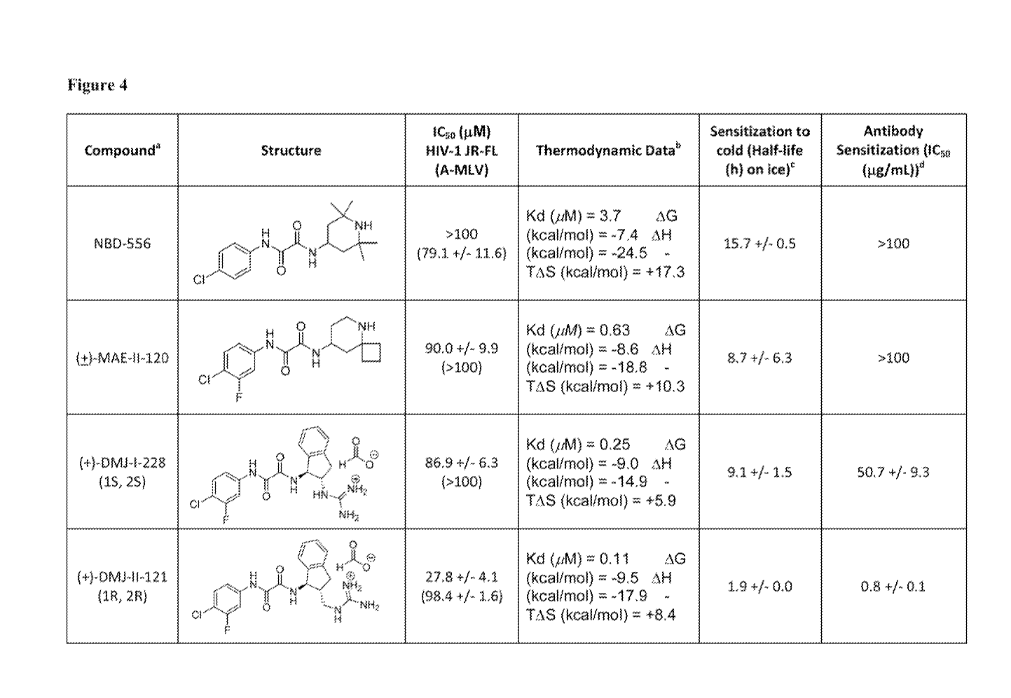

In certain embodiments, the CD4i and V3-directed anti-gp120 antibodies neutralized HIV-1 with dramatically improved potency in the presence of (+)-DMJ-I-228 or (+)-DMJ-II-121. These two groups of antibodies recognize gp120 epitopes that share several features: 1) poor formation/exposure on the unliganded HIV-1 Env trimer; 2) induction by CD4 binding; 3) involvement in coreceptor binding; and 4) a high degree of conservation in the components of the epitope that interact with the coreceptor. In certain embodiments, while not wishing to be bound by any particular theory, as the sensitizing effect of (+)-DMJ-I-228 and (−)-DMJ-II-121 did not extend to other groups of Env-directed antibodies, sensitization of HIV-1 likely results from the induction of Env conformational changes similar to those induced by CD4. In certain embodiments, while not wishing to be bound by any particular theory, this conclusion is consistent with the documented CD4-mimetic thermodynamic and entry-enhancing properties of NBD-556 analogues, as well as the proximity of their gp120 binding site to that of CD4. In addition, the sensitization of HIV-1 to CD4i and V3-directed antibodies by different NBD-556 analogues correlated with sensitization of the virus to cold inactivation (FIG. 4). The sensitivity of HIV-1 variants to cold inactivation is known to increase in proportion to their tendency to assume the CD4-bound conformation. Thus, the observed relationship between sensitization of HIV-1 to antibody neutralization and cold inactivation is consistent with different levels of induction of the CD4-bound state by the various NBD-556 analogues. In summary, the sensitization of HIV-1 by (+)-DMJ-1-228 or (+)-DMJ-II-121 apparently involves the induction of conformational changes related to those that occur during CD4 binding.

In certain embodiments, the disclosure relates to methods of enhancing vaccine efficacy comprising the step of co-administering (+)-DMJ-I-228 or (+)-DMJ-II-121. One frustrating aspect of HIV-1 vaccine development is the difficulty of eliciting antibodies that potently neutralize diverse strains of virus. Sensitization of HIV-1, including transmitted/founder viruses, by (+)-DMJ-I-228 or (+)-DMJ-II-121 results in a virus that is neutralizable by antibodies that can be readily elicited. During HIV-1 infection of humans, CD4i antibodies are elicited early and in a high proportion of infected individuals; this suggests that the generation of such antibodies in humans may be achievable by vaccination. Moreover, “stabilized gp120 cores” that have been engineered to assume the CD4-bound state have been demonstrated to raise CD4i antibodies in immunized rabbits. In certain embodiments, the disclosure relates to the discovery that (+)-DMJ-II-121 sensitizes primary HIV-1 JR-FL and transmitted/founder viruses to polyclonal sera raised by the 3CC and 4CC stabilized cores in multiple rabbits. Thus, the two fundamental components of a prophylactic approach based on HIV-1 sensitization are in place: 1) (+)-DMJ-I-228 and (+)-DMJ-II-121 that inhibit HIV-L entry and also sensitize HIV-1 to neutralization by CD4i and V3-directed antibodies; and 2) stabilized gp120 core immunogens that can elicit CD4i antibodies. In certain embodiments, the disclosure relates to a multi-component vaccine regimen in which one of the immunogens is a stabilized gp120 core that elicits antibodies against the conserved coreceptor-binding site. (+)-DMJ-I-228 and (+)-DMJ-I-121 administered orally or in a microbicide formulation could sensitize a range of transmitted/founder viruses to inhibition by the vaccine-elicited antibodies.

### DEFINITIONS

In order for the present disclosure to be more readily understood, certain terms and phrases are defined below and throughout the specification.

The articles “a” and “an” are used herein to refer to one or to more than one (i.e., to at least one) of the grammatical object of the article. By way of example, “an element” means one element or more than one element.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of,” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one. B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including.” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

The definition of each expression, e.g., alkyl, m, n, and the like, when it occurs more than once in any structure, is intended to be independent of its definition elsewhere in the same structure.

It will be understood that “substitution” or “substituted with” includes the implicit proviso that such substitution is in accordance with permitted valence of the substituted atom and the substituent, and that the substitution results in a stable compound. e.g., a compound which does not spontaneously undergo transformation such as by rearrangement, cyclization, elimination, or other reaction.

The term “substituted” is also contemplated to include all permissible substituents of organic compounds. In a broad aspect, the permissible substituents include acyclic and cyclic, branched and unbranched, carbocyclic and heterocyclic, aromatic and nonaromatic substituents of organic compounds. Illustrative substituents include, for example, those described herein below. The permissible substituents may be one or more and the same or different for appropriate organic compounds. For purposes of this disclosure, the heteroatoms such as nitrogen may have hydrogen substituents and/or any permissible substituents of organic compounds described herein which satisfy the valences of the heteroatoms. This disclosure is not intended to be limited in any manner by the permissible substituents of organic compounds.

The term “lower” when appended to any of the groups listed below indicates that the group contains less than seven carbons (i.e. six carbons or less). For example “lower alkyl” refers to an alkyl group containing 1-6 carbons, and “lower alkenyl” refers to an alkenyl group containing 2-6 carbons.

The term “saturated,” as used herein, pertains to compounds and/or groups which do not have any carbon-carbon double bonds or carbon-carbon triple bonds.

The term “unsaturated,” as used herein, pertains to compounds and/or groups which have at least one carbon-carbon double bond or carbon-carbon triple bond.

The term “aliphatic,” as used herein, pertains to compounds and/or groups which are linear or branched, but not cyclic (also known as “acyclic” or “open-chain” groups).

The term “cyclic,” as used herein, pertains to compounds and/or groups which have one ring, or two or more rings (e.g., spiro, fused, bridged).

The term “aromatic” refers to a planar or polycyclic structure characterized by a cyclically conjugated molecular moiety containing 4n+2 electrons, wherein n is the absolute value of an integer. Aromatic molecules containing fused, or joined, rings also are referred to as bicyclic aromatic rings. For example, bicyclic aromatic rings containing heteroatoms in a hydrocarbon ring structure are referred to as bicyclic heteroaryl rings.

The term “hydrocarbon” as used herein refers to an organic compound consisting entirely of hydrogen and carbon.

For purposes of this disclosure, the chemical elements are identified in accordance with the Periodic Table of the Elements, CAS version, Handbook of Chemistry and Physics, 67th Ed., 1986-87, inside cover.

The term “heteroatom” as used herein is art-recognized and refers to an atom of any element other than carbon or hydrogen. Illustrative heteroatoms include boron, nitrogen, oxygen, phosphorus, sulfur and selenium.

The term “alkyl” means an aliphatic or cyclic hydrocarbon radical containing from 1 to 12 carbon atoms. Representative examples of alkyl include, but are not limited to, methyl, ethyl, n-propyl, iso-propyl, n-butyl, sec-butyl, iso-butyl, tert-butyl, n-pentyl, isopentyl, neopentyl, n-hexyl, 2-methylcyclopentyl, and 1-cyclohexylethyl.

The term “substituted alkyl” means an aliphatic or cyclic hydrocarbon radical containing from 1 to 12 carbon atoms, substituted with 1, 2, 3, 4, or 5 substituents independently selected from the group consisting of alkyl, alkenyl, alkynyl, halo, haloalkyl, fluoroalkyl, hydroxy, alkoxy, alkenyloxy, alkynyloxy, carbocyclyloxy, heterocyclyloxy, haloalkoxy, fluoroalkyloxy, sulfhydryl, alkylthio, haloalkylthio, fluoroalkylthio, alkenylthio, alkynylthio, sulfonic acid, alkylsulfonyl, haloalkylsulfonyl, fluoroalkylsulfonyl, alkenylsulfonyl, alkynylsulfonyl, alkoxysulfonyl, haloalkoxysulfonyl, fluoroalkoxysulfonyl, alkenyloxysulfonyl, alkynyloxysulfonyl, aminosulfonyl, sulfinic acid, alkylsulfinyl, haloalkylsulfinyl, fluoroalkylsulfinyl, alkenylsulfinyl, alkynylsulfinyl, alkoxysulfinyl, haloalkoxysulfinyl, fluoroalkoxysulfinyl, alkenyloxysulfinyl, alkynyloxysulfinyl, aminosulfinyl, formyl, alkylcarbonyl, haloalkylcarbonyl, fluoroalkylcarbonyl, alkenylcarbonyl, alkynylcarbonyl, carboxy, alkoxycarbonyl, haloalkoxycarbonyl, fluoroalkoxycarbonyl, alkenyloxycarbonyl, alkynyloxycarbonyl, alkylcarbonyloxy, haloalkylcarbonyloxy, fluoroalkylcarbonyloxy, alkenylcarbonyloxy, alkynylcarbonyloxy, alkylsulfonyloxy, haloalkylsulfonyloxy, fluoroalkylsulfonyloxy, alkcnylsulfonyloxy, alkynylsulfonyloxy, haloalkoxysulfonyloxy, fluoroalkoxysulfonyloxy, alkenyloxysulfonyloxy, alkynyloxysulfonyloxy, alkylsulfinyloxy, haloalkylsulfinyloxy, fluoroalkylsulfinyloxy, alkenylsulfinyloxy, alkynylsulfinyloxy, alkoxysulfinyloxy, haloalkoxysulfinyloxy, fluoroalkoxysulfinyloxy, alkenyloxysulfinyloxy, alkynyloxysulfinyloxy, aminosulfinyloxy, amino, amido, aminosulfonyl, aminosulfinyl, cyano, nitro, azido, phosphinyl, phosphonyl, silyl and silyloxy.

The term “carbocyclyl” as used herein means monocyclic or multicyclic (e.g., bicyclic, tricyclic, etc.) hydrocarbons containing from 3 to 12 carbon atoms that is completely saturated or has one or more unsaturated bonds, and for the avoidance of doubt, the degree of unsaturation does not result in an aromatic ring system (e.g. phenyl). Examples of carbocyclyl groups include 1-cyclopropyl, 1-cyclobutyl, 2-cyclopentyl, 1-cyclopcetenyl, 3-cyclohexyl, 1-cyclohexenyl and 2-cyclopentenylmethyl.

The term “heterocyclyl”, as used herein include non-aromatic, ring systems, including, but not limited to, monocyclic, bicyclic (e.g. fused and spirocyclic) and tricyclic rings, which can be completely saturated or which can contain one or more units of unsaturation, for the avoidance of doubt, the degree of unsaturation does not result in an aromatic ring system, and have 3 to 12 atoms including at least one heteroatom, such as nitrogen, oxygen, or sulfur. For purposes of exemplification, which should not be construed as limiting the scope of this disclosure, the following are examples of heterocyclic rings: azepines, azetidinyl, morpholinyl, oxopiperidinyl, oxopyrrolidinyl, piperazinyl, piperidinyl, pyrrolidinyl, quinicludinyl, thiomorpholinyl, tetrahydropyranyl and tetrahydrofuranyl. The heterocyclyl groups of the disclosure are substituted with 0, 1, 2, 3, 4 or 5 substituents independently selected from the group consisting of alkyl, alkenyl, alkynyl, halo, haloalkyl, fluoroalkyl, hydroxy, alkoxy, alkenyloxy, alkynyloxy, carbocyclyloxy, heterocyclyloxy, haloalkoxy, fluoroalkyloxy, sulfhydryl, alkylthio, haloalkylthio, fluoroalkylthio, alkenylthio, alkynylthio, sulfonic acid, alkylsulfonyl, haloalkylsulfonyl, fluoroalkylsulfonyl, alkenylsulfonyl, alkynylsulfonyl, alkoxysulfonyl, haloalkoxysulfonyl, fluoroalkoxysulfonyl, alkenyloxysulfonyl, alkynyloxysulfonyl, aminosulfonyl, sulfinic acid, alkylsulfinyl, haloalkylsulfinyl, fluoroalkylsulfinyl, alkenylsulfinyl, alkynylsulfinyl, alkoxysulfinyl, haloalkoxysulfinyl, fluoroalkoxysulfinyl, alkenyloxysulfinyl, alkynyloxysulfinyl, aminosulfinyl, formyl, alkylcarbonyl, haloalkylcarbonyl, fluoroalkylcarbonyl, alkenylcarbonyl, alkynylcarbonyl, carboxy, alkoxycarbonyl, haloalkoxycarbonyl, fluoroalkoxycarbonyl, alkenyloxycarbonyl, alkynyloxycarbonyl, alkylcarbonyloxy, haloalkylcarbonyloxy, fluoroalkylcarbonyloxy, alkenylcarbonyloxy, alkynylcarbonyloxy, alkylsulfonyloxy, haloalkylsulfonyloxy, fluoroalkylsultbnyloxy, alkenylsulfonyloxy, alkynylsulfonyloxy, haloalkoxysulfonyloxy, fluoroalkoxysulfonyloxy, alkenyloxysulfonyloxy, alkynyloxysnlfonyloxy, alkylsulfinyloxy, haloalkylsulfinyloxy, fluoroalkylsulfinyloxy, alkenylsulfinyloxy, alkynylsulfinyloxy, alkoxysulfinyloxy, haloalkoxysulfinyloxy, fluoroalkoxysulfinyloxy, alkenyloxysulfinyloxy, alkynyloxysulfinyloxy, aminosufinyloxy, amino, amido, aminosulfonyl, aminosulfinyl, cyano, nitro, azido, phosphinyl, phosphoryl, silyl, silyloxy, and any of said substituents bound to the heterocyclyl group through an alkylene moiety (e.g. methylene).

The term “N-heterocyclyl” as used herein is a subset of heterocyclyl, as defined herein, which have at least one nitrogen atom through which the N-heterocyclyl moiety is bound to the parent moiety. Representative examples include pyrrolidin-1-yl, piperidin-1-yl, piperazin-1-yl, hexahydropyrimidin-1-yl, morpholin-1-yl, 1,3-oxazinan-3-yl and 6-azaspiro[2.5]oct-6-yl. As with the heterocyclyl groups, the N-heterocyclyl groups of the disclosure are substituted with 0, 1, 2, 3, 4 or 5 substituents independently selected from the group consisting of alkyl, alkenyl, alkynyl, halo, haloalkyl, fluoroalkyl, hydroxy, alkoxy, alkenyloxy, alkynyloxy, carbocyclyloxy, heterocyclyloxy, haloalkoxy, fluoroalkyloxy, sulfhydryl, alkylthio, haloalkylthio, fluoroalkylthio, alkenylthio, alkenylthio, sulfonic acid, alkylsulfonyl, haloalkylsulfonyl, fluoroalkylsulfonyl, alkenylsulfonyl, alkynylsulfonyl, alkoxysulfonyl, haloalkoxysulfonyl, fluoroalkoxysulfonyl, alkcnyloxysulfonyl, alkynyloxysulfonyl, amninosulfonyl, sulfinic acid, alkylsulfinyl, haloalkylsulfinyl, fluoroalkylsulfinyl, alkenylsulfinyl, alkynylsulfinyl, alkoxysulfinyl, haloalkoxysultinyl, fluoroalkoxysulfinyl, alkenyloxysulfinyl, alkynyloxysulfinyl, aminosulfinyl, formyl, alkylcarbonyl, haloalkylcarbonyl, fluoroalkylcarbonyl, alkcnylcarbonyl, alkynylcarbonyl, carboxy, alkoxycarbonyl, haloalkoxycarbonyl, fluoroalkoxycarbonyl, alkenyloxycarbonyl, alkynyloxycarbonyl, alkylcarbonyloxy, haloalkylcarbonyloxy, fluoroalkylcarbonyloxy, alkenylcarbonyloxy, alkynylcarbonyloxy, alkylsulfonyloxy, haloalkylsulfonyloxy, fluoroalkylsulfonyloxy, alkenylsulfonyloxy, alkynylsulfonyloxy, haloalkoxysulfonyloxy, fluoroalkoxysulfonyloxy, alkenyloxysulfonyloxy, alkynyloxysulfonyloxy, alkylsulfinyloxy, haloalkylsulfinyloxy, fluoroalkylsulfinyloxy, alkenylsulfinyloxy, alkynylsulfinyloxy, alkoxysulfinyloxy, haloalkoxysulfinyloxy, fluoroalkoxysulfinyloxy, alkcnyloxysulfinyloxy, alkynyloxysulfinyloxy, aminosulfinyloxy, amino, amido, aminosulfonyl, aminosulfinyl, cyano, nitro, azido, phosphinyl, phosphoryl, silyl, silyloxy, and any of said substituents bound to the N-heterocyclyl group through an alkylene moiety (e.g. methylene).

The term “aryl,” as used herein means a phenyl group, naphthyl or anthracenyl group. The aryl groups of the present disclosure can be optionally substituted with 1, 2, 3, 4 or 5 substituents independently selected from the group consisting of alkyl, alkenyl, alkynyl, halo, haloalkyl, fluoroalkyl, hydroxy, alkoxy, alkenyloxy, alkynyloxy, carbocyclyloxy, heterocyclyloxy, haloalkoxy, fluoroalkyloxy, sulfhydryl, alkylthio, haloalkylthio, fluoroalkylthio, alkenylthio, alkynylthio, sulfonic acid, alkylsulfonyl, haloalkylsulfonyl, fluoroalkylsulfonyl, alkenylsulfonyl, alkynylsulfonyl, alkoxysulfonyl, haloalkoxysulfbnyl, fluoroalkoxysulfonyl, alkenyloxysulfonyl, alkynyloxysulfonyl, aminosulfonyl, sulfinic acid, alkylsulfinyl, haloalkylsulfinyl, fluoroalkylsulfinyl, alkenylsulfinyl, alkynylsulfinyl, alkoxysulfinyl, haloalkoxysulfinyl, fluoroalkoxysulfinyl, alkenyloxysulfinyl, alkynyloxysulfinyl, aminosulfinyl, formyl, alkylcarbonyl, haloalkylcarbonyl, fluoroalkylcarbonyl, alkenylcarbonyl, alkynylcarbonyl, carboxy, alkoxycarbonyl, haloalkoxycarbonyl, fluoroalkoxycarbonyl, alkenyloxycarbonyl, alkynyloxycarbonyl, alkylcarbonyloxy, haloalkylcarbonyloxy, fluoroalkylcarbonyloxy, alkenylcarbonyloxy, alkynylcarbonyloxy, alkylsulfonyloxy, haloalkylsulfonyloxy, fluoroalkylsulfonyloxy, alkenylsulfonyloxy, alkynylsulfonyloxy, haloalkoxysulfonyloxy, fluoroalkoxysulfonyloxy, alkenyloxysulfonyloxy, alkynyloxysulfonyloxy, alkylsulfinyloxy, haloalkylsulfinyloxy, fluoroalkylsulfinyloxy, alkenylsulfinyloxy, alkynylsulfinyloxy, alkoxysulfinyloxy, haloalkoxysulfinyloxy, fluoroalkoxysulfinyloxy, alkcnyloxysulfinyloxy, alkynyloxysulfinyloxy, aminosulfinyloxy, amino, amido, aminosulfonyl, aminosulfinyl, cyano, nitro, azido, phosphinyl, phosphoryl, silyl, silyloxy, and any of said substituents bound to the heterocyclyl group through an alkylene moiety (e.g. methylene).

The term “arylene,” is art-recognized, and as used herein pertains to a bidentate moiety obtained by removing two hydrogen atoms of an aryl ring, as defined above.

The term “arylalkyl” or “aralkyl” as used herein means an aryl group, as defined herein, appended to the parent molecular moiety through an alkyl group, as defined herein. Representative examples of aralkyl include, but are not limited to, benzyl, 2-phenylethyl, 3-phenylpropyl, and 2-naphth-2-ylethyl.

The term “heteroaryl” as used herein include aromatic ring systems, including, but not limited to, monocyclic, bicyclic and tricyclic rings, and have 3 to 12 atoms including at least one heteroatom, such as nitrogen, oxygen, or sulfur. For purposes of exemplification, which should not be construed as limiting the scope of this disclosure: azaindolyl, benzo(b)thienyl, benzimidazolyl, benzofuranyl, benzoxazolyl, benzothiazolyl, benzothiadiazolyl, benzotriazolyl, benzoxadiazolyl, furanyl, imidazolyl, imidazopyridinyl, indolyl, indolinyl, indazolyl, isoindolinyl, isoxazolyl, isothiazolyl, isoquinolinyl, oxadiazolyl, oxazolyl, purinyl, pyranyl, pyrazinyl, pyrazolyl, pyridinyl, pyrimidinyl, pyrrolyl, pyrrolo[2,3-d]pyrimidinyl, pyrazolo[3,4-d]pyrimidinyl, quinolinyl, quinazolinyl, triazolyl, thiazolyl, thiophenyl, tetrahydroindolyl, tetrazolyl, thiadiazolyl, thienyl, thiomorpholinyl, triazolyl or tropanyl. The heteroaryl groups of the disclosure are substituted with 0, 1, 2, 3, 4 or 5 substituents independently selected from the group consisting of alkyl, alkenyl, alkynyl, halo, haloalkyl, fluoroalkyl, hydroxy, alkoxy, alkenyloxy, alkynyloxy, carbocyclyloxy, heterocyclyloxy, haloalkoxy, fluoroalkyloxy, sulfhydryl, alkylthio, haloalkylthio, fluoroalkylthio, alkenylthio, alkynylthio, sulfonic acid, alkyLsulfonyl, haloalkylsulfonyl, fluoroalkylsulfonyl, alkenylsulfonyl, alkynylsulfonyl, alkoxysulfonyl, haloalkoxysulfonyl, fluoroalkoxysulfonyl, alkenyloxysulfonyl, alkynyloxysulfonyl, aminosulfonyl, sulfinic acid, alkylsulfinyl, haloalkylsulfinyl, fluoroalkylsulfinyl, alkenylsulfinyl, alkynylsulfinyl, alkoxysulfinyl, haloalkoxysulfinyl, fluoroalkoxysulfinyl, alkenyloxysulfinyl, alkynyloxysulfiny, aminosulfinyl, formyl, alkylcarbonyl, haloalkylcarbonyl, fluoroalkylcarbonyl, alkenylcarbonyl, alkynylcarbonyl, carboxy, alkoxycarbonyl, haloalkoxycarbonyl, fluoroalkoxycarbonyl, alkenyloxycarbonyl, alkynyloxycarbonyl, alkylcarbonyloxy, haloalkylcarbonyloxy, fluoroalkylcarbonyloxy, alkenylcarbonyloxy, alkynylcarbonyloxy, alkylsulfonyloxy, haloalkylsulfonyloxy, fluoroalkylsulfonyloxy, alkenylsulfonyloxy, alkynylsulfonyloxy, haloalkoxysulfonyloxy, fluoroalkoxysulfonyloxy, alkenyloxysulfonyloxy, alkynyloxysulfonyloxy, alkylsulfinyloxy, haloalkylsulfinyloxy, fluoroalkylsulfinyloxy, alkcnylsulfinyloxy, alkynylsulfinyloxy, alkoxysulfinyloxy, haloalkoxysulfinyloxy, fluoroalkoxysulfinyloxy, alkenyloxysulfinyloxy, alkynyloxysulfinyloxy, aminosulfinyloxy, amino, amido, aminosulfonyl, aminosulfinyl, cyano, nitro, azido, phosphinyl, phosphoryl, silyl, silyloxy, and any of said substituents bound to the heteroaryl group through an alkylene moiety (e.g. methylene).

The term “heteroarylene.” is art-recognized, and as used herein pertains to a bidentate moiety obtained by removing two hydrogen atoms of a heteroaryl ring, as defined above.

The term “heteroarylalkyl” or “heteroaralkyl” as used herein means a heteroaryl, as defined herein, appended to the parent molecular moiety through an alkyl group, as defined herein. Representative examples of heteroarylalkyl include, but are not limited to, pyridin-3-ylmethyl and 2-(thien-2-yl)ethyl.

The term “halo” or “halogen” means —Cl, —Br, —I or —F.

The term “haloalkyl” means an alkyl group, as defined herein, wherein at least one hydrogen is replaced with a halogen, as defined herein. Representative examples of haloalkyl include, but are not limited to, chloromethyl, 2-fluoroethyl, trifluoromethyl, pentafluoroethyl, and 2-chloro-3-fluoropentyl.

The term “fluoroalkyl” means an alkyl group, as defined herein, wherein all the hydrogens are replaced with fluorines.

The term “hydroxy” as used herein means an —OH group.

The term “alkoxy” as used herein means an alkyl group, as defined herein, appended to the parent molecular moiety through an oxygen atom. Representative examples of alkoxy include, but are not limited to, methoxy, ethoxy, propoxy, 2-propoxy, butoxy, tert-butoxy, pentyloxy, and hexyloxy. The terms “alkenyloxy”. “alkynyloxy”. “carbocyclyloxy”, and “heterocyclyloxy” are likewise defined.

The term “haloalkoxy” as used herein means an alkoxy group, as defined herein, wherein at least one hydrogen is replaced with a halogen, as defined herein. Representative examples of haloalkoxy include, but are not limited to, chloromethoxy, 2-fluoroethoxy, trifluoromethoxy, and pentafluoroethoxy. The term “fluoroalkyloxy” is likewise defined.

The term “aryloxy” as used herein means an aryl group, as defined herein, appended to the parent molecular moiety through an oxygen. The term “heteroaryloxy” as used herein means a heteroaryl group, as defined herein, appended to the parent molecular moiety through an oxygen. The terms “heteroaryloxy” is likewise defined.

The term “arylalkoxy” or “arylalkyloxy” as used herein means an arylalkyl group, as defined herein, appended to the parent molecular moiety through an oxygen. The term “heteroarylalkoxy” is likewise defined. Representative examples of aryloxy and heteroarylalkoxy include, but are not limited to, 2-chlorophenylmethoxy, 3-trifluoromethyl-phenylethoxy, and 2,3-dimethylpyridinylmethoxy.

The term “oxy” refers to a —O— group.

The term “carbonyl” as used herein means a —C(═O)— group.

The term “formyl” as used herein means a —C(═O)H group.

The term “alkylcarbonyl” as used herein means an alkyl group, as defined herein, appended to the parent molecular moiety through a carbonyl group, as defined herein. Representative examples of alkylcarbonyl include, but are not limited to, acetyl, 1-oxopropyl, 2,2-dimethyl-1-oxopropyl, 1-oxobutyl, and 1-oxopentyl. The terms “haloalkylcarbonyl”, “fluoroalkylcarbonyl”, “alkenylcarbonyl”, “alkynylcarbonyl”, “carbocyclylcarbonyl”, “heterocyclylcarbonyl”, “arylcarbonyl”. “aralkylcarbonyl”, “heteroarylcarbonyl”, and “heteroaralkylcarbonyl” are likewise defined.

The term “carboxy” as used herein means a —CO₂H group.

The term “alkoxycarbonyl” as used herein means an alkoxy group, as defined herein, appended to the parent molecular moiety through a carbonyl group, as defined herein. Representative examples of alkoxycarbonyl include, but are not limited to, methoxycarbonyl, ethoxycarbonyl, and tert-butoxycarbonyl. The terms “haloalkoxycarbonyl”, “fluoroalkoxycarbonyl”. “alkenyloxycarbonyl”, “alkynyloxycarbonyl”, “carbocyclyloxycarbonyl”, “heterocyclyloxycarbonyl”, “aryloxycarbonyl”, “aralkyloxycarbonyl”, “heteroaryloxycarbonyl”, and “heteroaralkyloxycarbonyl” are likewise defined.

The term “alkylcarbonyloxy” as used herein means an alkylcarbonyl group, as defined herein, appended to the parent molecular moiety through an oxygen atom. Representative examples of alkylcarbonyloxy include, but are not limited to, acetyloxy, ethylcarbonyloxy, and tert-butylcarbonyloxy. The terms “haloalkylcarbonyloxy”, “fluoroalkylcarbonyloxy”, “alkenylcarbonyloxy”, “alkynylcarbonyloxy”, “carbocyclylcarbonyloxy”. “heterocyclylcarbonyloxy”, “arylcarbonyloxy”, “aralkylcarbonyloxy”, “heteroarylcarbonyloxy”, and “heteroaralkylcarbonyloxy” are likewise defined.

The term “amino” as used herein refers to —NH₂ and substituted derivatives thereof wherein one or both of the hydrogens are independently replaced with substituents selected from the group consisting of alkyl, haloalkyl, fluoroalkyl, alkenyl, alkynyl, carbocyclyl, heterocyclyl, aryl, aralkyl, heteroaryl, heteroaralkyl, alkylcarbonyl, haloalkylcarbonyl, fluoroalkylcarbonyl, alkenylcarbonyl, alkynylcarbonyl, carbocyclylcarbonyl, heterocyclylcarbonyl, arylcarbonyl, aralkylcarbonyl, heteroarylcarnbonyl, heteroaralkylcarbonyl and the sulfonyl and sulfinyl groups defined above; or when both hydrogens together are replaced with an alkylene group (to form a ring which contains the nitrogen). Representative examples include, but are not limited to methylamino, acetylamino, and dimethylamino.

The term “amido” as used herein means an amino group, as defined herein, appended to the parent molecular moiety through a carbonyl.

The term “cyano” as used herein means a —C≡N group.

The term “nitro” as used herein means a —NO₂ group.

The abbreviations Me, Et, and Ph represent methyl, ethyl, and phenyl, respectively. A more comprehensive list of the abbreviations utilized by organic chemists of ordinary skill in the art appears in the first issue of each volume of the Journal of Organic Chemistry; this list is typically presented in a table entitled Standard List of Abbreviations.

As used herein, the term “administering” means providing a pharmaceutical agent or composition to a subject, and includes, but is not limited to, administering by a medical professional and self-administering.

As used herein, the phrase “pharmaceutically acceptable” refers to those agents, compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, the phrase “pharmaceutically-acceptable carrier” means a pharmaceutically-acceptable material, composition or vehicle, such as a liquid or solid filler, diluent, excipient, or solvent encapsulating material, involved in carrying or transporting an agent from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol mannitol and polyethylene glycol: (12) esters, such as ethyl oleate and ethyl laurate; (13) agar, (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions: (21) polyesters, polycarbonates and/or polyanhydrides; and (22) other non-toxic compatible substances employed in pharmaceutical formulations.

As used herein, the phrase “pharmaceutically-acceptable salts” refers to the relatively non-toxic, inorganic and organic salts of compounds.

As used herein, the term “subject” means a human or non-human animal selected for treatment or therapy.

As used herein, the phrase “subject suspected of having” means a subject exhibiting one or more clinical indicators of a disease or condition.

As used herein, the phrase “therapeutic effect” refers to a local or systemic effect in animals, particularly mammals, and more particularly humans, caused by an agent. The phrases “therapeutically-effective amount” and “effective amount” mean the amount of an agent that produces some desired effect in at least a sub-population of cells. A therapeutically effective amount includes an amount of an agent that produces some desired local or systemic effect at a reasonable benefit/risk ratio applicable to any treatment. For example, certain agents used in the methods of the present disclosure may be administered in a sufficient amount to produce a reasonable benefit/risk ratio applicable to such treatment.

As used herein, the term “treating” a disease in a subject or “treating” a subject having or suspected of having a disease refers to subjecting the subject to a pharmaceutical treatment, e.g., the administration of an agent, such that at least one symptom of the disease is decreased or prevented from worsening.

As used herein, “HIV” refers to any virus that can infect a host cell of a subject through activation of the gp120 envelope glycoproteins (Env gps). “HIV” encompasses all strains of HIV-1 and HIV-2. Compounds of the present disclosure, however, are also useful to treat other immunodeficiency viruses expressing gp120 such as some strains of simian immunodeficiency virus SIV.

As used herein “gp120” refers to the gp120 envelope glycoprotein, and “Env gps” refers to the complete envelope glycoprotein complex which is a trimer of three gp120s and three gp41s.

As used herein, the term “activating” when referring to gp120 envelope glycoprotein means the association of a natural or non-natural ligand with the conserved domain of gp 120 that induces a conformational change that activates binding to the chemokine receptors CCR5 or CXCR4. Examples of natural ligands include CD4 and sCD4. Examples of non-natural ligands include compounds of the present disclosure as well as NBD-556 and NBD-557.

As used herein “activated intermediate” refers to the gp120 envelope glycoprotein in bound form with CD4, sCD4, or compounds of the present disclosure.

As used herein, the term “contacting” when used in the context of compounds of the present disclosure and gp120, refers to the process of supplying compounds of the present disclosure to the HIV envelope glycoprotein either in vitro or in vivo in order effect the selective binding of the compounds of the present disclosure to the conserved Phe43 binding pocket of gp120. For the in vitro process, this can entail simply adding an amount of a stock solution of one or more compounds of the present disclosure to a solution preparation of gp120. For an in vive process, “selective binding” involves making compounds of the present disclosure available to interact with gp120 in a host organism, wherein the compounds of the disclosure exhibit a selectivity for the conserved domain of gp120 that define the Phe43 cavity. Making the compounds available to interact with gp120 in the host organism can be achieved by oral administration, intravenously, peritoncally, mucosally, intramuscularly, and other methods familiar to one of ordinary skill in the art.

As used herein, the term “inhibiting” when referring to transmission means reducing the rate of or blocking the process that allows fusion of the viral glycoprotein gp120 to a host cell and introduction of the viral core into the host cell. In this regard, inhibiting transmission includes prophylactic measures to prevent viral spread from one host organism to another. When referring to progression, “inhibiting” refers to the treatment of an already infected organism and preventing further viral invasion within the same organism by blocking the process that allows fusion of the viral glycoprotein gp120 and introduction of viral core into additional host cells of the organism.

As used herein, the term “antibody” refers to a protein that includes at least one immunoglobulin variable domain or immunoglobulin variable domain sequence. For example, an antibody can include a heavy (H) chain variable region (abbreviated herein as VH), and a light (L) chain variable region (abbreviated herein as VL). In another example, an antibody includes two heavy (H) chain variable regions and two light (L) chain variable regions. The term “antibody” encompasses antigen-binding fragments of antibodies (e.g., single chain antibodies, Fab fragments, F(ab)₂, a Fd fragment, a Fv fragments, and dAb fragments) as well as complete antibodies.

The term “conformation” or “conformational state” of a protein refers generally to the range of structures that a protein may adopt at any instant in time One of skill in the art will recognize that determinants of conformation or conformational state include a protein's primary structure as reflected in a protein's amino acid sequence (including modified amino acids) and the environment surrounding the protein. The conformation or conformational state of a protein also relates to structural features such as protein secondary structures (e.g., a-helix, β-sheet, among others), tertiary structure (e.g., the three dimensional folding of a polypeptide chain), and quaternary structure (e.g., interactions of a polypeptide chain with other protein subunits). Post-translational and other modifications to a polypeptide chain such as ligand binding, phosphorylation, sulfation, glycosylation, or attachments of hydrophobic groups, among others, can influence the conformation of a protein. Furthermore, environmental factors, such as pH, salt concentration, ionic strength, and osmolality of the surrounding solution, and interaction with other proteins and co-factors, among others, can affect protein conformation. The conformational state of a protein may be determined by either functional assay for activity or binding to another molecule or by means of physical methods such as X-ray crystallography, NMR, or spin labeling, among other methods. For a general discussion of protein conformation and conformational states, one is referred to Cantor and Schimmel. Biophysical Chemistry, Part I: The Conformation of Biological. Macromolecules, W.H. Freeman and Company, 1980, and Creighton, Proteins: Structures and Molecular Properties, W.H. Freeman and Company, 1993. A “specific conformational state” is any subset of the range of conformations or conformational states that a protein may adopt.

### Exemplary Compounds Used in Method of the Disclosure

In certain embodiments, the disclosure relates to methods, wherein the method involves the use of a compound. The methods of the disclosure are described in detail in this application. In certain embodiments, the disclosure relates to methods involving compounds described in, for example, International Patent Application Publication No. WO13/090696, which is hereby incorporated by reference in its entirety. In certain embodiments, the compound is (+)-DMJ-I-228 or (+)-DMJ-II-121.

In certain embodiments, the disclosure relates to methods of using a compound of Formula VII

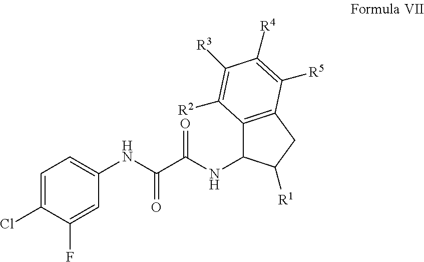

or a pharmaceutically acceptable salt or solvate thereof,
wherein, independently for each occurrence,

R¹ is selected from the group consisting of optionally substituted amino,

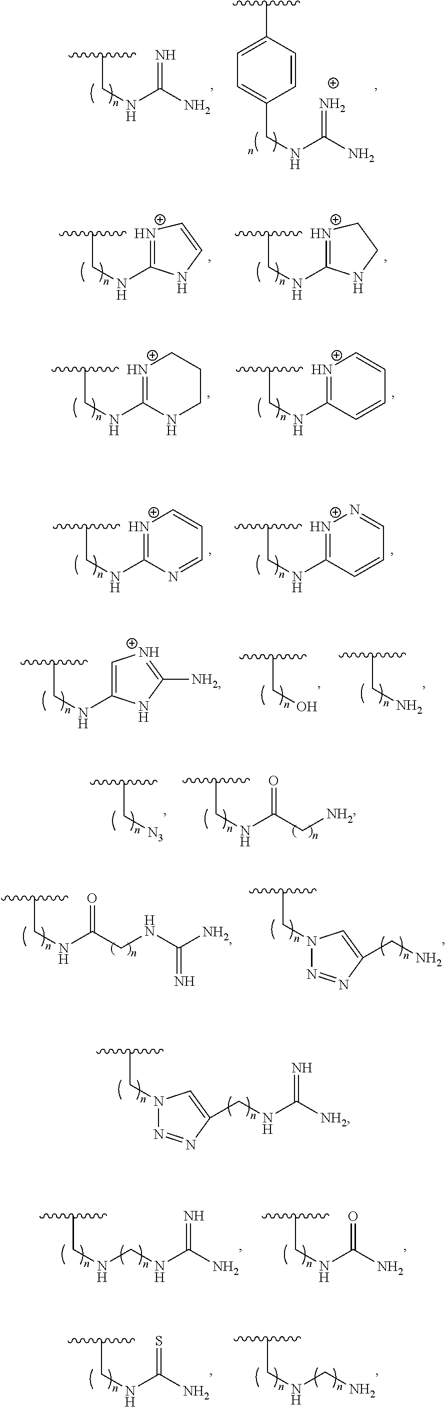

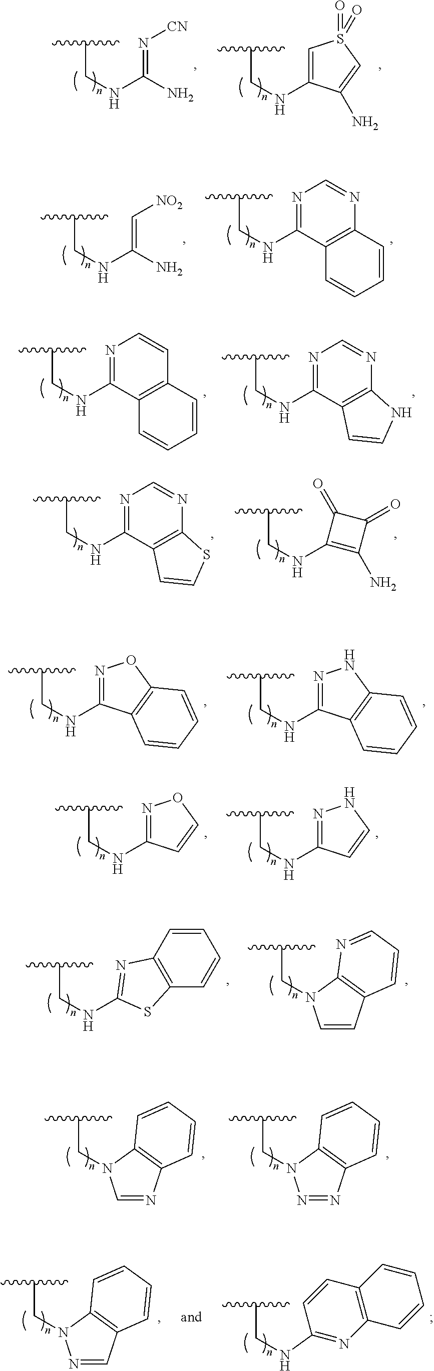

R² is —H, optionally substituted alkyl, optionally substituted alkenyl, optionally substituted alkynyl, optionally substituted aryl, optionally substituted heteroaryl hydroxy, optionally substituted alkoxy, optionally substituted amino, or halo;

R³ is —H, optionally substituted alkyl, optionally substituted alkenyl, optionally substituted alkynyl, optionally substituted aryl, optionally substituted heteroaryl, hydroxy, optionally substituted alkoxy, optionally substituted amino, or halo;

R⁴ is —H, optionally substituted alkyl, optionally substituted alkenyl, optionally substituted alkynyl, optionally substituted aryl, optionally substituted heteroaryl, hydroxy, optionally substituted alkoxy, optionally substituted amino, or halo;

R⁵ is —H, optionally substituted alkyl, optionally substituted alkenyl, optionally substituted alkynyl, optionally substituted aryl, optionally substituted heteroaryl, hydroxy, optionally substituted alkoxy, optionally substituted amino, or halo; and

n is 0, 1, 2, 3, 4, or 5.

Particularly preferred compounds of Formula VII include (+)-DMJ-I-228 and (+)-DMJ-II-121.

In certain embodiments, the disclosure relates to methods of using a compound of Formula I

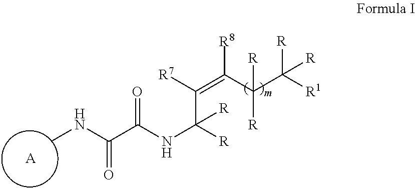

or a pharmaceutically acceptable salt or solvate thereof,
wherein, independently for each occurrence,

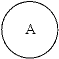

is optionally substituted aryl or heteroaryl;

R¹ is selected from the group consisting of optionally substituted amino,

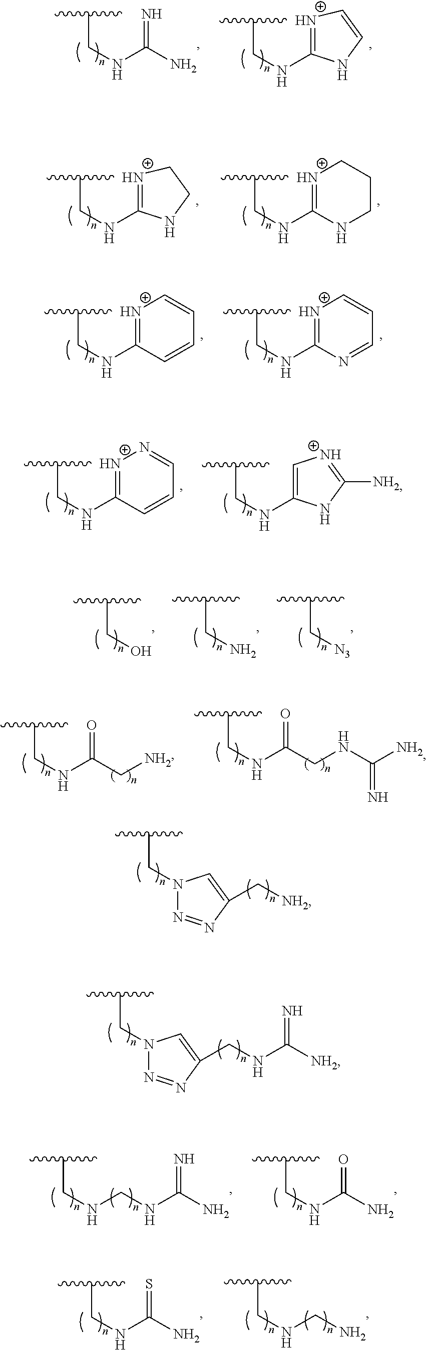

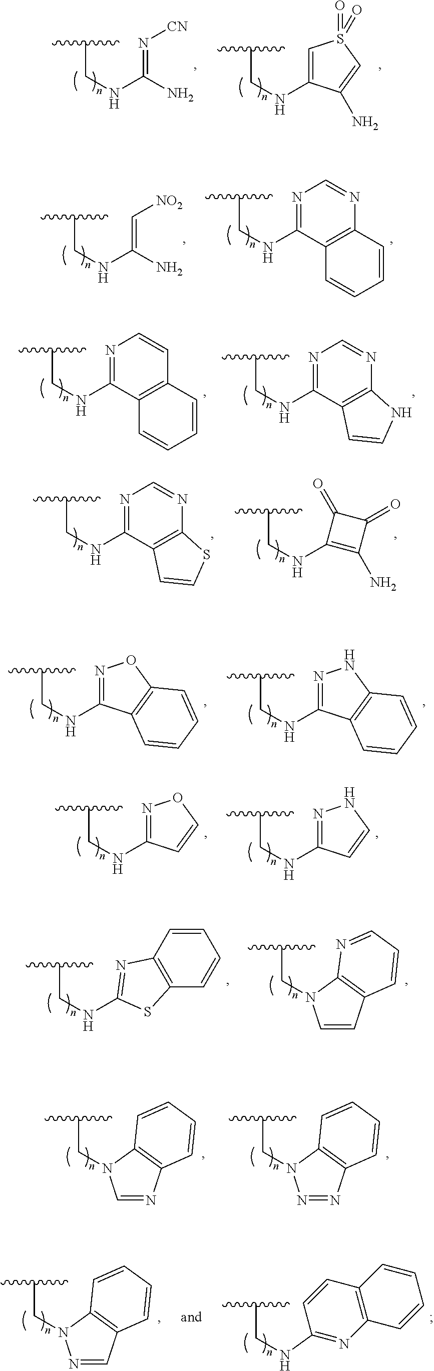

R⁷ is —H, optionally substituted alkyl, optionally substituted alkenyl, optionally substituted alkynyl, optionally substituted aryl, optionally substituted heteroaryl, hydroxy, optionally substituted alkoxy, optionally substituted amino, or halo;

R⁸ is —H, optionally substituted alkyl, optionally substituted alkenyl, optionally substituted alkynyl, optionally substituted aryl, optionally substituted heteroaryl, hydroxy, optionally substituted alkoxy, optionally substituted amino, or halo;
or, R⁷ and R⁸, taken together, form an optionally substituted five-membered heteroaryl ring or an optionally substituted six-membered aryl or heteroaryl ring;
m is 1, 2, 3, or 4;

R is —H, optionally substituted alkyl, hydroxy, optionally substituted alkoxy, optionally substituted amino, or halo; and
n is 0, 1, 2, 3, 4, or 5.

In certain embodiments, the disclosure relates to methods of using a compound of Formula II

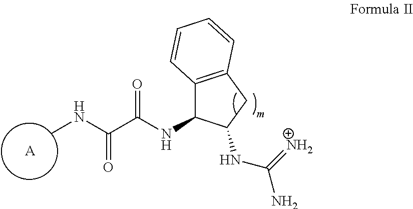

or a pharmaceutically acceptable salt or solvate thereof,
wherein, independently for each occurrence,

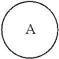

is optionally substituted aryl or optionally substituted heteroaryl, and

m is 1, 2, 3, or 4.

In preferred embodiments, m is 1 or 2. In particularly preferred embodiments, m is 1.

In certain embodiments, the compound of Formula II is selected from the group consisting of

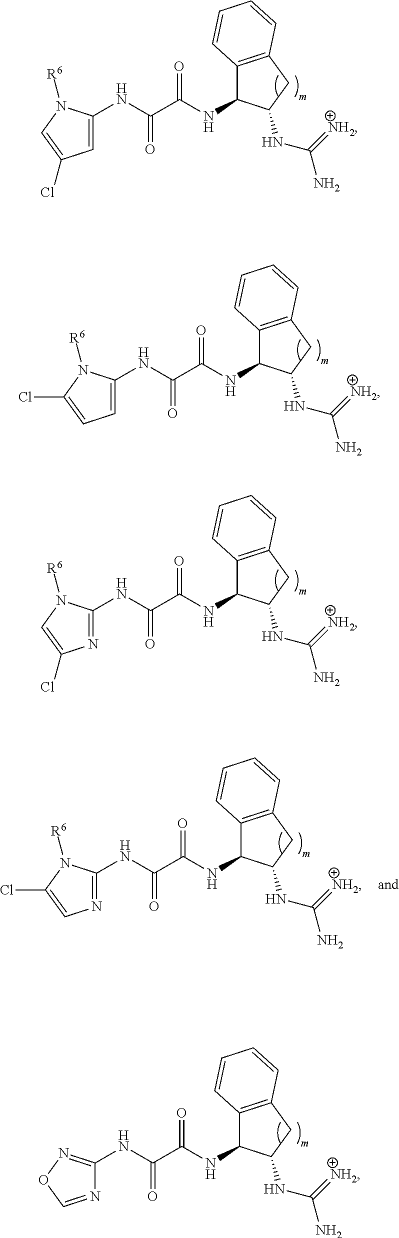

wherein,

R⁶ is selected from the group consisting of —H, optionally substituted alkyl, optionally substituted aralkyl, optionally substituted heteroaralkyl, optionally substituted alkylcarbonyl, optionally substituted cycloalkylsulfonyl, and optionally substituted alkylsulfonyl.

Preferably, R⁶ is selected from the group consisting of

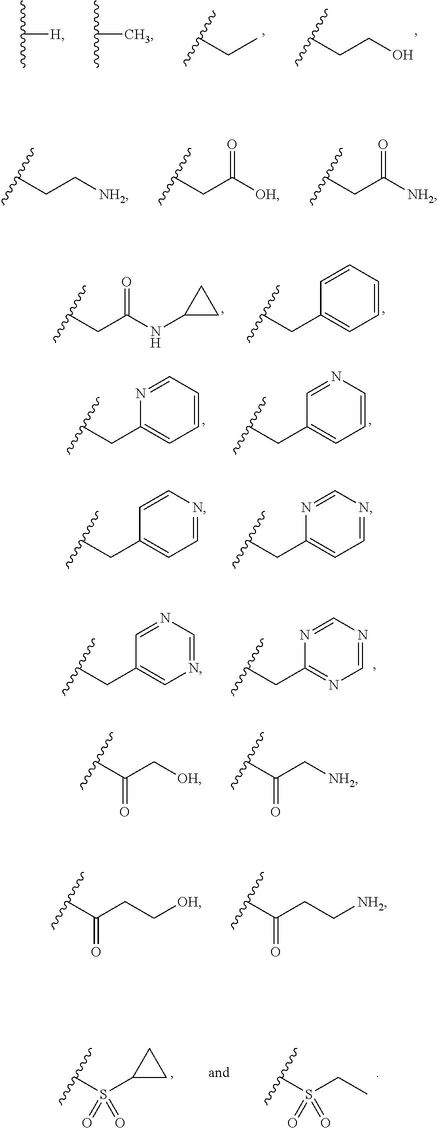

In certain embodiments, the disclosure relates to methods of using a compound of Formula III

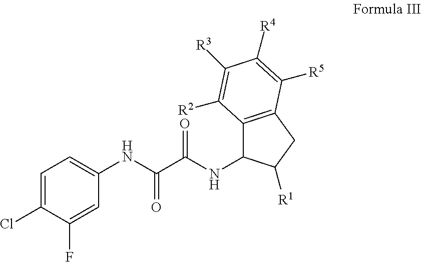

or a pharmaceutically acceptable salt or solvate thereof,
wherein, independently for each occurrence.

R¹ is selected from the group consisting of optionally substituted amino,

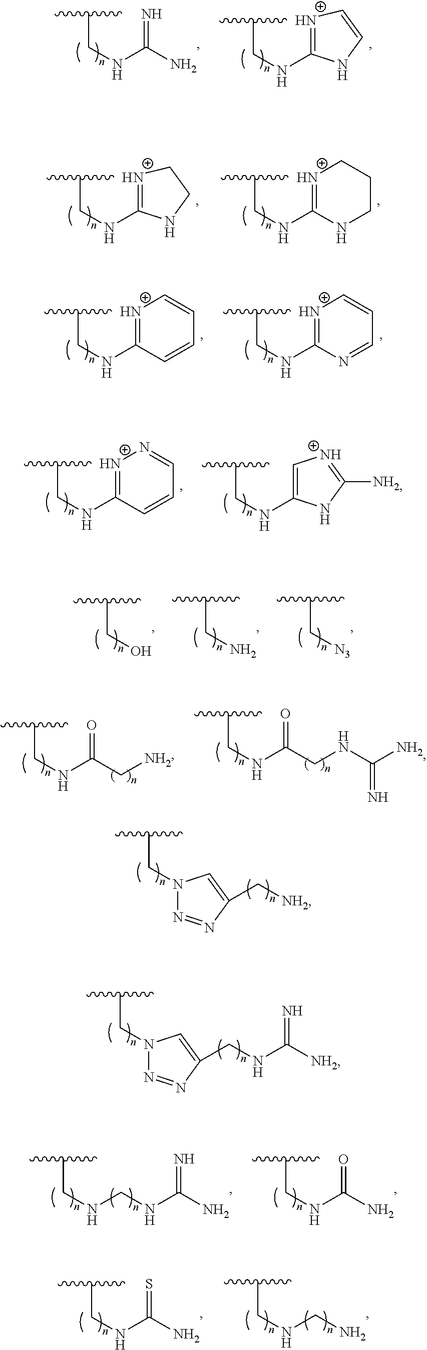

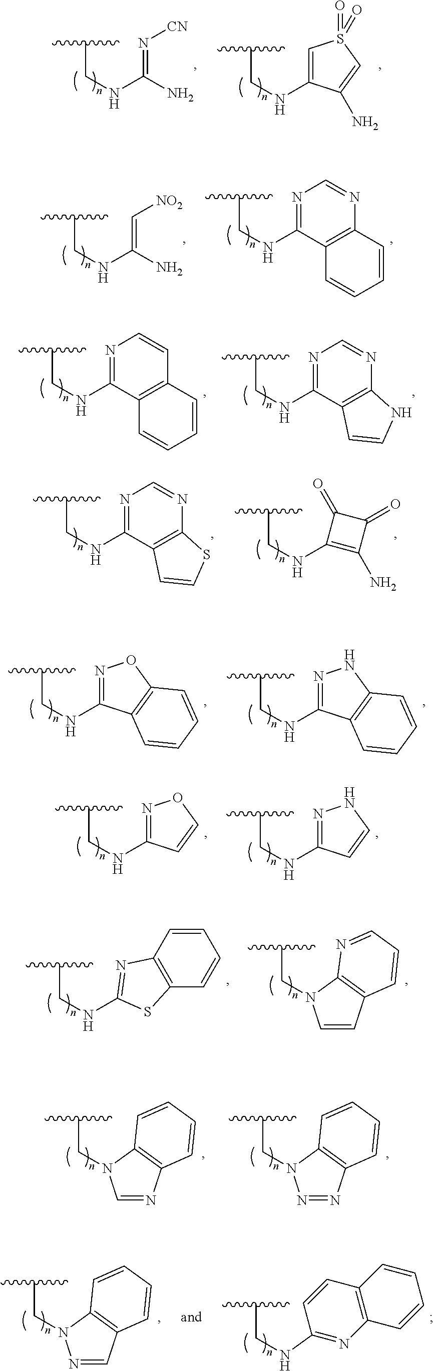

R² is —H, optionally substituted alkyl, optionally substituted alkenyl, optionally substituted alkynyl, optionally substituted aryl, optionally substituted heteroaryl, hydroxy, optionally substituted alkoxy, optionally substituted amino, or halo;

R³ is —H, optionally substituted alkyl, optionally substituted alkenyl, optionally substituted alkynyl, optionally substituted aryl, optionally substituted heteroaryl, hydroxy, optionally substituted alkoxy, optionally substituted amino, or halo;

R⁴ is —H, optionally substituted alkyl, optionally substituted alkenyl, optionally substituted alkynyl, optionally substituted aryl, optionally substituted heteroaryl, hydroxy, optionally substituted alkoxy, optionally substituted amino, or halo;

R⁵ is —H, optionally substituted alkyl, optionally substituted alkenyl, optionally substituted alkynyl, optionally substituted aryl, optionally substituted heteroaryl, hydroxy, optionally substituted alkoxy, optionally substituted amino, or halo; and

n is 0, 1, 2, 3, 4, or 5.

In certain embodiments, the disclosure relates to methods of using a compound of Formula IV

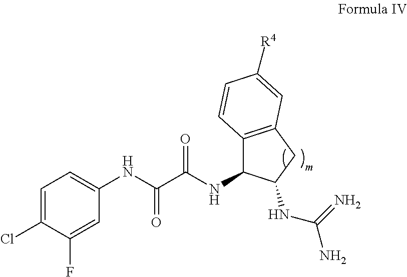

or a pharmaceutically acceptable salt or solvate thereof.
wherein, independently for each occurrence.

R⁴ is selected from the group consisting of halo, hydroxy, thio, optionally substituted alkylsulfonamido, optionally substituted cycloalkylsulfonamido, optionally substituted amino, optionally substituted amido, optionally substituted heterocyclyl, optionally substituted heteroaryl, and optionally substituted aryl; and

m is 1, 2, 3, or 4.

Preferably, m is 1 or 2. In particularly preferred embodiments, m is 1.

In certain embodiments, the disclosure relates to methods of using any one of the aforementioned compounds, wherein R⁴ is selected from the group consisting of

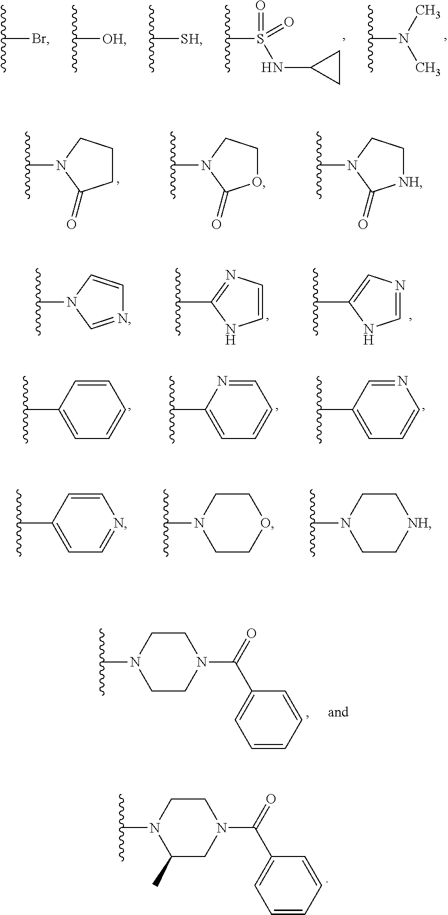

In certain embodiments, the disclosure relates to methods of using a compound of Formula V

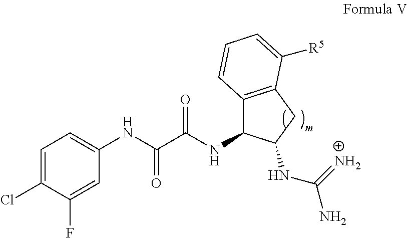

or a pharmaceutically acceptable salt or solvate thereof,
wherein, independently for each occurrence,

R⁵ is selected from the group consisting of halo, hydroxy, thio, optionally substituted alkylsulfonamido, optionally substituted cycloalkylsulfonamido, optionally substituted amino, optionally substituted amido, optionally substituted heterocyclyl, optionally substituted heteroaryl, and optionally substituted aryl; and

m is 1, 2, 3, or 4.

Preferably, m is 1 or 2. In particularly preferred embodiments, m is 1.

For compounds of Formula V, R⁵ is preferably selected from the group consisting of

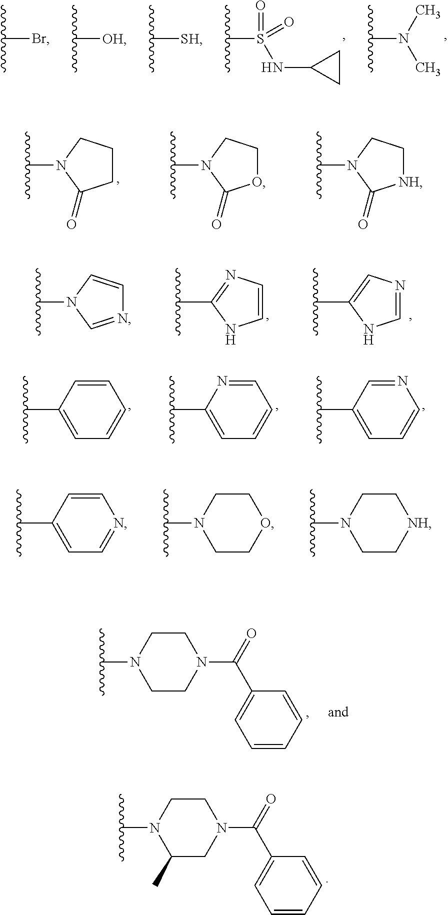

In certain embodiments, the disclosure relates to methods of using a compound selected from the group consisting of

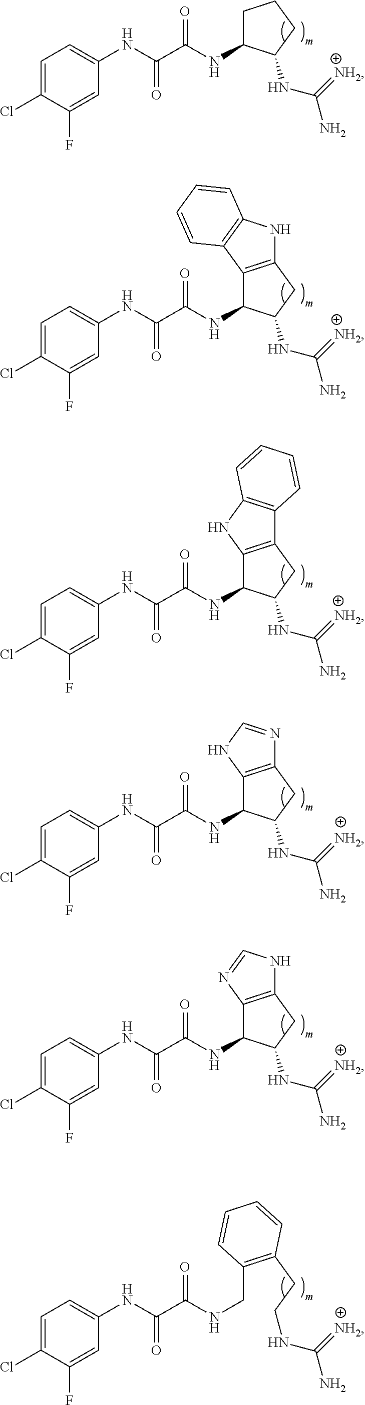

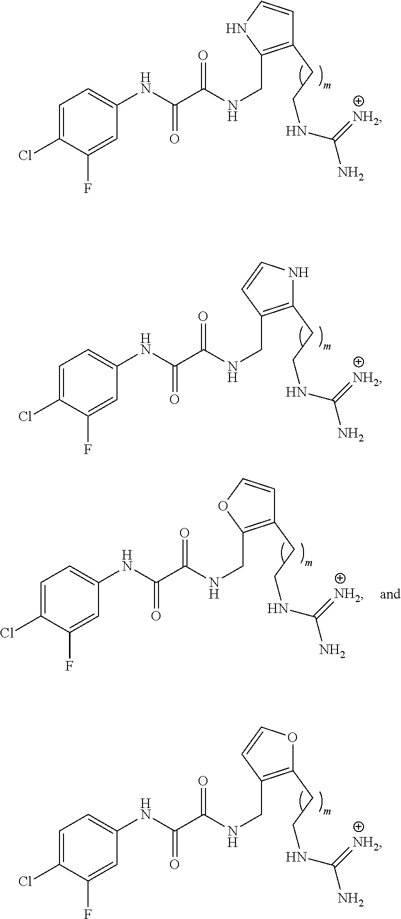

wherein m is 1, 2, 3, or 4.

Preferably, m is 1 or 2. In particularly preferred embodiments, m is 1.

In certain embodiments, the disclosure relates to methods of using a compound of Formula VI

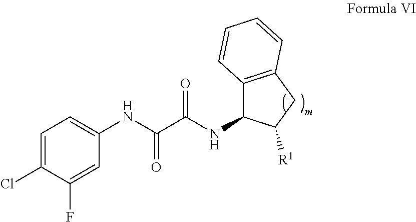

or a pharmaceutically acceptable salt or solvate thereof,
wherein, independently for each occurrence,

R¹ is selected from the group consisting of optionally substituted amino,

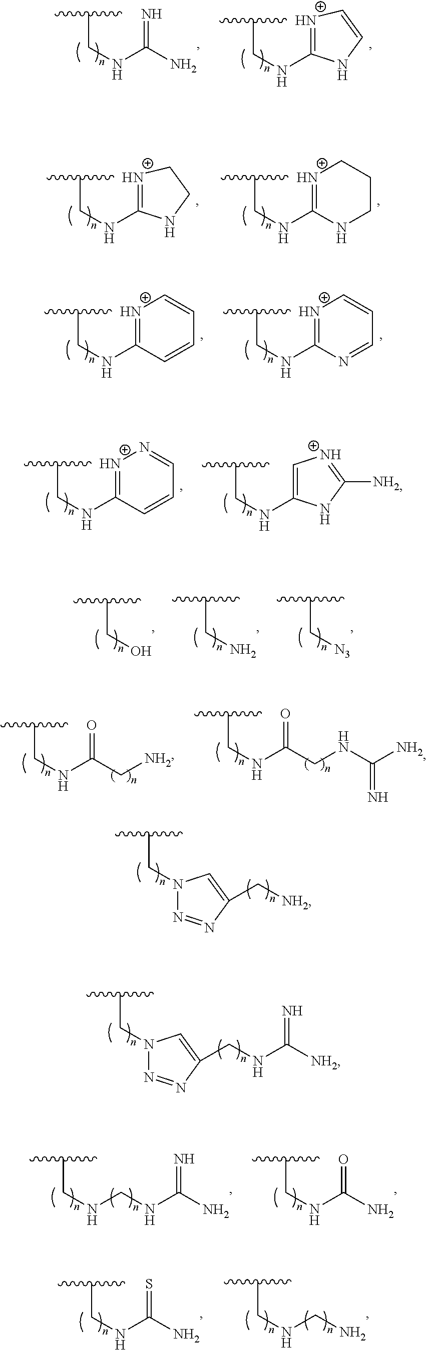

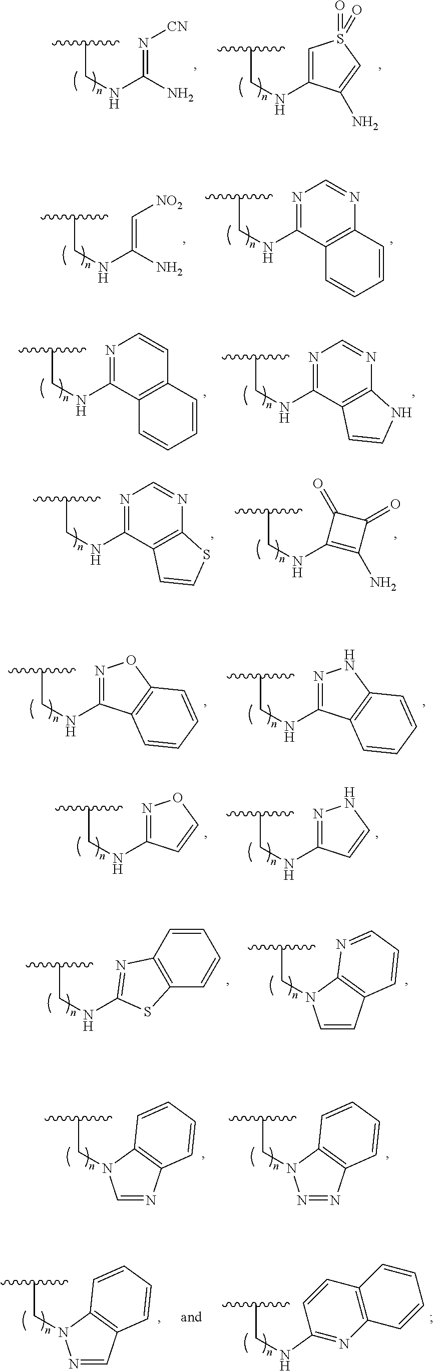

m is 1, 2, 3, or 4, preferably 1 or 2, and even more preferably, m is 1; and

n is 0, 1, 2, 3, 4, or 5, preferably 0, 1, or 2, and even more preferably, n is 1.

In certain embodiments, the disclosure relates to methods of using a compound of Formula VIII

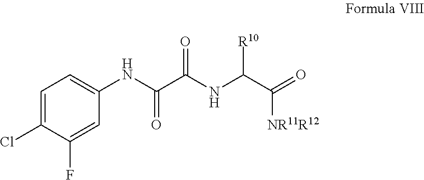

or a pharmaceutically acceptable salt or solvate thereof,
wherein, independently for each occurrence.

R¹⁰ is optionally substituted aryl, optionally substituted heteroaryl, optionally substituted cycloalkyl, optionally substituted heterocycloalkyl, optionally substituted alkyl, or optionally substituted alkenyl;

R¹¹ is —H, optionally substituted alkyl, optionally substituted alkenyl, optionally substituted alkynyl, optionally substituted aryl, or optionally substituted heteroaryl; and

R¹² is optionally substituted alkyl, optionally substituted alkenyl, optionally substituted alkynyl, optionally substituted aryl, or optionally substituted heteroaryl.

In certain embodiments, the disclosure relates to methods of using any one of the compounds of Formula VIII, wherein R¹⁰ is selected from the group consisting of:

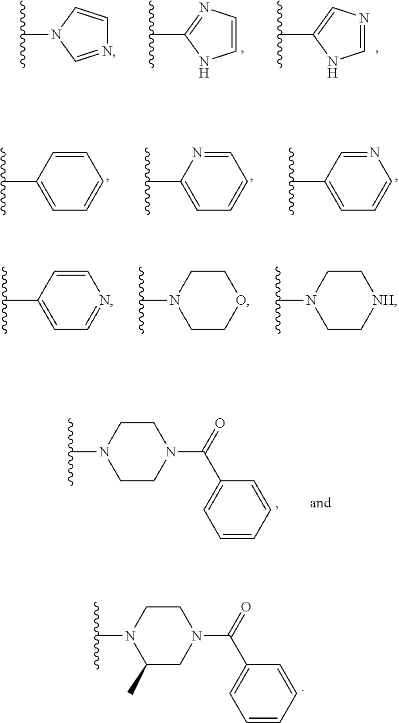

In certain embodiments, the disclosure relates to methods of using any one of the compounds of Formula VIII, wherein R¹¹ is —H or optionally substituted alkyl.

In certain embodiments, the disclosure relates to methods of using any one of the compounds of Formula VIII, wherein R¹² is optionally substituted alkyl.

In certain embodiments, the compound of Formula VIII has the following structure:

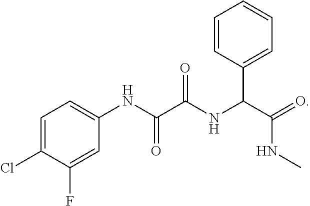 [SMILES: CNC(=O)C(NC(=O)C(=O)Nc1ccc(Cl)c(F)c1)c1ccccc1]

In certain embodiments, the disclosure relates to methods of using a compound selected from the group consisting of

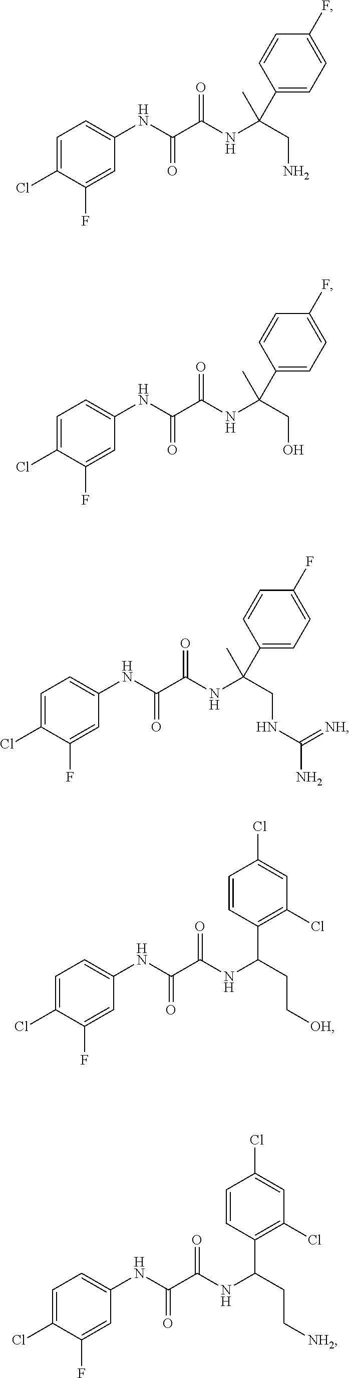

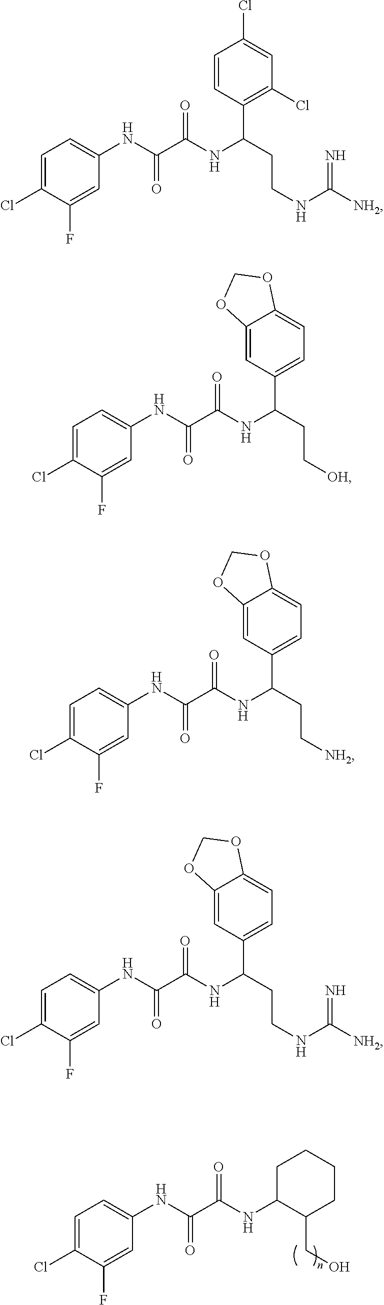

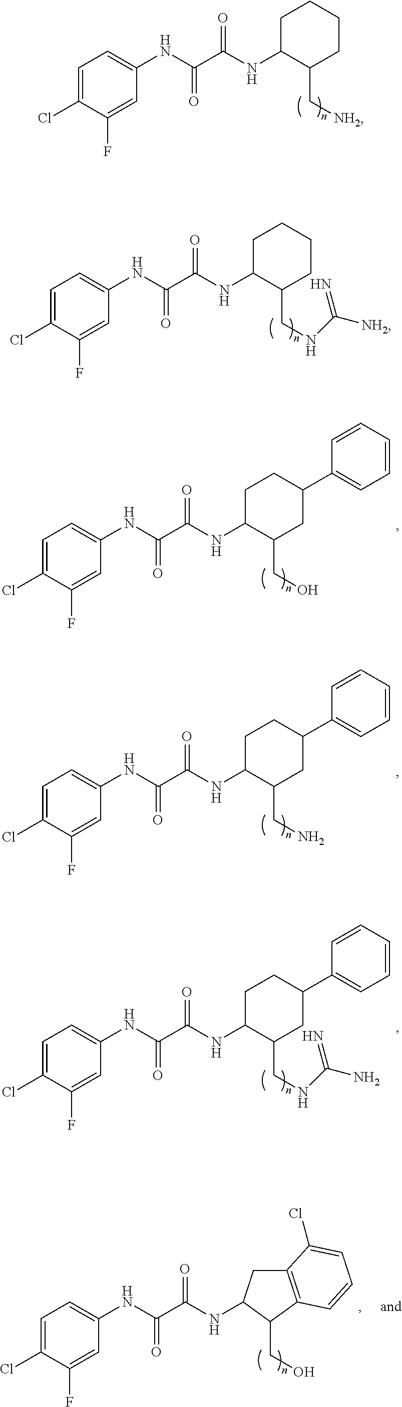

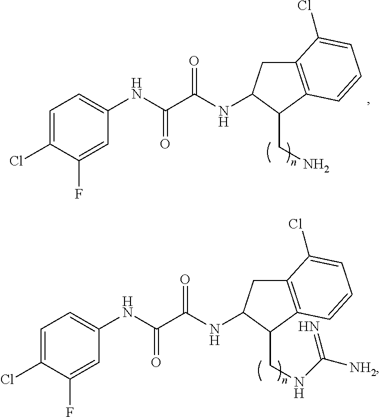

wherein n is 0, 1, 2, 3, 4, or 5, preferably 0, 1, or 2, and even more preferably n is 1.

In certain embodiments, the disclosure relates to methods of using any one of the aforementioned compounds, wherein the compound is a single enantiomer.

### Exemplary Methods

In certain embodiments, the disclosure relates to methods of generating a protein binding domain that specifically binds to gp120 in a specific conformational state, the method comprising the steps of:
a) contacting gp120 or a fragment thereof with a compound, wherein the compound is a compound of Formula VII, Formula I, Formula II, Formula III, Formula IV, Formula V, Formula VI, or Formula VIII, thereby forming gp120 in the specific conformational state; and
b) generating antibodies to the gp120 or the fragment thereof in the specific conformation state, wherein the compound is optionally bound to the gp120 or the fragment thereof in the specific conformational state.

In certain embodiments, the disclosure relates to any one of the aforementioned methods, wherein the protein binding domain is an antibody.

In certain embodiments, the disclosure relates to methods of neutralizing HIV-1, the method comprising the step of:
contacting HIV-1 with an effective amount of a compound of Formula VII, Formula I, Formula II, Formula III, Formula IV, Formula V, Formula VI, or Formula VIII, thereby forming HIV-1 having gp120 in a specific conformational state; and
contacting the HIV-1 in the specific conformational state with an antibody, wherein the compound is optionally bound to the HIV-1 in the specific conformational state.

In certain embodiments, the disclosure relates to methods of treating or preventing HIV infection, the method comprising the step of:
administering to a subject in need thereof, a therapeutically effective amount of an antibody; and
co-administering to the subject an effective amount of a compound of Formula VII, Formula I, Formula II, Formula III, Formula IV, Formula V, Formula VI, or Formula VIII.

In certain embodiments, the disclosure relates to any one of the aforementioned methods, wherein the antibody is a monoclonal antibody.

In certain embodiments, the disclosure relates to any one of the aforementioned methods, wherein the antibody is a monoclonal antibody directed against CD4-induced (CD4i) epitopes or the V3 region.

In certain embodiments, the disclosure relates to any one of the aforementioned methods, wherein the antibody is an anti-gp120 antibody.

In certain embodiments, the disclosure relates to any one of the aforementioned methods, wherein the HIV is primary HIV-1 JR-FL or a transmitted/founder virus.

In certain embodiments, the disclosure relates to any one of the aforementioned methods, wherein the compound is a compound of Formula VII.

In certain embodiments, the disclosure relates to any one of the aforementioned methods, wherein the compound is

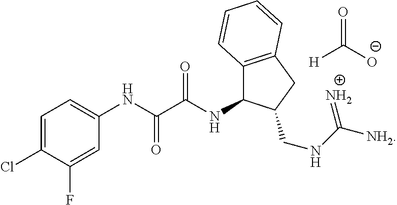

In certain embodiments, the disclosure relates to any one of the aforementioned methods, wherein the compound is

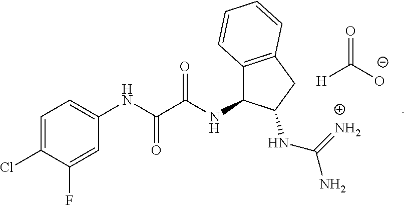

For example, the disclosure relates to methods of immunizing an animal. In certain embodiments, antibodies may be generated that specifically bind to a conformational epitope of an active conformational state of gp120 by administering to a subject gp120 in the presence of any one of the aforementioned compounds.

For the immunization of an animal with gp120, the gp120 may be produced and purified using conventional methods that may employ expressing a recombinant form of the gp120 in a host cell, and purifying the gp120 using affinity chromatography and/or antibody-based methods. In particular embodiments, the baculovirus/Sf-9 system may be employed for expression, although other expression systems (e.g., bacterial, yeast or mammalian cell systems) may also be used. Exemplary methods for expressing and purifying gp120s are described in the art. A gp120 may also be reconstituted in phospholipid vesicles. Likewise, methods for reconstituting an active gp120 in phospholipid vesicles are known. In certain cases, the gp120 and phospholipids may be reconstituted at high density (e.g., 1 mg receptor per mg of phospholipid). In particular embodiments, the phospholipids vesicles may be tested to confirm that the gp120 is active. In many cases, a gp120 may be present in the phospholipid vesicle in both orientations (in the normal orientation, and in the “upside down” orientation in which the intracellular loops are on the outside of the vesicle). Other immunization methods with gp120 include, without limitation, the use of complete cells expressing a gp120, vaccination with a nucleic acid sequence encoding a gp120 (e.g. DNA vaccination), immunization with viruses or virus like particles expressing a gp120, amongst others.

Any suitable animal, e.g., a warm-blooded animal, in particular a mammal such as a rabbit, mouse, rat, camel, sheep, cow, shark, or pig or a bird such as a chicken or turkey, may be immunized using any of the techniques well known in the art suitable for generating an immune response.

The screening for antibodies, as a non-limiting example, specifically binding to a conformational epitope of a functional conformational state of said gp120 may for example be performed by screening a set, collection or library of cells that express heavy chain antibodies on their surface, or bacteriophages, or by screening of a (naive or immune) library of peptide sequences, which may all be performed in a manner known per se, and which method may optionally further comprise one or more other suitable steps, such as, for example and without limitation, a step of affinity maturation, a step of expressing the desired amino acid sequence, a step of screening for binding and/or for activity against the desired antigen (in this case, the gp120), a step of determining the desired amino acid sequence or nucleotide sequence, a step of introducing one or more humanizing substitutions, a step of formatting in a suitable multivalent and/or multispecific format, a step of screening for the desired biological and/or physiological properties (i.e. using a suitable assay known in the art), and/or any combination of one or more of such steps, in any suitable order.

### Exemplary Complexes

In certain embodiments, the disclosure relates to a complex comprising (i) a compound of Formula VII, Formula I, Formula II, Formula III, Formula IV, Formula V, Formula VI, or Formula VIII, (ii) gp120 in a functional conformational state, and (iii) optionally, an antibody.

In certain embodiments, the disclosure relates to any one of the aforementioned complexes, wherein said complex is in a solubilized form or immobilized to a solid support.

In certain embodiments, the disclosure relates to any one of the aforementioned complexes, wherein the compound is a compound of Formula VII.

In certain embodiments, the disclosure relates to any one of the aforementioned complexes, wherein the compound is

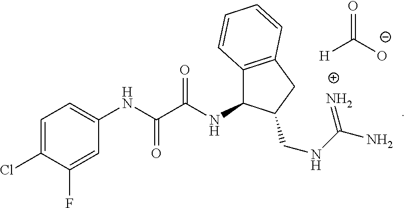

In certain embodiments, the disclosure relates to any one of the aforementioned complexes, wherein the compound is

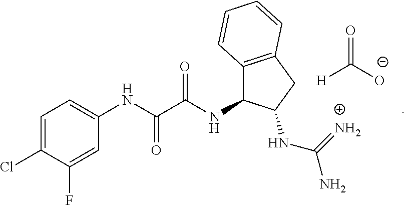

In certain embodiments, the disclosure relates to any one of the aforementioned complexes, wherein the antibody is a monoclonal antibody.

In certain embodiments, the disclosure relates to any one of the aforementioned complexes, wherein the antibody is a monoclonal antibody directed against CD4-induced (CD4i) epitopes or the V3 region.

In certain embodiments, the disclosure relates to any one of the aforementioned complexes, wherein the antibody is an anti-gp120 antibody.

### Exemplary Pharmaceutical Compositions

While it is possible for compounds of the disclosure to be administered as the raw chemical, it is also possible to present them as a pharmaceutical formulation. Accordingly, the disclosure provides a pharmaceutical formulation comprising a compound or a pharmaceutically acceptable salt, prodrug or solvate thereof, together with one or more pharmaceutically acceptable carriers thereof and optionally one or more other therapeutic ingredients. The carrier(s) must be “acceptable” in the sense of being compatible with the other ingredients of the formulation and not deleterious to the recipient thereof. Proper formulation is dependent upon the route of administration chosen. Any of the well-known techniques, carriers, and excipients can be used as suitable and as understood in the art; e.g., in Remington's Pharmaceutical Sciences. The pharmaceutical compositions of the disclosure can be manufactured in a manner that is itself known, e.g., by means of conventional mixing, dissolving, granulating, dragee-making, levigating, emulsifying, encapsulating, entrapping or compression processes, for example.

The formulations include those suitable for oral, parenteral (including subcutaneous, intradermal, intramuscular, intravenous, intraarticular, and intramedullary), intraperitoneal, transmucosal, transdermal, rectal and topical (including dermal, buccal, sublingual and intraocular) administration although the most suitable route depends upon for example the condition and disorder of the recipient. The formulations can conveniently be presented in unit dosage form and can be prepared by any of the methods well known in the art. All methods include the step of bringing into association a compound of the disclosure or a pharmaceutically acceptable salt, prodrug or solvate thereof (“active ingredient”) with the carrier which constitutes one or more accessory ingredients. In general, the formulations are prepared by uniformly and intimately bringing into association the active ingredient with liquid carriers or finely divided solid carriers or both and then, if necessary, shaping the product into the desired formulation.

Formulations of the disclosure suitable for oral administration can be presented as discrete units such as capsules, cachets or tablets each containing a predetermined amount of the active ingredient; as a powder or granules; as a solution or a suspension in an aqueous liquid or a non-aqueous liquid; or as an oil-in-water liquid emulsion or a water-in-oil liquid emulsion. The active ingredient can also be presented as a bolus, electuary or paste.

Pharmaceutical preparations which can be used orally include tablets, push-fit capsules made of gelatin, as well as soft, sealed capsules made of gelatin and a plasticizer, such as glycerol or sorbitol. Tablets can be made by compression or molding, optionally with one or more accessory ingredients. Compressed tablets can be prepared by compressing in a suitable machine the active ingredient in a free-flowing form such as a powder or granules, optionally mixed with binders, inert diluents, or lubricating, surfaceactive or dispersing agents. Molded tablets can be made by molding in a suitable machine a mixture of the powdered compound moistened with an inert liquid diluent. The tablets can optionally be coated or scored and can be formulated so as to provide slow or controlled release of the active ingredient therein. All formulations for oral administration should be in dosages suitable for such administration. The push-fit capsules can contain the active ingredients in admixture with filler such as lactose, binders such as starches, and/or lubricants such as tale or magnesium stearate and, optionally, stabilizers. In soft capsules, the active compounds can be dissolved or suspended in suitable liquids, such as fatty oils, liquid paraffin, or liquid polyethylene glycols. In addition, stabilizers can be added. Dragee cores are provided with suitable coatings. For this purpose, concentrated sugar solutions can be used, which can optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments can be added to the tablets or dragee coatings for identification or to characterize different combinations of active compound doses.

The compounds can be formulated for parenteral administration by injection, e.g., by bolus injection or continuous infusion. Formulations for injection can be presented in unit dosage form, e.g., in ampoules or in multi-dose containers, with an added preservative. The compositions can take such forms as suspensions, solutions or emulsions in oily or aqueous vehicles, and can contain formulatory agents such as suspending, stabilizing and/or dispersing agents. The formulations can be presented in unit-dose or multi-dose containers, for example sealed ampoules and vials, and can be stored in powder form or in a freeze-dried (lyophilized) condition requiring only the addition of the sterile liquid carrier, for example, saline or sterile pyrogen-free water, immediately prior to use. Extemporaneous injection solutions and suspensions can be prepared from sterile powders, granules and tablets of the kind previously described.

Formulations for parenteral administration include aqueous and non-aqueous (oily) sterile injection solutions of the active compounds which can contain antioxidants, buffers, bacteriostats and solutes which render the formulation isotonic with the blood of the intended recipient: and aqueous and non-aqueous sterile suspensions which can include suspending agents and thickening agents. Suitable lipophilic solvents or vehicles include fatty oils such as sesame oil, or synthetic fatty acid esters, such as ethyl oleate or triglycerides, or liposomes. Aqueous injection suspensions can contain substances which increase the viscosity of the suspension, such as sodium carboxymethyl cellulose, sorbitol, or dextran. Optionally, the suspension can also contain suitable stabilizers or agents which increase the solubility of the compounds to allow for the preparation of highly concentrated solutions.

In addition to the formulations described previously, the compounds of the disclosure can also be formulated as a depot preparation. Such long acting formulations can be administered by implantation (for example subcutaneously or intramuscularly) or by intramuscular injection. Thus, for example, the compounds can be formulated with suitable polymeric or hydrophobic materials (for example as an emulsion in an acceptable oil) or ion exchange resins, or as sparingly soluble derivatives, for example, as a sparingly soluble salt.

The compounds can also be formulated in rectal compositions such as suppositories or retention enemas, e.g., containing conventional suppository bases such as cocoa butter, polyethylene glycol, or other glycerides. The compounds can also be formulated in vaginal compositions as gels, suppositories, or as dendrimers conjugates. Compounds of the disclosure can be administered topically, that is by non-systemic administration. Formulations suitable for topical administration include liquid or semi-liquid preparations suitable for penetration through the skin such as gels, liniments, lotions, creams, ointments or pastes.

Gels for topical or transdermal administration of compounds of the disclosure can include a mixture of volatile solvents, nonvolatile solvents, and water. The volatile solvent component of the buffered solvent system can include lower (C1-C6) alkyl alcohols, lower alkyl glycols and lower glycol polymers. In certain embodiments, the volatile solvent is ethanol. The volatile solvent component is thought to act as a penetration enhancer, while also producing a cooling effect on the skin as it evaporates. The nonvolatile solvent portion of the buffered solvent system is selected from lower alkylene glycols and lower glycol polymers. In certain embodiments, propylene glycol is used. The nonvolatile solvent slows the evaporation of the volatile solvent and reduces the vapor pressure of the buffered solvent system. The amount of this nonvolatile solvent component, as with the volatile solvent, is determined by the pharmaceutical compound or drug being used. When too little of the nonvolatile solvent is in the system, the pharmaceutical compound can crystallize due to evaporation of volatile solvent, while an excess will result in a lack of bioavailability due to poor release of drug from solvent mixture. The buffer component of the buffered solvent system can be selected from any buffer commonly used in the art; in certain embodiments, water is used. There are several optional ingredients which can be added to the topical composition. These include, but are not limited to, chelators and gelling agents. Appropriate gelling agents can include, but are not limited to, semisynthetic cellulose derivatives (such as hydroxypropylmethylcellulose) and synthetic polymers, and cosmetic agents.

Lotions or liniments for application to the skin can also include an agent to hasten drying and to cool the skin, such as an alcohol or acetone, and/or a moisturizer such as glycerol or an oil such as castor oil or arachis oil.

Creams, ointments or pastes according to the disclosure are semi-solid formulations of the active ingredient for external application. They can be made by mixing the active ingredient in finely-divided or powdered form, alone or in solution or suspension in an aqueous or non-aqueous fluid, with the aid of suitable machinery, with a greasy or non-greasy base. The base can comprise hydrocarbons such as hard, soft or liquid paraffin, glycerol, beeswax, a metallic soap; a mucilage; an oil of natural origin such as almond, com, arachis, castor or olive oil; wool fat or its derivatives or a fatty acid such as steric or oleic acid together with an alcohol such as propylene glycol or a macrogel. The formulation can incorporate any suitable surface active agent such as an anionic, cationic or non-ionic surfactant such as a sorbitan ester or a polyoxyethylene derivative thereof. Suspending agents such as natural gums, cellulose derivatives or inorganic materials such as silicaceous silicas, and other ingredients such as lanolin, can also be included.

### EXEMPLIFICATION

This disclosure is further illustrated by the following examples, which should not be construed as limiting.

### Example 1

### General Materials and Methods

Compounds: All compounds were synthesized as described previously (LaLonde, J. M., et al. J. Med. Chem. 2012, 55, 4382-4396; LaLonde, J. M., et al. Med. (Chem. Lett. 2013, 4, 338-343; and LaLonde, J. M., et al. Bioorg. Med. Chem. 2011, 19, 91). The compounds were analyzed, dissolved in dimethyl sulfoxide at a stock concentration of 10-20 mM, aliquoted, and stored at −20° C. Each compound was then diluted to 1 mM in serum-free Dulbecco's modified Eagle medium (DMEM) and used for different assays.

Cell lines: 293T human embryonic kidney and Cf2Th canine thymocytes (ATCC) were grown at 37° C. and 5% CO₂ in Dulbecco's modified Eagle's medium (Invitrogen) containing 10% fetal bovine serum (Sigmna) and 100 μg/mL penicillin-streptomycin (Mediatech, Inc.). Cf2Th cells stably expressing human CCR5 and CD4 were grown in medium supplemented with 0.4 mg/mL G418 and 0.2 mg/mL hygromycin (Invitrogen).

Recombinant luciferase viruses: 293T human embryonic kidney cells were co-transfected with plasmids expressing the pCMVΔP1Δenv HIV-1 Gag-Pol packaging construct, the R5 YU2 envelope glycoproteins, or the envelope glycoprotein of the control amphotropic murine leukemia virus (A-MLV), and the firefly luciferase-expressing vector at a DNA ratio of 1:1:3 μg using the Effectene transfection reagent (Qiagen). Co-transfection produced single-round, replication-defective viruses. The virus-containing supernatants were harvested 36-40 h after transfection, spun, aliquoted, and frozen at −80° C. until further use. The reverse transcriptase (RT) activities of all viruses were measured as described in Rho, H. M., et al. Virology 1981, 112, 355-360.

Infection by single-round luciferase viruses. Cf2Th-CCR5-CD4 target cells were seeded at a density of 6×10³ cells/well in 96-well luminometer-compatible tissue culture plates (PerkinElmer) 24 h before infection. On the day of infection, NBD-556 and (+)-DMJ-II-121 (0-100 μM) were incubated with recombinant viruses (10,000 RT units) at 37° C. for 30 min. In the case of sensitization assays, a constant concentration of compounds was incubated with virus for 30 min at 37° C.; then, 17b or other antibodies (0-100 μg/mL) were added to the virus/compound mixture and incubated for an additional 30 min at 37° C. The mixtures were then added to the target cells and incubated for 48 h at 37° C.; after this time, the medium was removed from each well, and the cells were lysed by the addition of 30 μL passive lysis buffer (Promega) and three freeze-thaw cycles. An EG&G Berthold Microplate Luminometer LB 96V was used to measure the luciferase activity of each well after the addition of 100 μL of luciferin buffer (15 mM MgSO₄, 15 mM KPO₄, pH 7.8, 1 mM ATP, and 1 mM dithiothreitol) and 50 μL of 1 mM Firefly D-Luciferin Free Acid 99% (Prolume).

Stable core gp120 design. Based upon available structures of the HIV-1 gp120 core containing the entire V3 region, the original gp120 core was redesigned with additional internal changes to stabilize the coreceptor-binding region. To reduce conformational flexibility and lock the gp120 core into the receptor-bound state, two tactics were used: filling hydrophobic pockets of the core and adding inter-domain disulfide pairs. For the first tactic, cavity-filling or “F changes” (T257S and S375W) were designed to fill the Phe 43 cavity; along with other gp120 cavity-filling substitutions (M95W and A433M) were also included in the stabilized gp120 cores. Four inter-domain cysteine pairs (disulfides or DS or “CC” mutations) were introduced to lock the core into the CD4-bound, coreceptor-binding conformation. These cysteine substitutions specifically involve residues 96-275 (DS1; 1^{st} CC), 109-428 (DS2; 2^{nd} CC), 123-431 (DS3, 3^{rd} CC) and 231-267 (DS4, 4^{th} CC). The 2CC gp120 core contains DS1 and DS2; the 3CC gp120 core contains DS1, DS2 and DS3; the 4CC gp120 core contains all four internal cysteine pairs.

To enhance protein folding and expression, the V1/V2 stem was trimmed and residues were added back the V3 base beta strands to result in the new V3S unmodified core and the corresponding stable cores 2CC, 3CC and 4CC. To focus the immune response onto the conserved coreceptor-binding site, the immunodominant V1/V2 and V3 hypervariable regions were removed as described below. Previously, loop truncations demonstrated that such removal was possible; however, structural analysis suggested more optimal designs were feasible. The structure of the gp120 core with intact V3 loop showed that the previously published Gly-Ala-Gly substitution of V3 residues 298-329 (to accomplish deletion of V3) removed four hydrogen bonds from β-strand 12 and five hydrogen bonds from β-strand 13. A new substitution (V3S) that retained these hydrogen bonds and added a longer linker was modeled. Further structural analysis indicated that additional trimming of the flexible V1/V2 region to eliminate a naturally occurring cysteine pair might facilitate accommodation of additional pairs of stabilizing cysteines elsewhere in the molecule. Accordingly, a more minimal loop (V1/V2b) was modeled with a type II turn connecting strands β2 and β3, replacing nine residues (CVGAGSCNT) with an Ala-Gly-Ala tripeptide.

Protein expression, purification and characterization. The stable gp120 cores were expressed by transient transfection of the pcDNA 3.1(−) expression vector into suspension HEK293T cells in serum-free media (Life Technologies). The stable gp120 cores were purified by 17b affinity columns to a high level of homogeneity. The folding of the purified stable gp120 cores was assessed by ELISA, SPR and ITC with the conformational ligands sCD4, 17b and b12. Protein purity and molecular mass were determined by SDS-PAGE analysis followed by Coomassie Blue staining, as well as Blue Native gel electrophoresis and size-exclusion chromatography.

Animal inoculations and analysis of antisera. Approximately 12-week-old female New Zealand White rabbits were housed at the AAALAC-accredited facilities at Bioqual (Rockville, Md.) under specific pathogen-free conditions. At 4-week intervals, rabbits were inoculated intramuscularly with 50 mg of affinity-purified protein formulated in GlaxoSmithKline Adjuvant System AS01B by splitting the protein-adjuvant mix in the two hind legs. Serum was prepared, heat inactivated, and assessed for anti-gp120 ELISA titers and the presence neutralizing antibodies.

Isothermal Titration Calorimetry. Thermodynamic parameters for the binding of the different inhibitors to gp120 were obtained by isothermal titration calorimetry (ITC) using a VP-ITC microcalorimeter from MicroCal/GE Healthcare (Northampton, Mass., USA). The titrations were performed at 25° C. by injecting 10 μL aliquots of inhibitor solution into the calorimetric cell (volume ˜1.4 mL) containing gp120 at a concentration of 2 μM. The inhibitor concentration in the syringe was 40-60 μM except for NBD-556, which was prepared at a concentration of 125 μM. In all titration experiments, gp120 and the different inhibitors were equilibrated with PBS, pH 7.4, with 2% DMSO. The heat evolved upon each injection of inhibitor was obtained by integration of the calorimetric signal. The heat associated with inhibitor binding to gp120 was obtained by subtracting the heat of dilution from the heat of reaction. The enthalpy change (ΔH) and association constant (Kₐ=1/Kj) were obtained by nonlinear regression of the data.

Cold Inactivation. Recombinant virus (10,000 RT units) was incubated with either a fixed concentration of the NBD-556 analogues (50 μM for viruses with JR-FL and A-MLV Envs and 20 μM for viruses with YU2 Env) or dimethyl sulfoxide (DMSO) on ice at 4° C. for the following timepoints: 0, 2, 4, 8, 24 and 48 h. Cf2Th-CCR5-CD4 target cells were seeded at a density of 6×10 cells/well in a 96-well luminometer-compatible tissue-culture plate (PerkinElmer) 24 h before infection. The mixtures were then added to target cells and incubated for 48 h at 37° C.; after this time, the medium was removed from each well, and the cells were lysed by the addition of 30 μL passive lysis buffer (Promega) and three freeze-thaw cycles. An EG&G Berthold Microplate Luminometer LB 96V was used to measure the luciferase activity of each well after the addition of 100 μL of luciferin buffer (15 mM MgSO₄, 15 mM KPO₄, pH 7.8, 1 mM ATP, and 1 mM dithiothreitol) and 50 μL of 1 mM Firefly D-Luciferin Free Acid 99% (Prolume).

### Example 2

### Characterization of Small-Molecule CD4-Mimetic Compounds

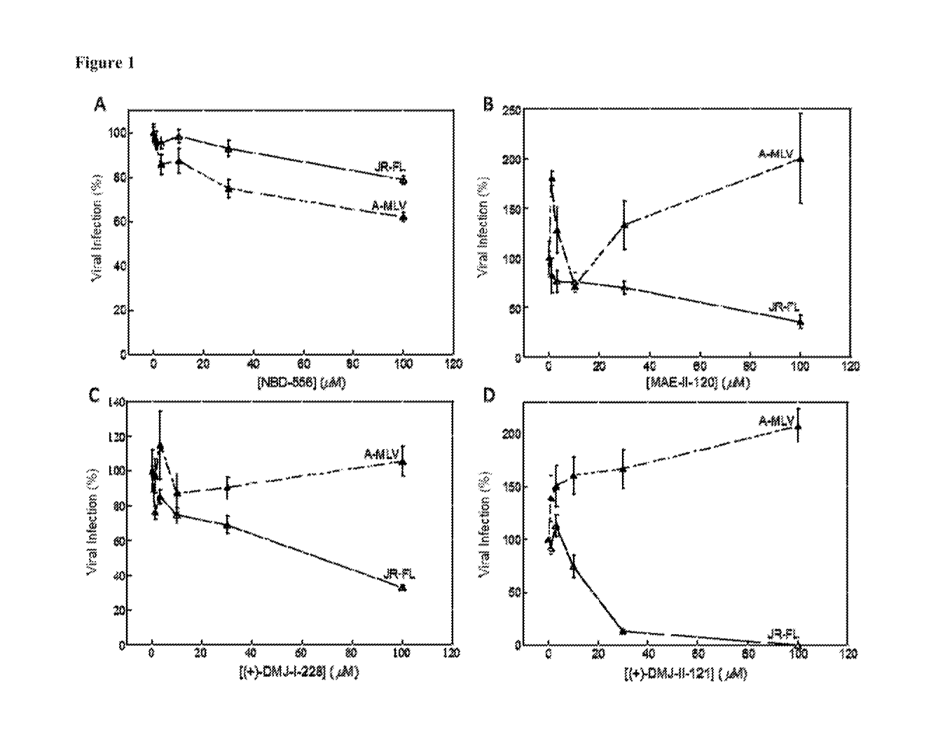

The ability of selected CD4-mimetic compounds to inhibit HIV-1 entry was examined. (+)-DMJ-I-228 and (+)-DMJ-II-121 were compared to the parental NBD-556 and an earlier analogue, (±)-MAE-II-120. Recombinant HIV-1 expressing the firefly luciferase gene was pseudotyped with different envelope glycoproteins, either HIV-1 JR-FL Env or, as a control, the amphotropic murine leukemia virus (A-MLV) envelope glycoproteins. The recombinant viruses were incubated with cells expressing CD4 and CCR5 in the presence of different concentrations of the compounds. (+)-DMJ-I-228 and (+)-DMJ-II-121 each specifically inhibited the HIV-1 JR-FL virus with an improved potency relative to the parental NBD-556 compound (FIG. 1 and FIG. 4).

The thermodynamics of the binding of each compound to the wild-type HIV-1 YU2 gp120 glycoprotein was analyzed by isothermal titration calorimetry. Relative to NBD-556, (+)-DMJ-I-228 and (+)-DMJ-II-121 each exhibited significant improvement in the ability to bind monomeric gp120 (FIG. 4). While not wishing to be bound by theory, it is believed that this increase in gp120 binding affinity is due to the fact that the entropic contribution to the binding affinity is less unfavorable than for NBD-556 (FIG. 4). Binding of CD4 and NBD-556 to gp120 is associated with an unusually large enthalpy change (ΔH) that is balanced by a large unfavorable entropic contribution (−TΔS) to the Gibbs free energy of binding. This thermodynamic signature results from large-scale conformational structuring and fixation of gp120 by CD4 and NBD-556. Relative to the binding of NBD-556, the binding of (+)-DMJ-I-228 and (+)-DMJ-II-121 appears to introduce less order in monomeric gp120.

Previous studies suggest that an increased propensity of the HIV-1 envelope glycoproteins to sample the CD4-bound conformation is associated with increased sensitivity to inactivation following incubation in the cold. The half-life on ice of recombinant HIV-1 JR-FL incubated with different NBD-566 analogues was examined. The half-life on ice of the HIV-1 JR-FL virus incubated with DMSO was >48 hours. Incubation of the HIV-1 JR-FL virus with the parental NBD-556 compound and (±)-MAE-II-120 resulted in half-lives in the cold of 15.7 and 8.7 hours, respectively (FIG. 4). Incubation with the (+)-DMJ-I-228 and (+)-DMJ-II-121 compounds increased the sensitivity of the HIV-1 JR-FL virus to cold, with half-lives of 9.1 and 1.9 hours, respectively (FIG. 4). Similar results were obtained with the HIV-1 YU2 virus (data not shown). These observations suggest that (+)-DMJ-I-228 and (+)-DMJ-II-121 each sensitize HIV-1 to cold inactivation, and are consistent with the induction of the CD4-bound state in Env by (+)-DMJ-I-228 or (+)-DMJ-II-121.

### Example 3

### (+)-DMJ-I-228 and (+)-DMJ-II-121 Sensitize HIV-1 to Neutralization by CD4-Induced and V3-Directed Antibodies

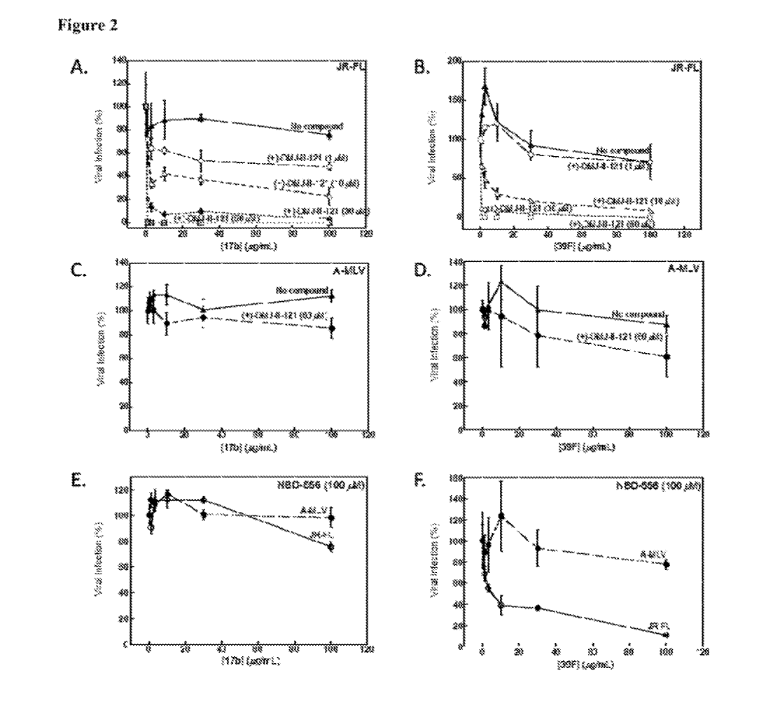

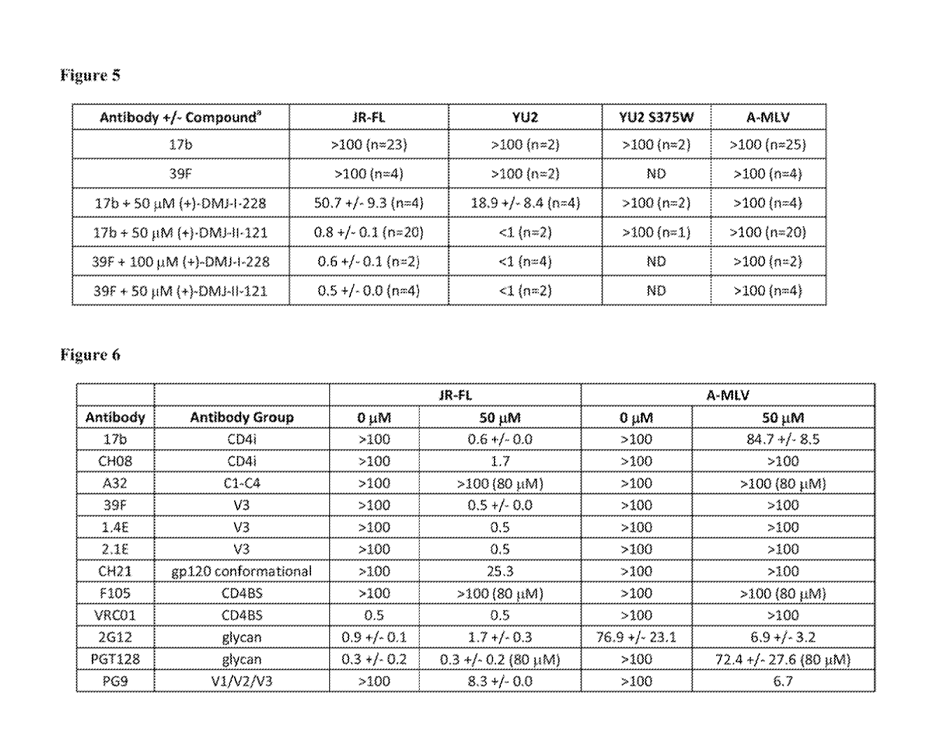

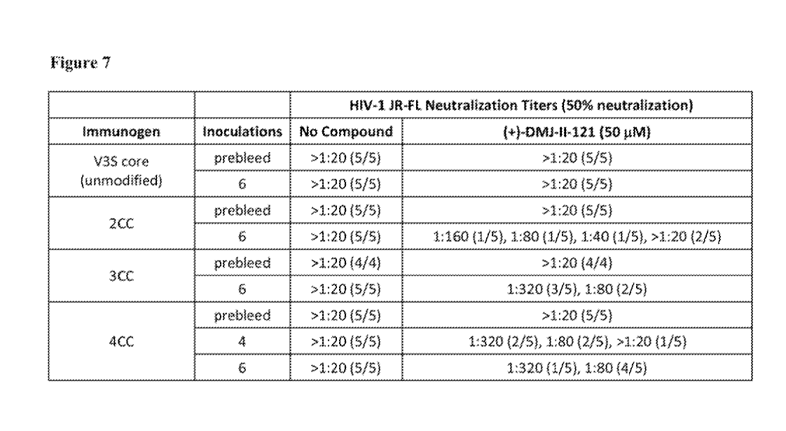

The ability of (+)-DMJ-I-228 and (+)-DMJ-II-121 to each sensitize HIV-1 to neutralization by antibodies was examined using the single-round infection of Cf2Th-CD4/CCR5 cells by recombinant HIV-1 encoding firefly luciferase. The recombinant viruses were pseudotyped with the HIV-1 Envs derived from the primary R5 HIV-1 JR-FL and YU2 isolates. Recombinant HIV-1 pseudotyped with the amphotropic murine leukemia virus (A-MLV) Env was used as a control for specificity. HIV-L neutralization was examined in the presence of sub-neutralizing concentrations of the CD4-mimetic compounds and different concentrations of monoclonal antibodies. FIG. 2 shows the results for antibodies that recognize epitopes known to be induced and/or exposed upon CD4 binding. The 17b antibody recognizes a well-conserved CD4i epitope on the HIV-1 gp120 core that is not formed or exposed in the unliganded Env of primary HIV-1 strains. The 39F antibody recognizes a conformation-dependent epitope in the gp120 V3 region of primary R5 HIV-1. In the absence of (+)-DMJ-I-228 and (+)-DMJ-II-121, neither monoclonal antibody inhibited primary HIV-1 infection at concentrations up to 100 μg/ml (FIGS. 2, A and B, and FIG. 5). (+)-DMJ-II-121 sensitized HIV-1 JR-FL to neutralization by both the 17b and 39F monoclonal antibodies (FIGS. 2, A and B). For example, in the presence of 30 μM (+)-DMJ-II-121, the 17b antibody inhibited HIV-1 JR-FL entry into cells co-expressing CD4 and CCR5 with an IC₅₀ of 0.6 μg/mL (FIG. 2A). This sensitization was dependent on the (+)-DMJ-II-121 concentration and was specific to HIV-1; there was no significant inhibition of A-MLV by either 17b or 39F antibodies in the presence of (+)-DMJ-II-121 (FIGS. 2, C and D). (+)-DMJ-I-228 and (+)-DMJ-II-121 each sensitized HIV-1 YU2 and JR-FL to neutralization by the 17b and 39F antibodies (FIG. 2, FIG. 5, and FIG. 6). Only at high concentrations did the parental compound NBD-556 weakly increase the neutralization of HIV-1 JR-FL by the 39F antibody; NBD-556 minimally affected HIV-1 JR-FL neutralization by the 17b antibody (FIGS. 2, E and F). (−)-DMJ-I-228 and (+)-DMJ-II-121 each render primary HIV-1 isolates sensitive to neutralization by monoclonal antibodies directed against CD4i and V3 epitopes on the gp120 Env.

Substitution of gp120 Ser 375 with a tryptophan residue fills the Phe 43 cavity; this substitution slightly enhances CD4 binding, but disrupts the binding of all NBD-556 analogues, including (+)-DMJ-1-228 and (+)-DMJ-II-121. None of the NBD-556 analogues tested sensitized HIV-1 JR-FL S375W to neutralization by 17b and 39F (FIG. 5). These results indicate that gp120 binding by (+)-DMJ-I-228 or (+)-DMJ-II-121 is critical for their ability to sensitize HIV-1 to neutralization by these antibodies.

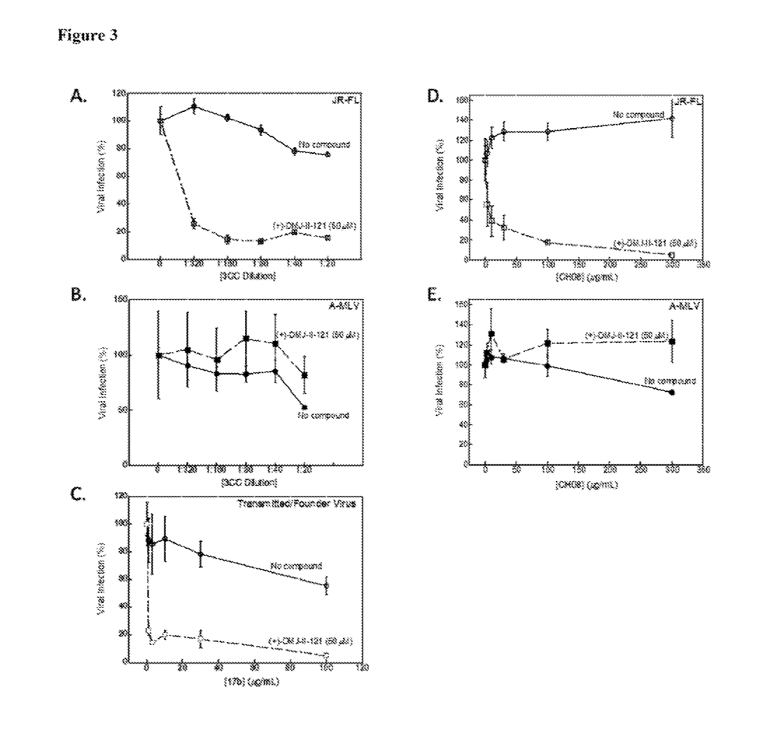

The ability of (+)-DMJ-II-121 to sensitize HIV-1 JR-FL to neutralization by a panel of anti-HIV-1 Env monoclonal antibodies (FIG. 6) was examined. The Env epitopes recognized by many of these antibodies have been characterized. The following antibodies were tested: the 17b and CH08 antibodies that recognize CD4-induced epitopes overlapping the CCR5-binding site; the A32 antibody that recognizes a discontinuous CD4-induced epitope in the C1 and C4 conserved regions of gp120 that does not overlap the CCR5-binding site; antibodies (39F, 1.4E, 2.1E) directed against the V3 region; antibodies (PGT128 and 2G12) that bind glycan-dependent gp120 outer domain epitopes: and an antibody (PG9) that recognizes quaternary structure-dependent gp120 epitopes in the V1/V2V3 region. The neutralization potency of the CD4i antibodies 17b and CH08 antibodies increased dramatically when HIV-1 was incubated with 50 μM (+)-DMJ-II-121 (FIG. 6 and FIGS. 3, D and E). In addition, (+)-DMJ-II-121 also specifically sensitized HIV-1 JR-FL to neutralization by antibodies directed against the gp120 V3 region, as well as antibody CH21, whose discontinuous gp120 epitope is not yet determined. Together, these results indicate that (+)-DMJ-I-228 and (+)-DMJ-II-121 each enhances the virus-neutralizing potency of some antibodies targeting HIV-1 Env epitopes that are induced by CD4 binding.

### Example 4

### (+)-DMJ-I-228 and (+)-DMJ-II-121 Sensitize HIV-1 to Neutralization by Antisera Elicited by an Env Immunogen

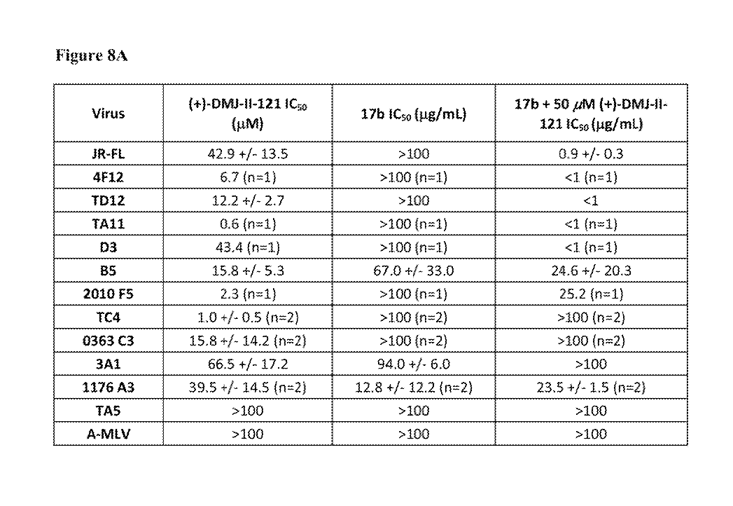

(+)-DMJ-I-228 and (+)-DMJ-II-121 were examined to determine if they could sensitize HIV-1 to neutralization by antisera elicited by an Env immunogen. Previous studies demonstrated that CD4i antibodies could be elicited in rabbits immunized with gp120 cores that were modified to stabilize the CD4-bound conformation. These stabilized gp120 cores were derived from the laboratory-adapted HIV-1 HXBc2 strain and lack the V1, V2, and V3 variable regions. X-ray crystal structures of gp120 core/two-domain CD4/XS Fab complexes were used to guide modification of the variable region deletions, substitution of cavity-filling residues, and introduction of two, three and four potential disulfide bonds (in the respective 2CC, 3CC and 4CC stabilized cores). In immunized rabbits, the stabilized gp120 cores elicited CD4i antibodies according to the following hierarchy: 4CC>3CC>2CC>a core lacking additional disulfide bonds (the V3S core). The ability of (+)-DMJ-II-121 to sensitize HIV-1 JR-FL to neutralization by immune sera from these rabbits was tested. Pre-immune sera were also tested, as negative controls. (+)-DMJ-II-121 sensitized HIV-1 JR-FL to neutralization by sera from rabbits immunized with either the 3CC or 4CC stabilized gp120 cores (FIG. 7 and FIG. 3A). Control recombinant HIV-1 pseudotyped with A-MLV Env was not neutralized by any of the sera in the presence or the absence of (+)-DMJ-H-121 (FIG. 3B). Pre-immune sera from each rabbit were used as controls and did not exhibit any neutralization (FIG. 7). Three of the five sera from rabbits immunized with the 2CC gp120 core neutralized HIV-1 JR-FL only in the presence of (+)-DMJ-II-121 (FIG. 7). None of the rabbit sera that were elicited by inoculation with the non-stabilized V3S core neutralized HIV-1 JR-FL after incubation with (+)-DMJ-II-121 (FIG. 7). Thus, neutralization of HIV-1 JR-FL after sensitization by (+)-DMJ-II-121 correlated with the previously reported levels of CD4i antibodies elicited by the different stabilized gp120 cores. The 3CC and 4CC gp120 cores better approximate the CD4-bound conformation and more efficiently elicit CD4i antibodies that can be potentiated for HIV-1 neutralization by (+)-DMJ-II-121.

### Example 5

### (+)-DMJ-I-228 and (+)-DMJ-II-121 Sensitize HIV-1 Transmitted/Founder Viruses to Neutralization by CD4-Induced 17b Antibody and Antisera Elicited by a 3CC g120 Core Immunogen

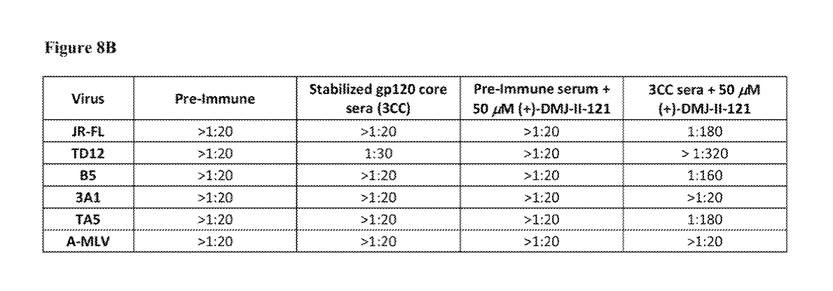

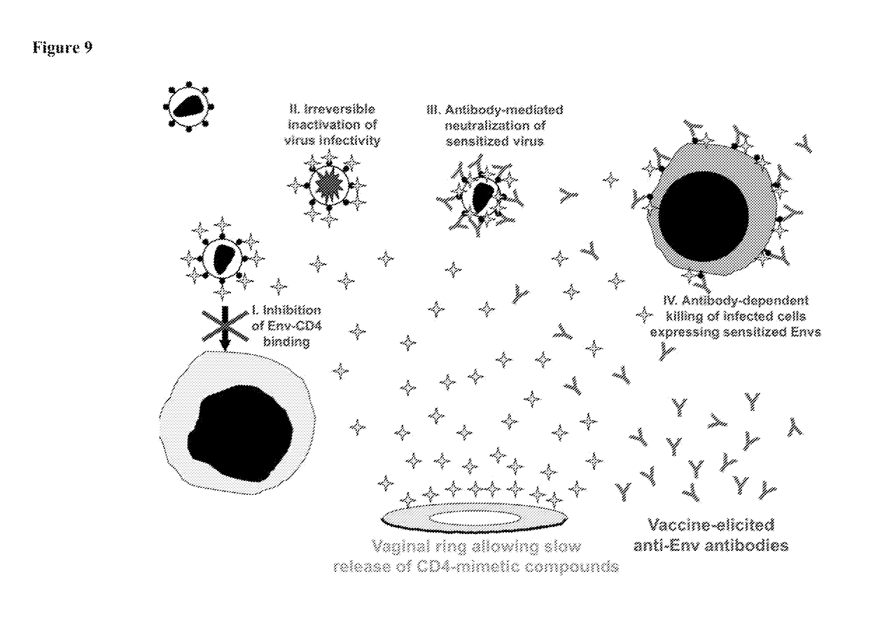

The ability of (+)-DMJ-II-121 to sensitize HIV-1 transmitted/founder viruses to neutralization by the 17b antibody (FIG. 8A) and antisera elicited by the 3CC gp120 core immunogen (FIG. 8B) was examined. In these experiments, the recombinant viruses were pseudotyped with the HIV-1 Envs derived from HIV-1 transmitted/founder viruses. Recombinant HIV-1 pseudotyped with the primary R5 HIV-1 JR-FL and the amphotropic murine leukemia virus (A-MLV) Envs served as positive and negative controls, respectively. HIV-1 neutralization was examined in the presence of sub-neutralizing concentrations of (+)-DMJ-II-121 and different concentrations of the 17b antibody or sera.

The inhibition of the transmitted/founder viruses by (+)-DMJ-II-121 or the 17b antibody alone, and the ability of (+)-DMJ-II-121 to sensitize the transmitted/founder viruses to neutralization by the 17b antibody, are reported in FIG. 8A. In the absence of (+)-DMJ-II-121, with one exception (1176 A3), the transmitted/founder viruses were negligibly inhibited by the 17b antibody. Of the ten 17b-resistant transmitted/founder HIV-1, (+)-DMJ-II-121 sensitized six viruses to neutralization by the 17b antibody. These viruses were also sensitive to inhibition by (+)-DMJ-II-121 alone. Three of the transmittcd/founder viruses tested were sensitive to (+)-DMJ-II-121 inhibition, but were not sensitized by (+)-DMJ-II-121 to 17b neutralization. One transmitted/founder virus (TA5) did not exhibit sensitivity to (+)-DMJ-II-121 alone, and was not sensitized to 17b neutralization in the presence of (+)-DMJ-II-121. Thus, a significant fraction of transmitted/founder HIV-1 are sensitive to inhibition by (+)-DMJ-II-121 and are sensitized to 17b neutralization by this compound.

Four of the transmitted/founder viruses were tested using antisera elicited by the 3CC gp120 core immunogen (FIG. 8B). Two of the transmitted/founder viruses (TD12 and B5) that were sensitive to (+)-DMJ-II-121 inhibition also exhibited increased sensitivity to neutralization by 3CC antisera in the presence of (+)-DMJ-II-121. Unexpectedly, one of the transmitted/founder viruses (TA5) that was not inhibited efficiently by (+)-DMJ-II-121 was neutralized by 3CC-elicited antisera in the presence of (+)-DMJ-II-121. Preimmune sera did not neutralize any of the transmitted/founder viruses in the presence or the absence of (+)-DMJ-II-121. There was no significant inhibition of A-MLV by either 17b or immunized sera in the presence of (+)-DMJ-II-121 (FIGS. 8, A and B). (+)-DMJ-II-121 is able to sensitize a substantial fraction of transmitted/founder HIV-1 isolates to neutralization by antiscra elicited by a 3CC gp120 core immunogen.

### Example 6

### Env-CD4 Interaction Enhances Recognition of HIV-1-Infected Cells by Sera from HIV-1-Infected Individuals

Env interaction with the CD4 receptor at the surface of infected cells is critical for efficient ADCC activity mediated by monoclonal Abs targeting CD4i Env epitopes or by sera from HIV-1-infected individuals. Env-CD4 interaction is modulated by the HIV-1 accessory proteins Nef and Vpu, which are known to modulate cell-surface levels of CD4. In addition to its role in CD4 degradation, Vpu also antagonizes a restriction factor, Tetherin/BST-2, which normally inhibits retroviral release. Viruses lacking Vpu remain trapped at the cell surface resulting in an accumulation of exposed Env. Therefore, Nef and Vpu can indirectly modulate Env-CD4 interaction at the surface of infected cells through CD4 and BST-2 downregulation. Cells infected with viruses defective for both Nef and Vpu present enhanced levels of CD4 and Env at the cell-surface, resulting in the exposure of CD4i Env epitopes recognized by ADCC-mediating Abs such as A32 and HIV-1+ sera. However, if the ability of Env to interact with CD4 is decreased by a change near the CD4-binding site (D368R), Env CD4i epitopes are poorly exposed, resulting in decreased interaction with CD4i Abs and HIV-1+ scra. Data not shown.

### Example 7

### CD4-Mimetics Sensitize HIV-1-Infected Cells to ADCC Mediated by Sera from HIV-1-Infected Individuals

The capacity of different compounds to promote the CD4-bound conformation of Env and thereby enhance Env recognition at the surface of HIV-1-infected cells by sera from HIV-1-infected individuals was explored. Soluble CD4 is the recombinant human CD4 protein lacking its transmembrane domain and is known to induce conformational changes in Env similar to those induced by CD4 expressed on target cells. Rationally designed CD4-mimetic compounds engage gp120 within the Phe43 cavity and can act as CD4 agonists, inducing thermodynamic changes in gp120 similar to those observed upon CD4 binding. Importantly, compounds of this class have been shown to sensitize HIV-1 particles to neutralization by CD4i and V3 non-neutralizing vaccine-elicited Abs.

Env present at the surface of cells infected with a wild-type (wt) virus is barely recognized by HIV-1+ sera. This is due to efficient CD4 downregulation by the virus; Env cannot engage with CD4 and therefore remains in its unbound conformation, preventing CD4i epitope exposure. CD4-mimetic compounds (CD4mc) and sCD4 promote the exposure of Env CD4i epitopes, resulting in enhanced recognition of Env at the surface of HIV-1-infected cells by HIV-1+ sera. As expected, when the ability of the virus to downregulate CD4 is impaired by deleting nef (nef- or nef-vpu−). CD4mc do not enhance Env recognition by HIV-1+ sera. In the absence of Nef, CD4 accumulates at the cell surface and interacts with Env; thus, in this case, CD4 blocks access to the Phe43 cavity, effectively competing for Env interaction. Cells infected with a wt virus express little Env at the cell-surface due to the BST-2-counteracting effect of Vpu, explaining why the enhancement by CD4mc is small. Deletion of vpr results in enhanced Env expression at the cell surface; in this context, CD4mc can engage more Env at the cell surface, thus resulting in a more pronounced effect on Env recognition by HIV-1+ sera. Under these conditions, infected cells treated with CD4-mimetic compounds reach the same level of recognition as cells infected with a nef vpu⁻ virus. Data not shown. Similar results were observed with M48U1, a miniprotein CD4 mimic that also engages the gp120 Phe 43 cavity with nanomolar affinity (data not shown).

Importantly, CD4mc enhancement of Env recognition by sera from several HIV-1-infected individuals (data not shown) is translated into higher ADCC killing of infected cells by effector PBMCs (data not shown). It is worth noting that the effect of CD4mc on Env detection and sensitization to ADCC is also observed when primary CD4 T cells from healthy individuals, rather than CEM-NKr cells, are used as targets cells (data not shown).

### Example 8

### CD4-Mimetics Enhance Recognition and Killing of Cells Infected with Primary HIV-1 Strains

To ensure that sensitization of HIV-1-infected cells by CD4-mimetics was also observed when using full-length clinically relevant primary HIV-1 isolates, we infected primary CD4 T cells with two transmitted/founder (T/F) viruses as well as their 6-month counterparts. Primary viruses are known to exhibit low Env reactivity and as such have little or no intrinsic exposure of CD4i epitopes. DMJ-I-228 and M48U1 CD4-mimetics were able to significantly enhance recognition of cells infected with the four primary viruses by HIV-1+ sera (data not shown); cells infected with T/F CH58 and its 6-month counterpart exhibited a greater enhancement of recognition when compared to T/F CH77 and its related 6-month counterpart (data not shown). This is likely related to the levels of Env present at the surface of infected cells; cells infected with T/F or 6-month CH58 presented higher surface Env levels than cells infected with T/F or 6-month CH77 (data not shown).

### INCORPORATION BY REFERENCE

The contents of all references, patent applications, patents, and published patent applications, as well as the Figures, cited throughout this application are hereby incorporated by reference.

### EQUIVALENTS

Those skilled in the art will recognize, or be able to ascertain using no more than routine experimentation, many equivalents to the specific embodiments of the disclosure described herein. Such equivalents are intended to be encompassed by the following claims.

1. A method of generating a protein binding domain that specifically binds to gp120 in a specific conformational state, comprising:
a) contacting gp120 or a fragment thereof with a compound that is:
(i) a compound of Formula VII: 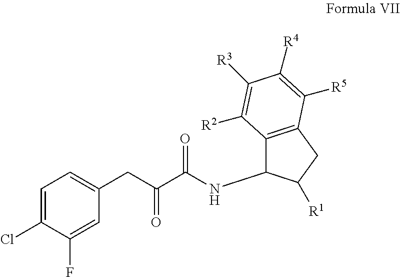
or a pharmaceutically acceptable salt or solvate thereof, wherein:
R¹ is optionally substituted amino, 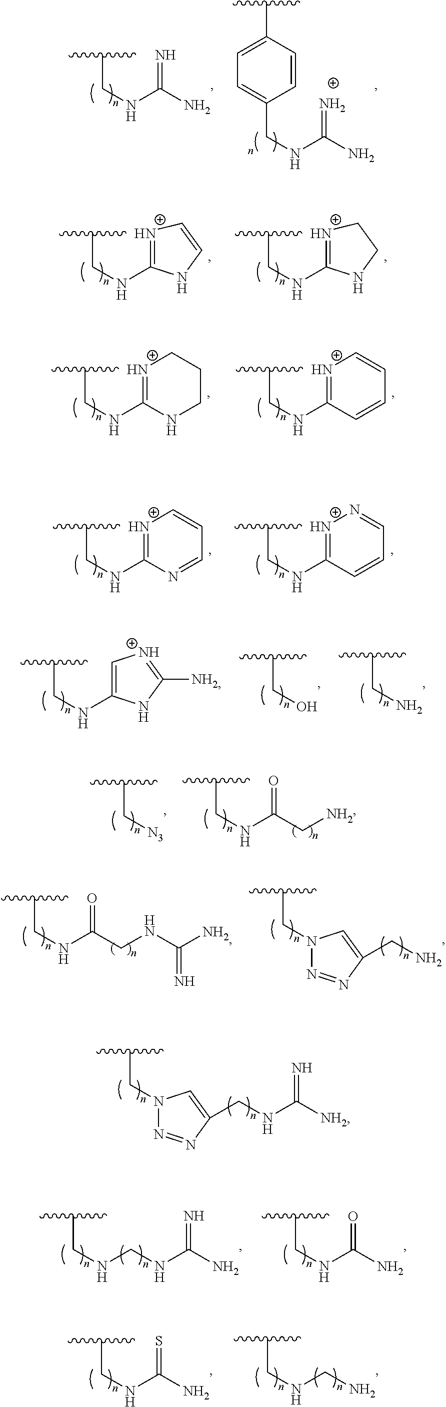 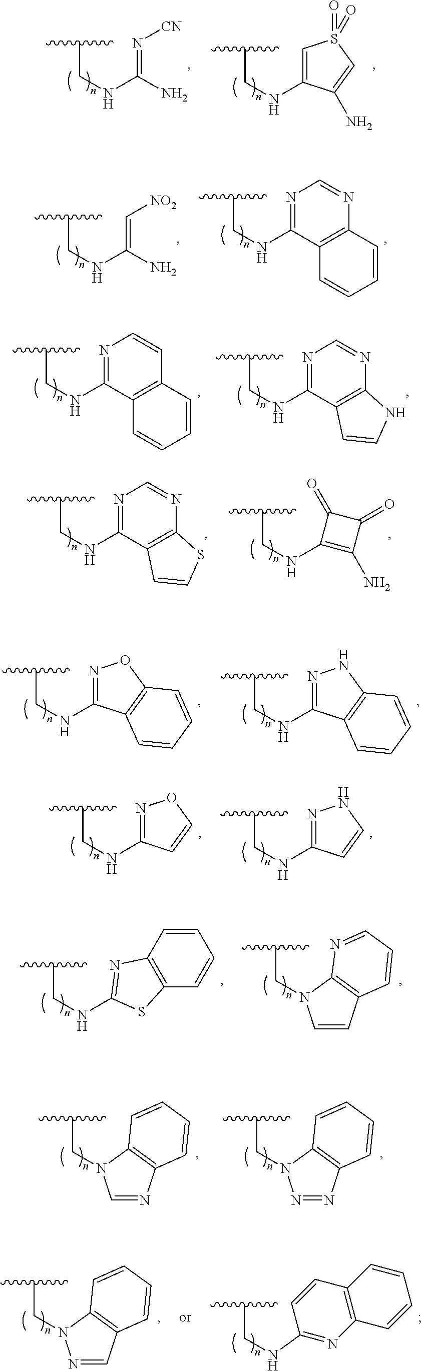
R² is H, optionally substituted alkyl, optionally substituted alkenyl, optionally substituted alkynyl, optionally substituted aryl, optionally substituted heteroaryl, hydroxy, optionally substituted alkoxy, optionally substituted amino, or halo;
R³ is H, optionally substituted alkyl, optionally substituted alkenyl, optionally substituted alkynyl, optionally substituted aryl, optionally substituted heteroaryl, hydroxy, optionally substituted alkoxy, optionally substituted amino, or halo;
R⁴ is H, optionally substituted alkyl, optionally substituted alkenyl, optionally substituted alkynyl, optionally substituted aryl, optionally substituted heteroaryl, hydroxy, optionally substituted alkoxy, optionally substituted amino, or halo;
R⁵ is H, optionally substituted alkyl, optionally substituted alkenyl, optionally substituted alkynyl, optionally substituted aryl, optionally substituted heteroaryl, hydroxy, optionally substituted alkoxy, optionally substituted amino, or halo; and
n is 0, 1, 2, 3, 4, or 5;
(ii) a compound of Formula I: 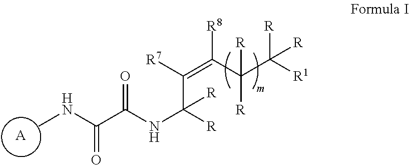
or a pharmaceutically acceptable salt or solvate thereof, wherein: 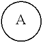
is optionally substituted aryl or heteroaryl,
R¹ is optionally substituted amino, 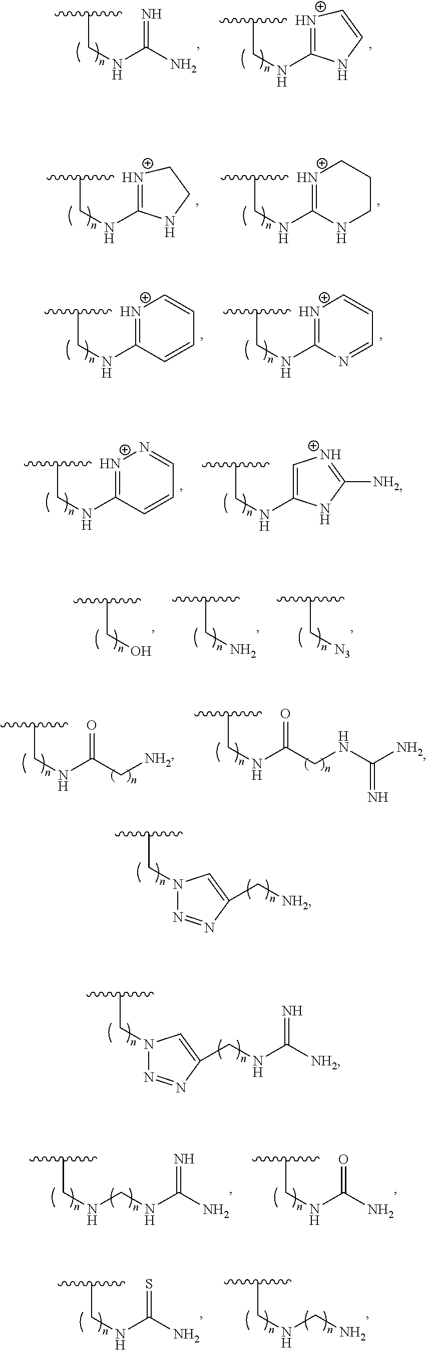 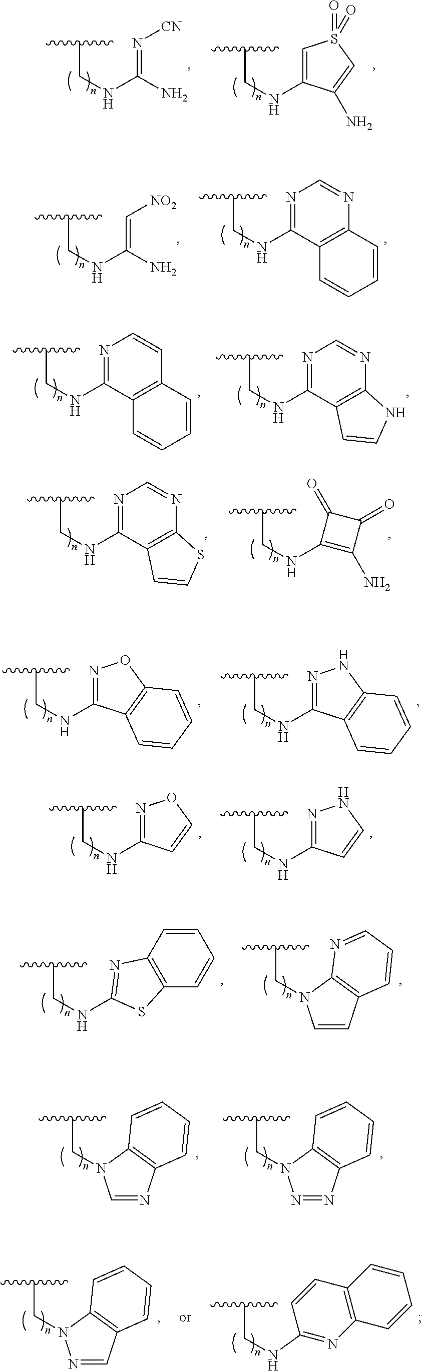
R⁷ is H, optionally substituted alkyl, optionally substituted alkenyl, optionally substituted alkynyl, optionally substituted aryl, optionally substituted heteroaryl, hydroxy, optionally substituted alkoxy, optionally substituted amino, or halo;
R⁸ is H, optionally substituted alkyl, optionally substituted alkenyl, optionally substituted alkynyl, optionally substituted aryl, optionally substituted heteroaryl, hydroxy, optionally substituted alkoxy, optionally substituted amino, or halo;
or, R⁷ and R⁸, taken together, form an optionally substituted five-membered heteroaryl ring or an optionally substituted six-membered aryl or heteroaryl ring;
m is 1, 2, 3, or 4:
each R is independently H, optionally substituted alkyl, hydroxy, optionally substituted alkoxy, optionally substituted amino, or halo; and
n is 0, 1, 2, 3, 4, or 5;
(iii) a compound of Formula II: 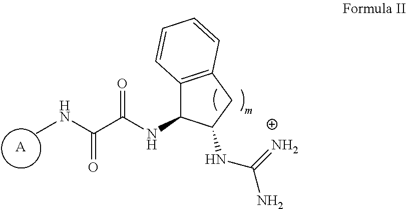
or a pharmaceutically acceptable salt or solvate thereof, wherein: 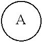
is optionally substituted aryl or optionally substituted heteroaryl; and
m is 1, 2, 3, or 4;
(iv) a compound of Formula III: 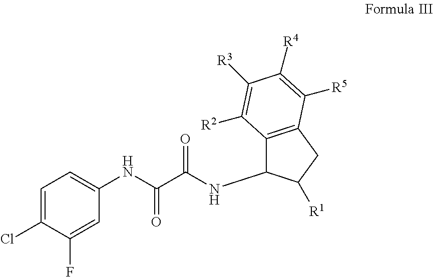
or a pharmaceutically acceptable salt or solvate thereof, wherein:
R¹ is optionally substituted amino, 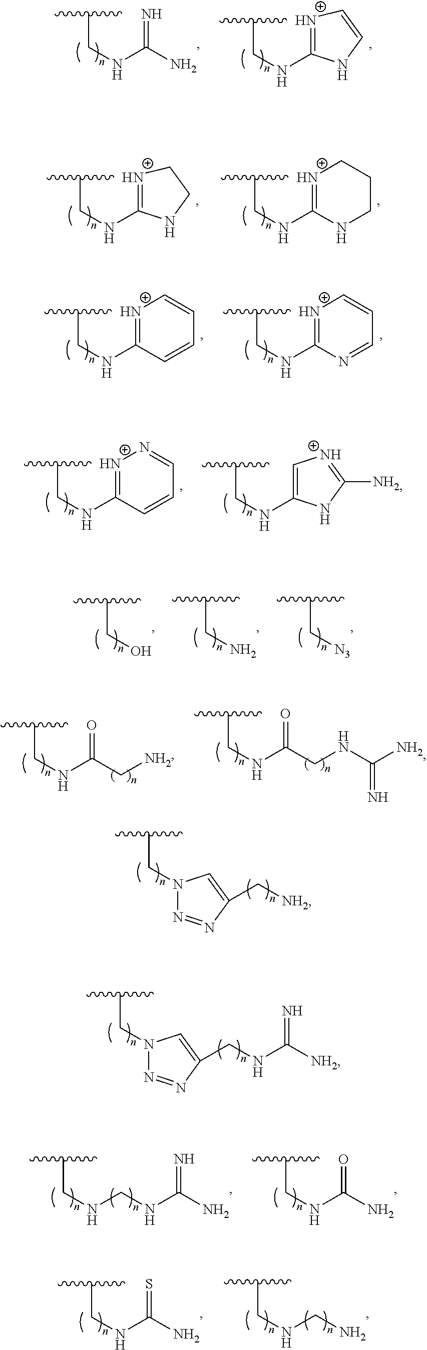 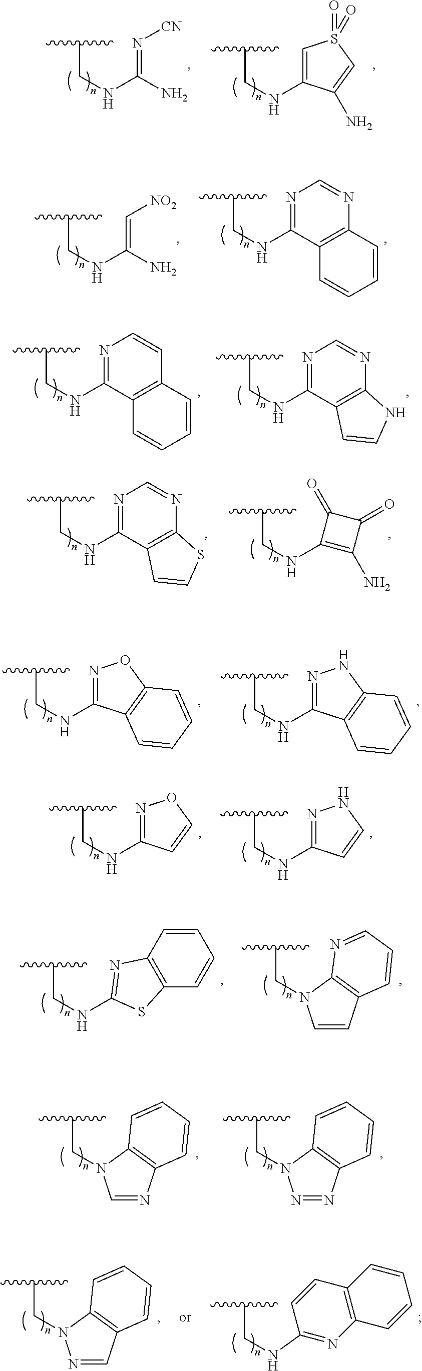
R² is H, optionally substituted alkyl, optionally substituted alkenyl, optionally substituted alkynyl, optionally substituted aryl, optionally substituted heteroaryl, hydroxy, optionally substituted alkoxy, optionally substituted amino, or halo;
R³ is H, optionally substituted alkyl, optionally substituted alkenyl, optionally substituted alkynyl, optionally substituted aryl, optionally substituted heteroaryl, hydroxy, optionally substituted alkoxy, optionally substituted amino, or halo;
R⁴ is H, optionally substituted alkyl, optionally substituted alkenyl, optionally substituted alkynyl, optionally substituted aryl, optionally substituted heteroaryl, hydroxy, optionally substituted alkoxy, optionally substituted amino, or halo;
R⁵ is H, optionally substituted alkyl, optionally substituted alkenyl, optionally substituted alkynyl, optionally substituted aryl, optionally substituted heteroaryl, hydroxy, optionally substituted alkoxy, optionally substituted amino, or halo; and
n is 0, 1, 2, 3, 4, or 5;
(v) a compound of Formula IV: 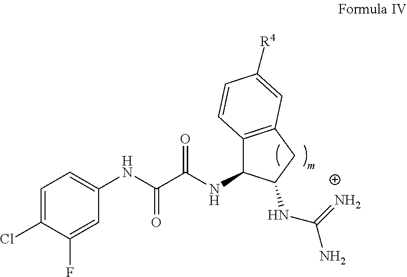
or a pharmaceutically acceptable salt or solvate thereof, wherein:
R⁴ is halo, hydroxy, thio, optionally substituted alkylsulfonamido, optionally substituted cycloalkylsulfonamido, optionally substituted amino, optionally substituted amido, optionally substituted heterocyclyl, optionally substituted heteroaryl, or optionally substituted aryl; and
m is 1, 2, 3, or 4:
(vi) a compound of Formula V: 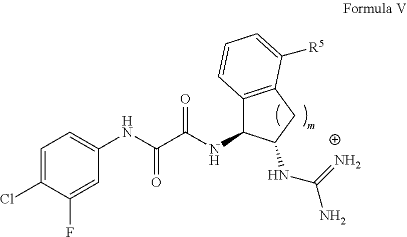
or a pharmaceutically acceptable salt or solvate thereof, wherein:
R⁵ is halo, hydroxy, thio, optionally substituted alkylsulfonamido, optionally substituted cycloalkylsulfonamido, optionally substituted amino, optionally substituted amido, optionally substituted heterocyclyl, optionally substituted heteroaryl, or optionally substituted aryl; and
m is 1, 2, 3, or 4:
(vii) a compound of Formula VI: 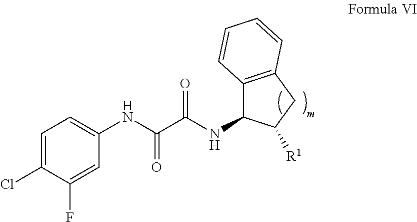
or a pharmaceutically acceptable salt or solvate thereof, wherein:
R¹ is optionally substituted amino, 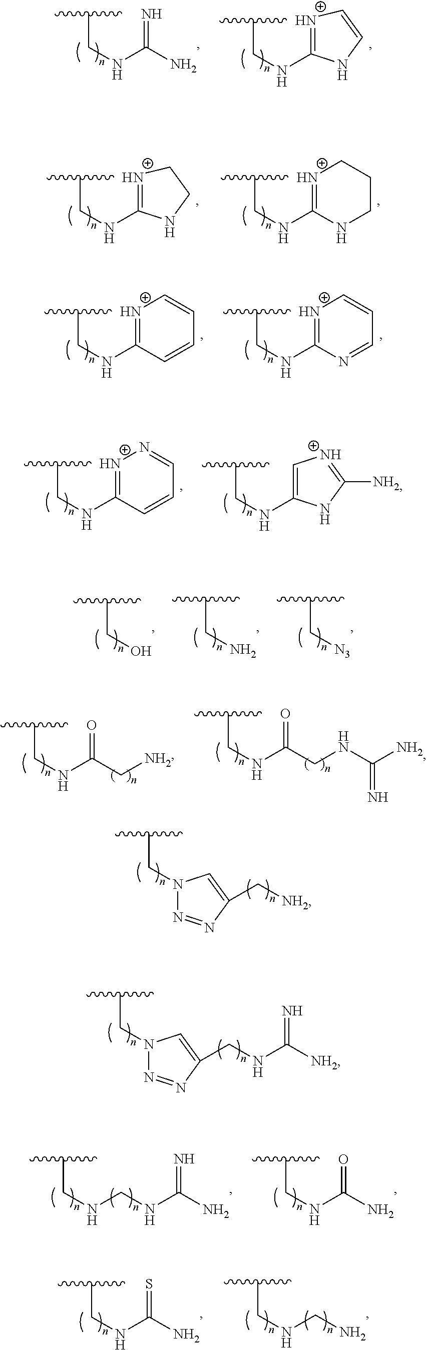 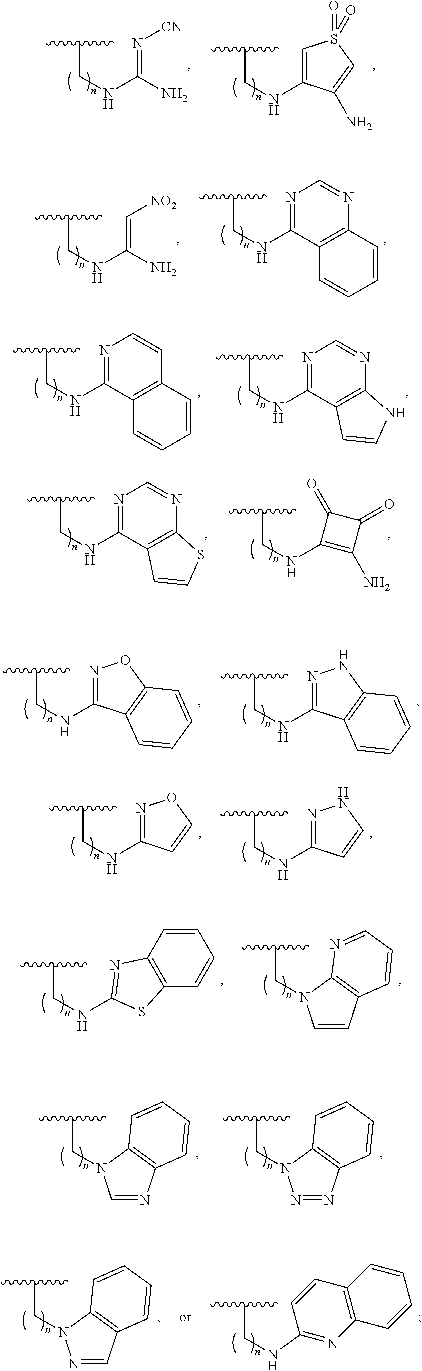
m is 1, 2, 3, or 4; and
n is 0, 1, 2, 3, 4, or 5; or
(viii) a compound of Formula VIII, 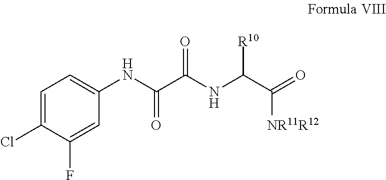
or a pharmaceutically acceptable salt or solvate thereof, wherein:
R¹⁰ is optionally substituted aryl, optionally substituted heteroaryl, optionally substituted cycloalkyl, optionally substituted heterocycloalkyl, optionally substituted alkyl, or optionally substituted alkenyl;
R¹¹ is H, optionally substituted alkyl, optionally substituted alkenyl, optionally substituted alkynyl, optionally substituted aryl, or optionally substituted heteroaryl; and
R¹² is optionally substituted alkyl, optionally substituted alkenyl, optionally substituted alkynyl, optionally substituted aryl, or optionally substituted heteroaryl;
thereby forming gp120 or the fragment thereof in the specific conformational state; and
b) generating antibodies to gp120 or the fragment thereof in the specific conformation state, wherein the compound is optionally bound to the gp120 or the fragment thereof in the specific conformational state.

2. The method of claim 1, wherein the protein binding domain is an antibody.

3. The method of claim 1, wherein the compound is a compound of Formula VII.

4. The method of claim 1, wherein the compound is

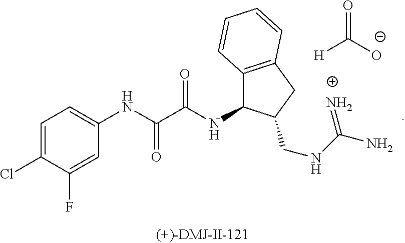

5. The method of claim 1, wherein the compound is

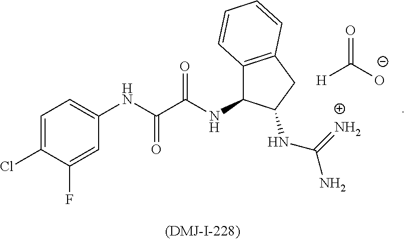

6. A method of neutralizing HIV-1 comprising:
contacting HIV-1 with an effective amount of:
(i) a compound of 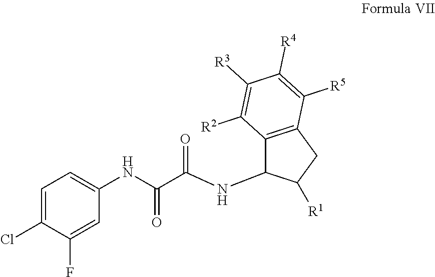
or a pharmaceutically acceptable salt or solvate thereof, wherein:
R¹ is optionally substituted amino, 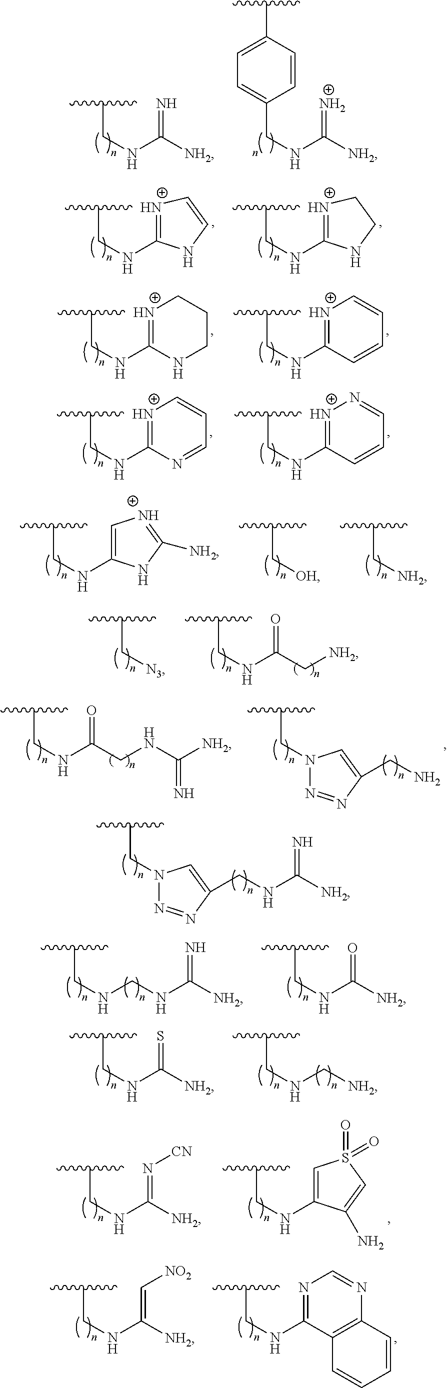 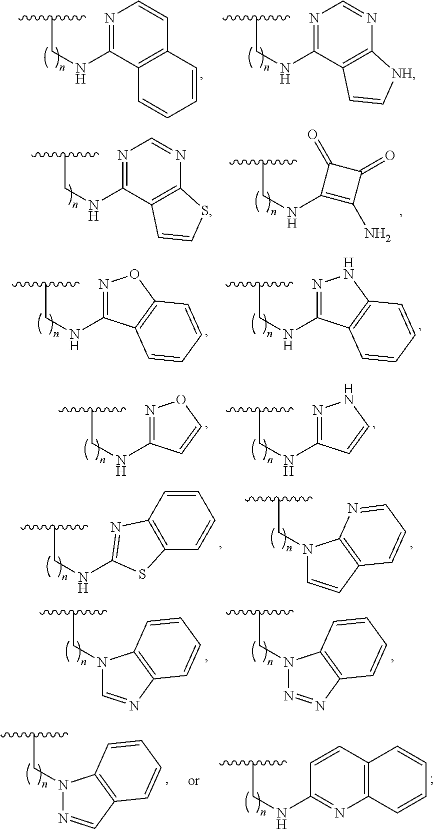
R² is H, optionally substituted alkyl, optionally substituted alkenyl, optionally substituted alkynyl, optionally substituted aryl, optionally substituted heteroaryl, hydroxy, optionally substituted alkoxy, optionally substituted amino, or halo;
R³ is H, optionally substituted alkyl, optionally substituted alkenyl, optionally substituted alkynyl, optionally substituted aryl, optionally substituted heteroaryl, hydroxy, optionally substituted alkoxy, optionally substituted amino, or halo;
R⁴ is H, optionally substituted alkyl, optionally substituted alkenyl, optionally substituted alkynyl, optionally substituted aryl, optionally substituted heteroaryl, hydroxy, optionally substituted alkoxy, optionally substituted amino, or halo;
R⁵ is H, optionally substituted alkyl, optionally substituted alkenyl, optionally substituted alkynyl, optionally substituted aryl, optionally substituted heteroaryl, hydroxy, optionally substituted alkoxy, optionally substituted amino, or halo; and
n is 0, 1, 2, 3, 4, or 5;
(ii) a compound of Formula I: 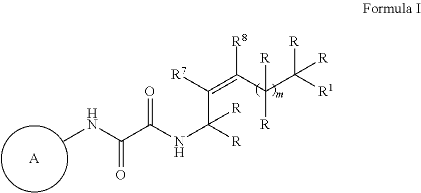
or a pharmaceutically acceptable salt or solvate thereof, wherein: 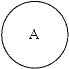
is optionally substituted aryl or heteroaryl;
R¹ is optionally substituted amino, 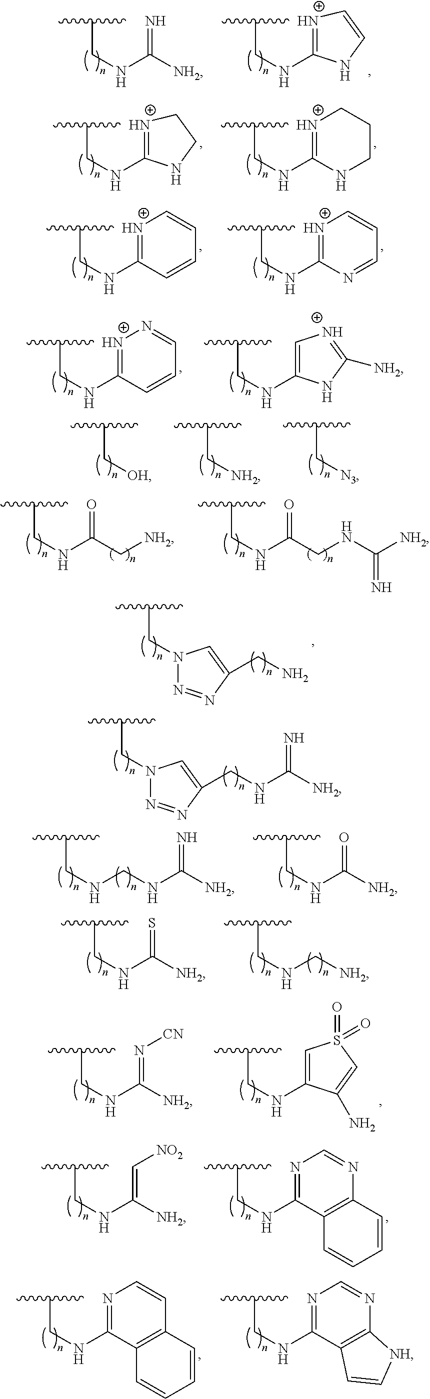 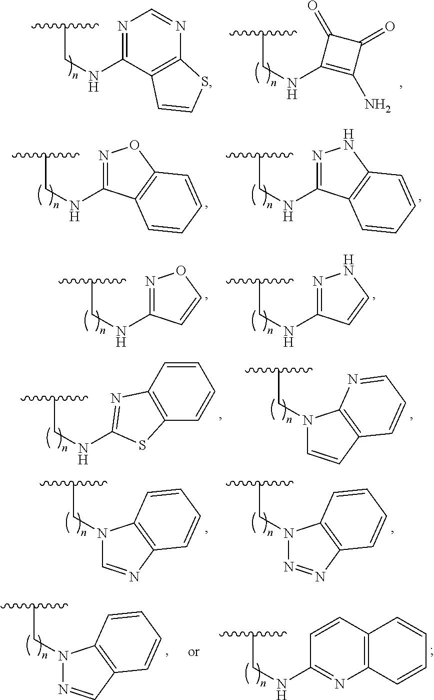
R⁷ is H, optionally substituted alkyl, optionally substituted alkenyl, optionally substituted alkynyl, optionally substituted aryl, optionally substituted heteroaryl, hydroxy, optionally substituted alkoxy, optionally substituted amino, or halo;
R⁸ is H, optionally substituted alkyl, optionally substituted alkenyl, optionally substituted alkynyl, optionally substituted aryl, optionally substituted heteroaryl, hydroxy, optionally substituted alkoxy, optionally substituted amino, or halo;
or, R⁷ and R⁸, taken together, form an optionally substituted five-membered heteroaryl ring or an optionally substituted six-membered aryl or heteroaryl ring;
m is 1, 2, 3, or 4;
each R is independently H, optionally substituted alkyl, hydroxy, optionally substituted alkoxy, optionally substituted amino, or halo; and
n is 0, 1, 2, 3, 4, or 5;
(iii) a compound of Formula II: 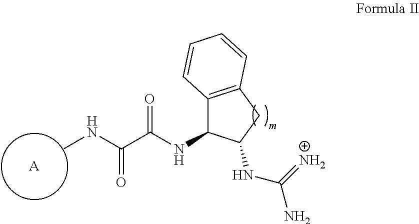
or a pharmaceutically acceptable salt or solvate thereof, wherein: 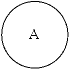
is optionally substituted aryl or optionally substituted heteroaryl; and
m is 1, 2, 3, or 4:
(iv) a compound of Formula III: 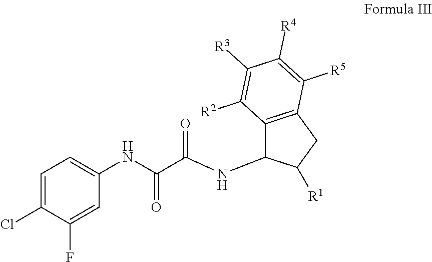
or a pharmaceutically acceptable salt or solvate thereof, wherein:
R¹ is optionally substituted amino, 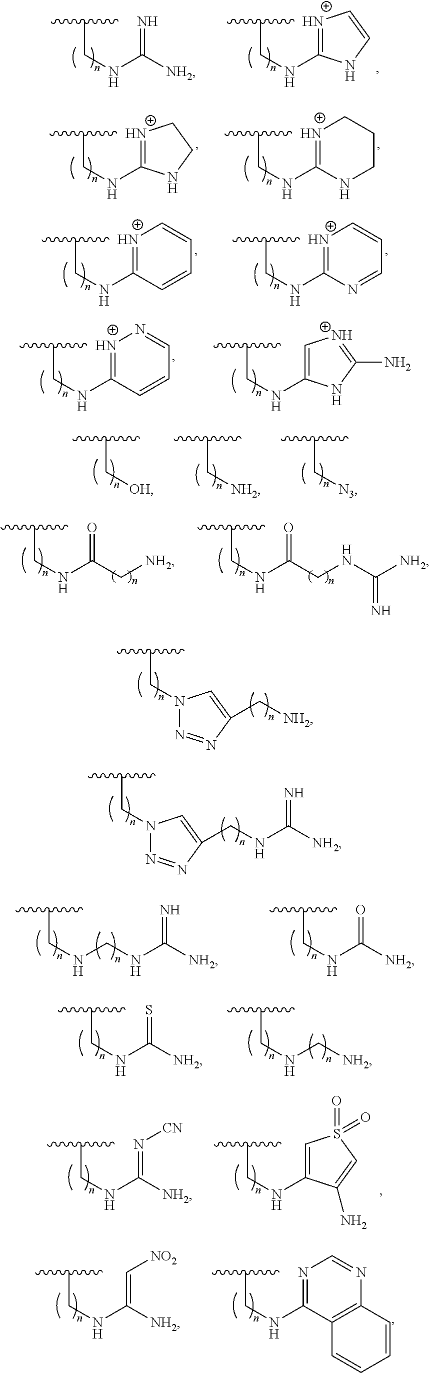 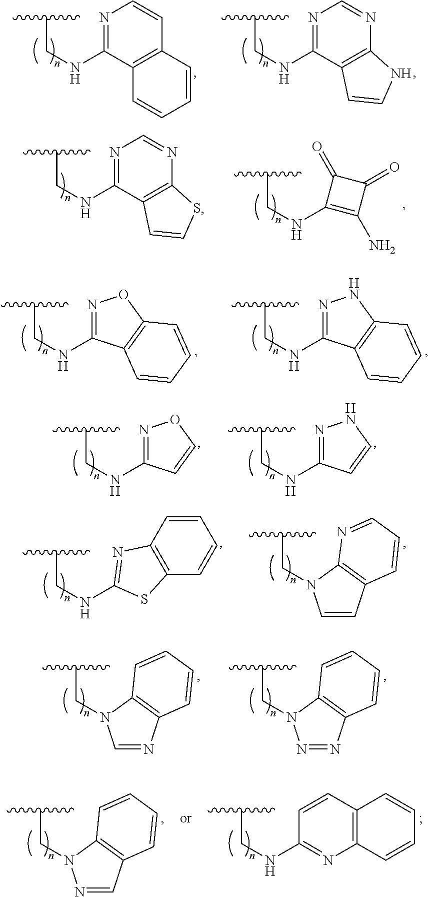
R² is H, optionally substituted alkyl, optionally substituted alkenyl, optionally substituted alkynyl, optionally substituted aryl, optionally substituted heteroaryl, hydroxy, optionally substituted alkoxy, optionally substituted amino, or halo;
R³ is H, optionally substituted alkyl, optionally substituted alkenyl, optionally substituted alkynyl, optionally substituted aryl, optionally substituted heteroaryl, hydroxy, optionally substituted alkoxy, optionally substituted amino, or halo;
R⁴ is H, optionally substituted alkyl, optionally substituted alkenyl, optionally substituted alkynyl, optionally substituted aryl, optionally substituted heteroaryl, hydroxy, optionally substituted alkoxy, optionally substituted amino, or halo;
R⁵ is H, optionally substituted alkyl, optionally substituted alkenyl, optionally substituted alkynyl, optionally substituted aryl, optionally substituted heteroaryl, hydroxy, optionally substituted alkoxy, optionally substituted amino, or halo; and
n is 0, 1, 2, 3, 4, or 5;
(v) a compound of Formula IV: 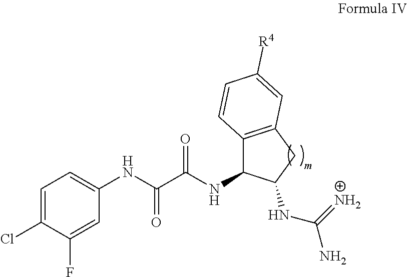
or a pharmaceutically acceptable salt or solvate thereof, wherein:
R⁴ is halo, hydroxy, thio, optionally substituted alkylsulfonamido, optionally substituted cycloalkylsulfonamido, optionally substituted amino, optionally substituted amido, optionally substituted heterocyclyl, optionally substituted heteroaryl, or optionally substituted aryl; and
m is 1, 2, 3, or 4;
(vi) a compound of Formula V: 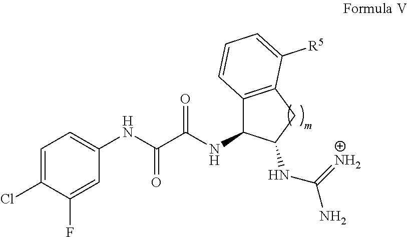
or a pharmaceutically acceptable salt or solvate thereof, wherein:
R⁵ is halo, hydroxy, thio, optionally substituted alkylsulfonamido, optionally substituted cycloalkylsulfonamido, optionally substituted amino, optionally substituted amido, optionally substituted heterocyclyl, optionally substituted heteroaryl, or optionally substituted aryl; and
m is 1, 2, 3, or 4:
(vii) a compound of Formula VI: 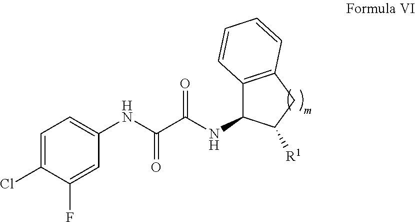
or a pharmaceutically acceptable salt or solvate thereof, wherein:
R¹ is optionally substituted amino, 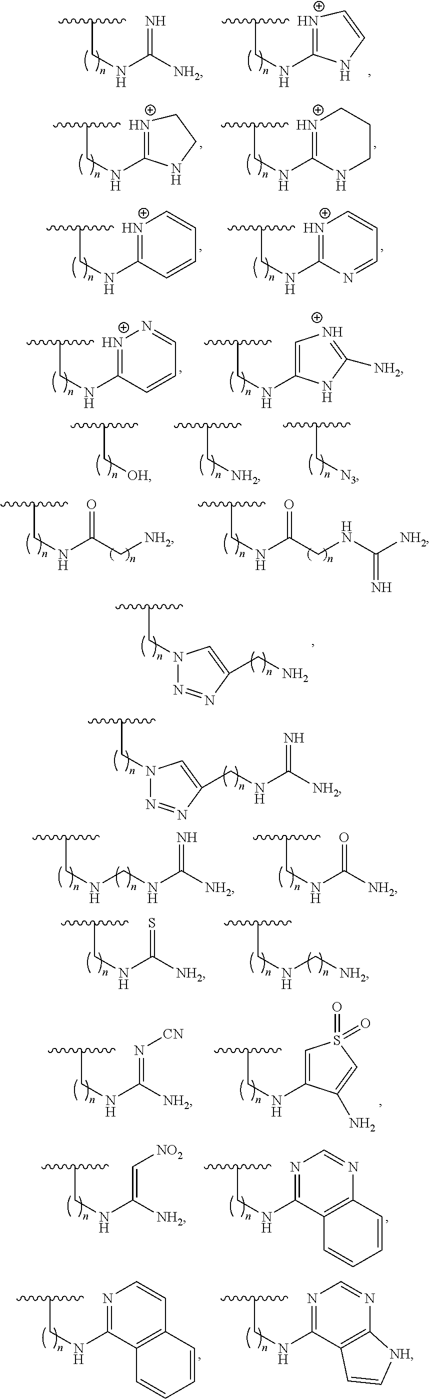 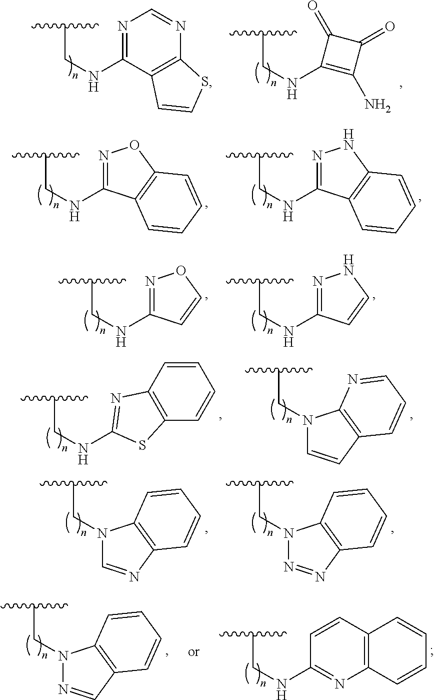
m is 1, 2, 3, or 4; and
n is 0, 1, 2, 3, 4, or 5; or
(viii) a compound of Formula VIII: 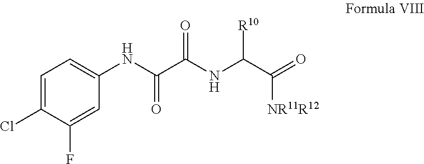
or a pharmaceutically acceptable salt or solvate thereof, wherein:
R¹⁰ is optionally substituted aryl, optionally substituted heteroaryl, optionally substituted cycloalkyl, optionally substituted heterocycloalkyl, optionally substituted alkyl, or optionally substituted alkenyl;
R¹¹ is H, optionally substituted alkyl, optionally substituted alkenyl, optionally substituted alkynyl, optionally substituted aryl, or optionally substituted heteroaryl; and
R¹² is optionally substituted alkyl, optionally substituted alkenyl, optionally substituted alkynyl, optionally substituted aryl, or optionally substituted heteroaryl;
thereby forming HIV-1 having gp120 in a specific conformational state; and
contacting the HIV-1 in the specific conformational state with an antibody, wherein the compound is optionally bound to the HIV-1 in the specific conformational state.

7. A method of treating or preventing HIV infection comprising:
administering to a subject in need thereof, a therapeutically effective amount of an antibody; and
co-administering to the subject an effective amount of:
(i) a compound of Formula VII: 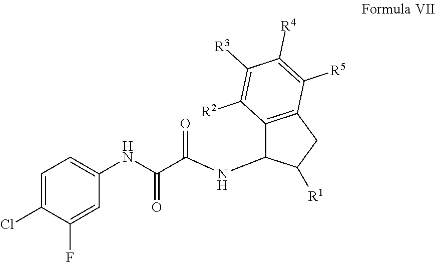
or a pharmaceutically acceptable salt or solvate thereof, wherein:
R¹ is optionally substituted amino, 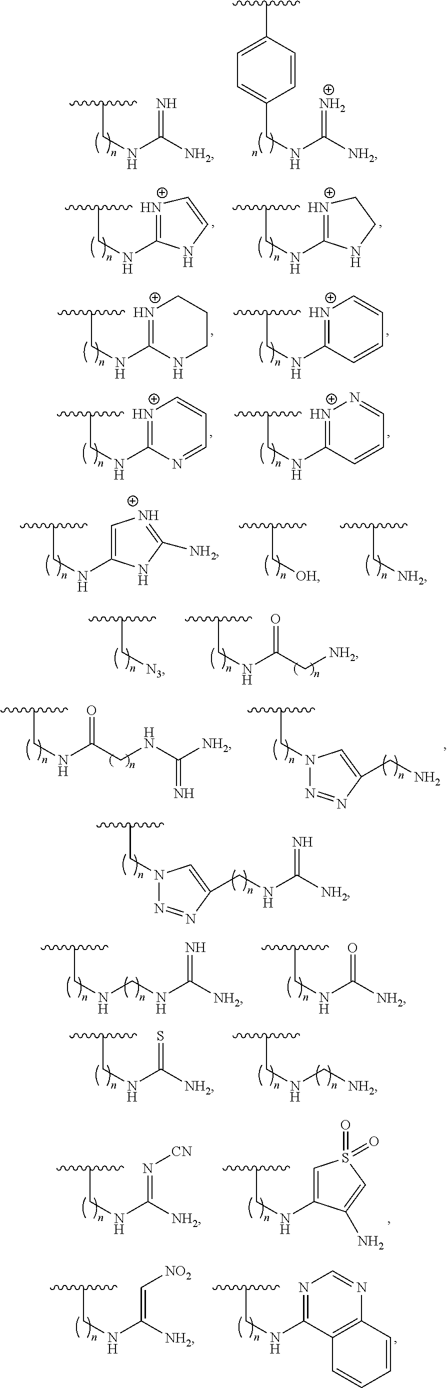 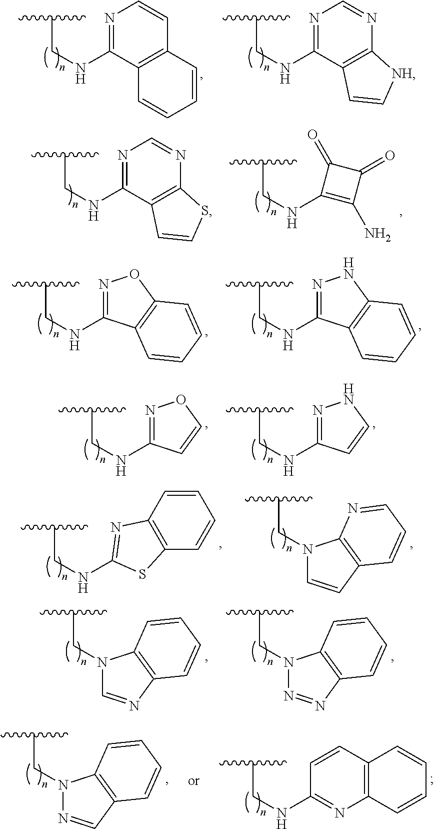
R² is H, optionally substituted alkyl, optionally substituted alkenyl, optionally substituted alkynyl, optionally substituted aryl, optionally substituted heteroaryl, hydroxy, optionally substituted alkoxy, optionally substituted amino, or halo;
R³ is H, optionally substituted alkyl, optionally substituted alkenyl, optionally substituted alkynyl, optionally substituted aryl, optionally substituted heteroaryl, hydroxy, optionally substituted alkoxy, optionally substituted amino, or halo;
R⁴ is H, optionally substituted alkyl, optionally substituted alkenyl, optionally substituted alkynyl, optionally substituted aryl, optionally substituted heteroaryl, hydroxy, optionally substituted alkoxy, optionally substituted amino, or halo;
R⁵ is H, optionally substituted alkyl, optionally substituted alkenyl, optionally substituted alkynyl, optionally substituted aryl, optionally substituted heteroaryl, hydroxy, optionally substituted alkoxy, optionally substituted amino, or halo; and
n is 0, 1, 2, 3, 4, or 5;
(ii) a compound of Formula I: 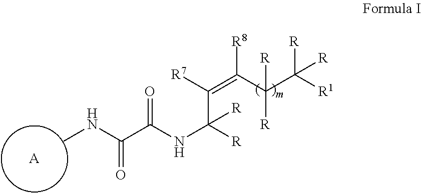
or a pharmaceutically acceptable salt or solvate thereof, wherein: 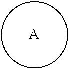
is optionally substituted aryl or heteroaryl;
R¹ is optionally substituted amino, 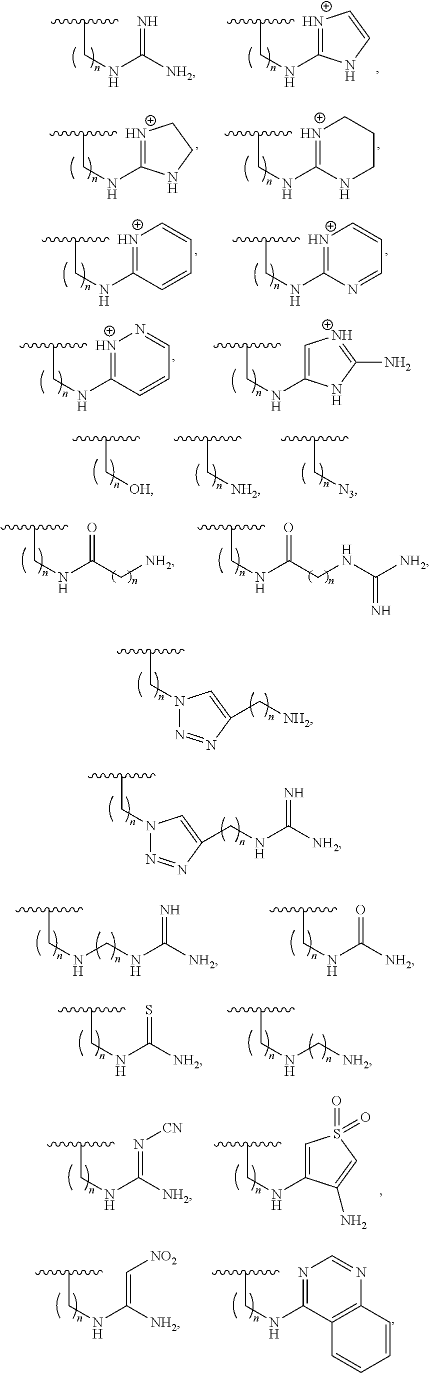 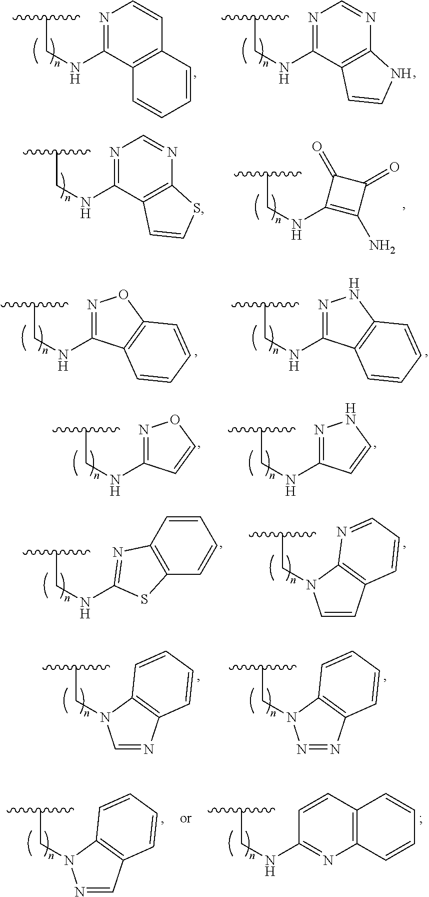
R⁷ is H, optionally substituted alkyl, optionally substituted alkenyl, optionally substituted alkynyl, optionally substituted aryl, optionally substituted heteroaryl, hydroxy, optionally substituted alkoxy, optionally substituted amino, or halo;
R⁸ is H, optionally substituted alkyl, optionally substituted alkenyl, optionally substituted alkynyl, optionally substituted aryl, optionally substituted heteroaryl, hydroxy, optionally substituted alkoxy, optionally substituted amino, or halo;
or, R⁷ and R⁸, taken together, form an optionally substituted five-membered heteroaryl ring or an optionally substituted six-membered aryl or heteroaryl ring;
m is 1, 2, 3, or 4;
each R is independently H, optionally substituted alkyl, hydroxy, optionally substituted alkoxy, optionally substituted amino, or halo; and
n is 0, 1, 2, 3, 4, or 5;
(iii) a compound of Formula II: 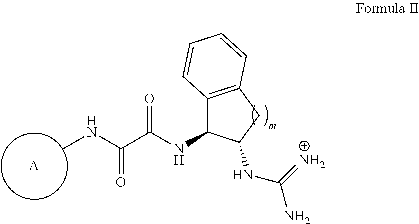
or a pharmaceutically acceptable salt or solvate thereof, wherein: 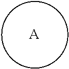
is optionally substituted aryl or optionally substituted heteroaryl; and
m is 1, 2, 3, or 4;
(iv) a compound of Formula III: 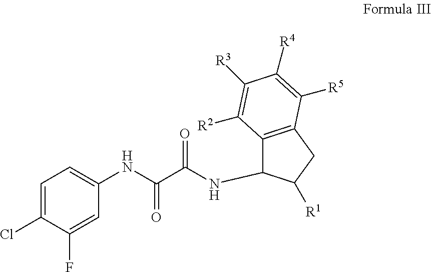
or a pharmaceutically acceptable salt or solvate thereof, wherein:
R¹ is optionally substituted amino, 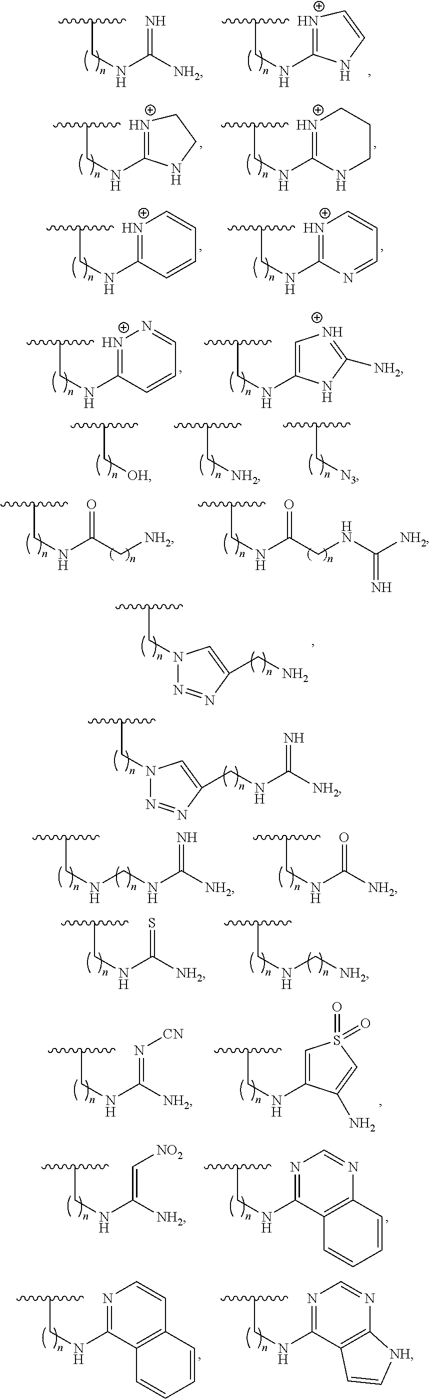 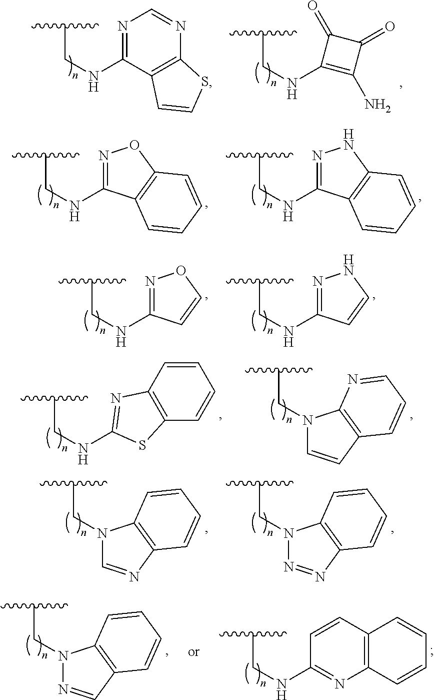
R² is H, optionally substituted alkyl, optionally substituted alkenyl, optionally substituted alkynyl, optionally substituted aryl, optionally substituted heteroaryl, hydroxy, optionally substituted alkoxy, optionally substituted amino, or halo;
R³ is H, optionally substituted alkyl, optionally substituted alkenyl, optionally substituted alkynyl, optionally substituted aryl, optionally substituted heteroaryl, hydroxy, optionally substituted alkoxy, optionally substituted amino, or halo;
R⁴ is H, optionally substituted alkyl, optionally substituted alkenyl, optionally substituted alkynyl, optionally substituted aryl, optionally substituted heteroaryl, hydroxy, optionally substituted alkoxy, optionally substituted amino, or halo;
R⁵ is H optionally substituted alkyl, optionally substituted alkenyl optionally substituted alkynyl, optionally substituted aryl, optionally substituted heteroaryl, hydroxy, optionally substituted alkoxy, optionally substituted amino, or halo; and
n is 0, 1, 2, 3, 4, or 5;
(v) a compound of Formula IV: 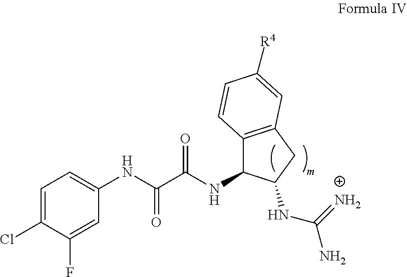
or a pharmaceutically acceptable salt or solvate thereof, wherein:
R⁴ is halo, hydroxy, thio, optionally substituted alkylsulfonamido, optionally substituted cycloalkylsulfonamido, optionally substituted amino, optionally substituted amido, optionally substituted heterocyclyl, optionally substituted heteroaryl, or optionally substituted aryl; and
m is 1, 2, 3, or 4;
(vi) a compound of Formula V: 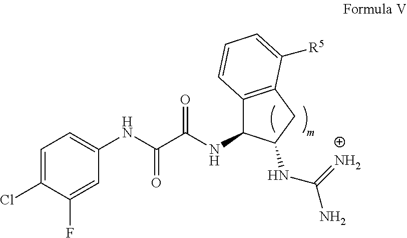
or a pharmaceutically acceptable salt or solvate thereof, wherein:
R⁵ is halo, hydroxy, thio, optionally substituted alkylsulfonamido, optionally substituted cycloalkylsulfonamido, optionally substituted amino, optionally substituted amido, optionally substituted heterocyclyl, optionally substituted heteroaryl, or optionally substituted aryl; and
m is 1, 2, 3, or 4:
(vii) a compound of Formula VI: 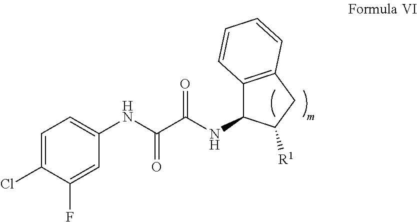
or a pharmaceutically acceptable salt or solvate thereof, wherein:
R¹ is optionally substituted amino, 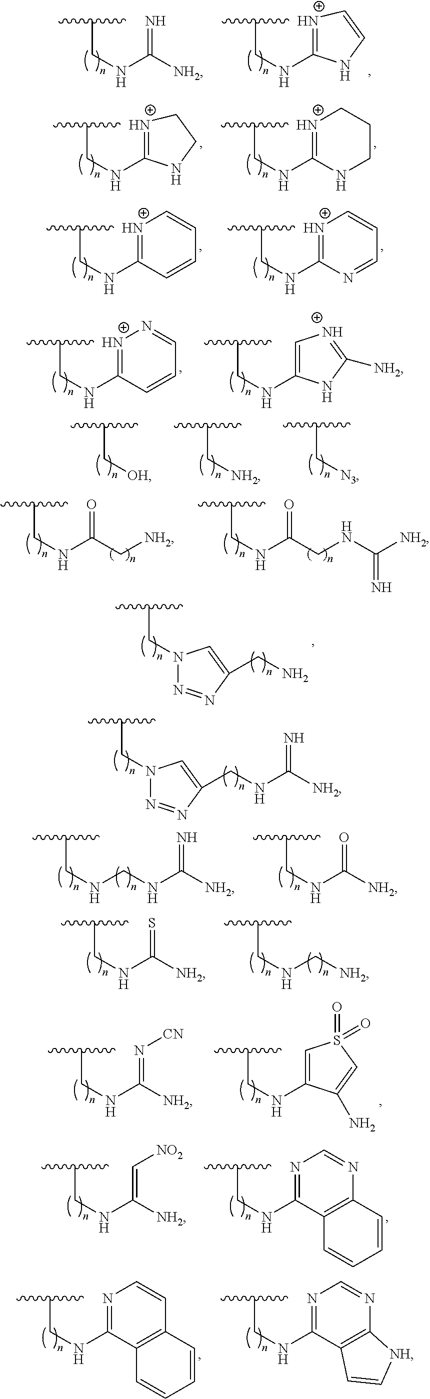 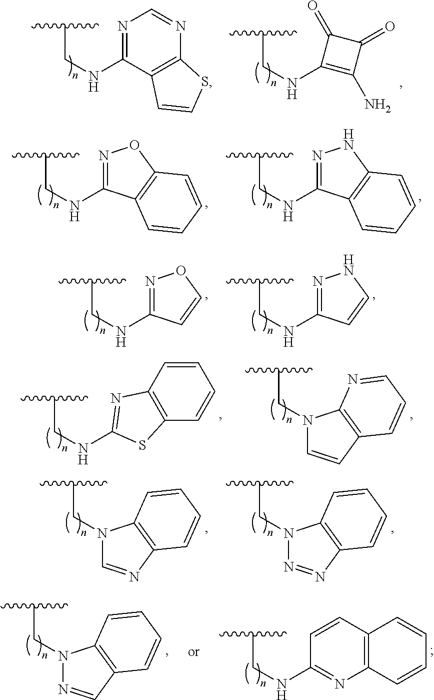
m is 1, 2, 3, or 4; and
n is 0, 1, 2, 3, 4, or 5; or
(viii) a compound of Formula VIII, 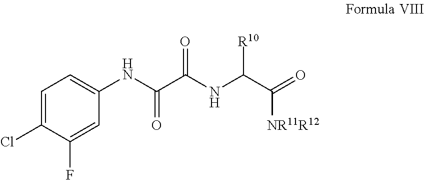
or a pharmaceutically acceptable salt or solvate thereof, wherein:
R¹⁰ is optionally substituted aryl, optionally substituted heteroaryl, optionally substituted cycloalkyl, optionally substituted heterocycloalkyl, optionally substituted alkyl, or optionally substituted alkenyl;
R¹¹ is H, optionally substituted alkyl, optionally substituted alkenyl, optionally substituted alkynyl, optionally substituted aryl, or optionally substituted heteroaryl; and
R¹² is optionally substituted alkyl, optionally substituted alkenyl, optionally substituted alkynyl, optionally substituted aryl, or optionally substituted heteroaryl.

8. The method of claim 6, wherein the antibody is a monoclonal antibody.

9. The method of claim 6, wherein the antibody is a monoclonal antibody directed against CD4-induced (CD4i) epitopes or the V3 region.

10. The method of claim 6, wherein the antibody is an anti-gp120 antibody.

11. The method of claim 6, wherein the HIV is primary HIV-1 JR-FL or a transmitted/founder virus.

12. A complex comprising (i) a compound of Formula VII, Formula I, Formula II, Formula III, Formula IV, Formula V, Formula VI, or Formula VIII, (ii) gp120 in a functional conformational state, and (iii) optionally, an antibody; wherein:
(a) said compound of Formula VII is: 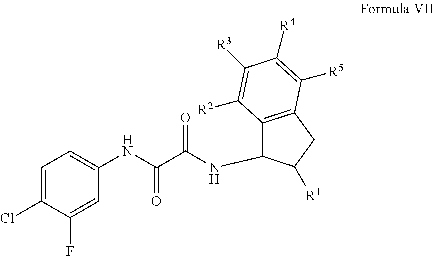
or a pharmaceutically acceptable salt or solvate thereof:
R¹ is optionally substituted amino, 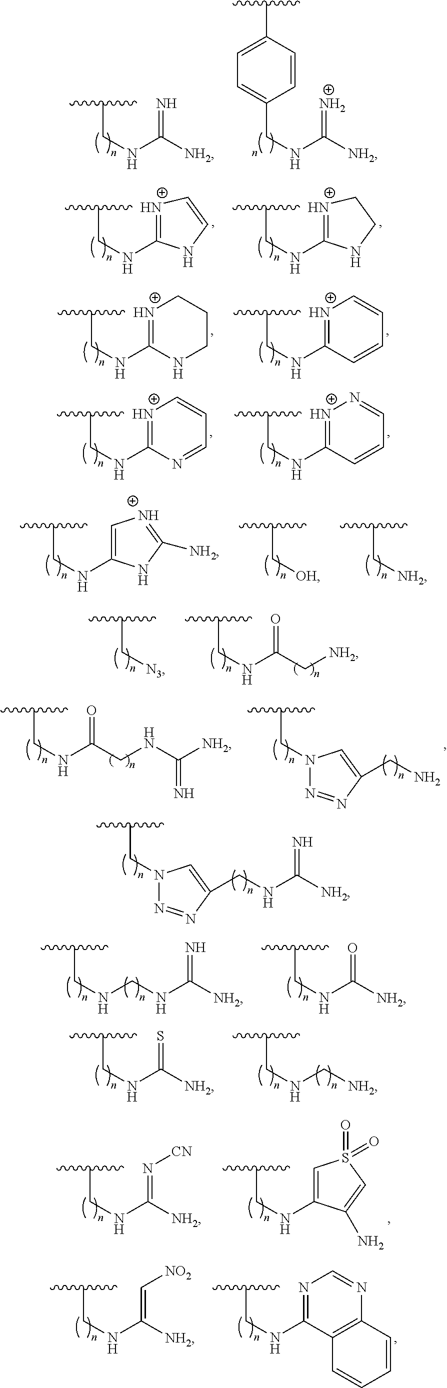 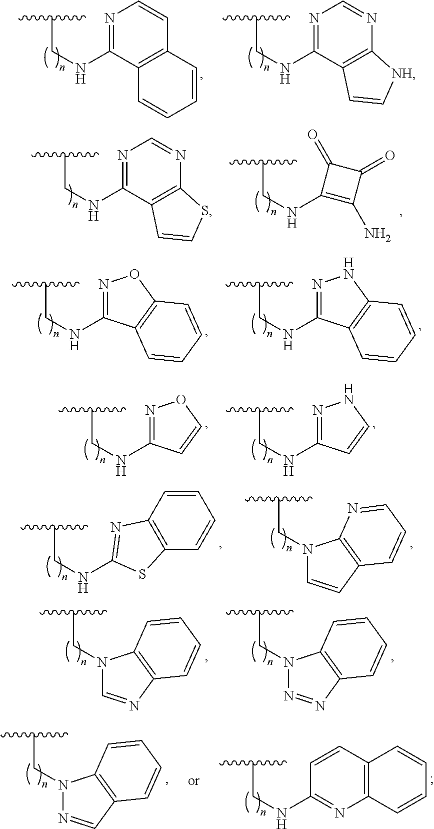
R² is H, optionally substituted alkyl, optionally substituted alkenyl, optionally substituted alkynyl, optionally substituted aryl, optionally substituted heteroaryl, hydroxy, optionally substituted alkoxy, optionally substituted amino, or halo,
R³ is H, optionally substituted alkyl, optionally substituted alkenyl, optionally substituted alkynyl, optionally substituted aryl, optionally substituted heteroaryl, hydroxy, optionally substituted alkoxy, optionally substituted amino, or halo;
R⁴ is H, optionally substituted alkyl, optionally substituted alkenyl, optionally substituted alkynyl, optionally substituted aryl, optionally substituted heteroaryl, hydroxy, optionally substituted alkoxy, optionally substituted amino, or halo;
R⁵ is H, optionally substituted alkyl, optionally substituted alkenyl, optionally substituted alkynyl, optionally substituted aryl, optionally substituted heteroaryl, hydroxy, optionally substituted alkoxy, optionally substituted amino, or halo; and
n is 0, 1, 2, 3, 4, or 5;
(b) said compound of Formula I is: 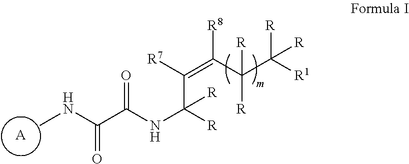
or a pharmaceutically acceptable salt or solvate thereof, wherein: 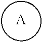
is optionally substituted aryl or heteroaryl;
R¹ is optionally substituted amino, 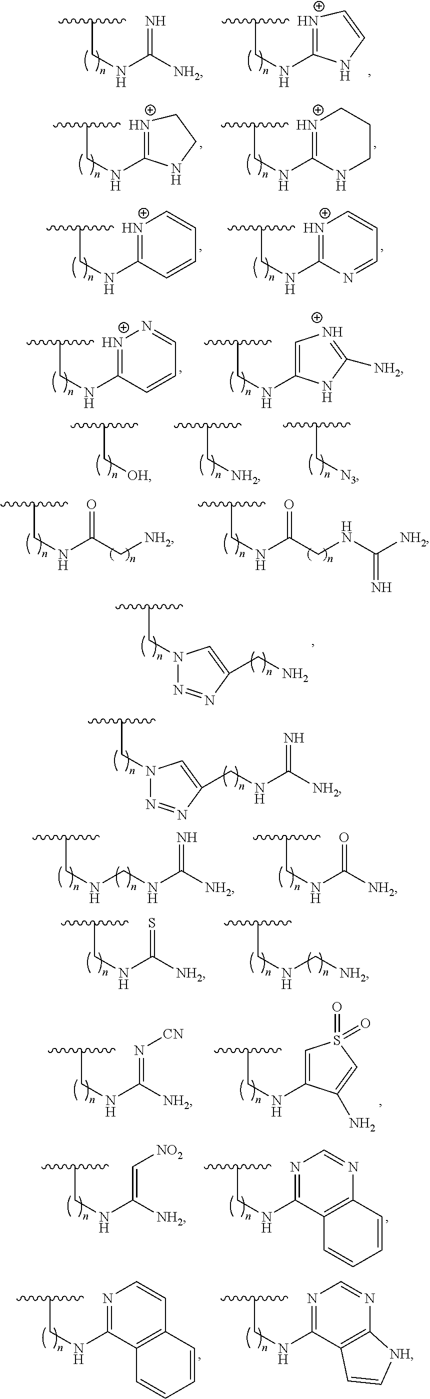 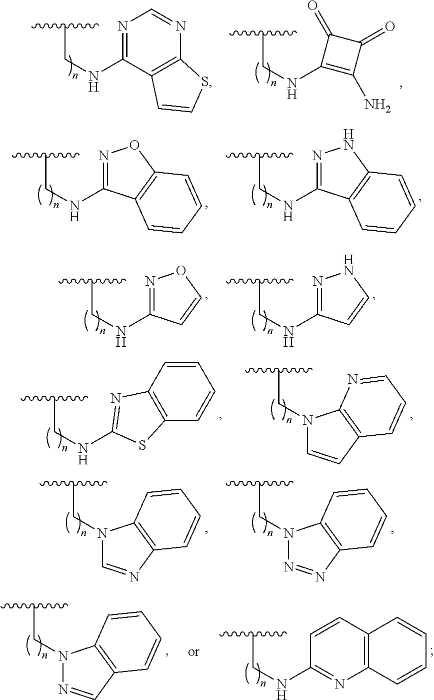
R⁷ is H optionally substituted alkyl, optionally substituted alkenyl optionally substituted alkynyl, optionally substituted aryl, optionally substituted heteroaryl, hydroxy, optionally substituted alkoxy, optionally substituted amino, or halo;
R⁸ is H, optionally substituted alkyl, optionally substituted alkenyl, optionally substituted alkynyl, optionally substituted aryl, optionally substituted heteroaryl, hydroxy, optionally substituted alkoxy, optionally substituted amino, or halo;
or, R⁷ and R⁸, taken together form an optionally substituted five-membered heteroaryl ring or an optionally substituted six-membered aryl or heteroaryl ring;
m is 1, 2, 3, or 4;
each R is independently H, optionally substituted alkyl, hydroxy, optionally substituted alkoxy, optionally substituted amino, or halo; and
n is 0, 1, 2, 3, 4, or 5;
(c) said compound of Formula II is: 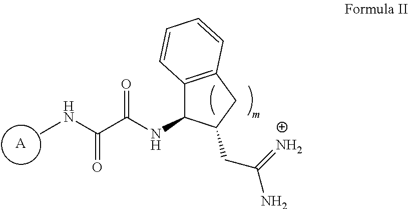
or a pharmaceutically acceptable salt or solvate thereof, wherein: 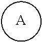
is optionally substituted aryl or optionally substituted heteroaryl; and
m is 1, 2, 3, or 4;
(d) said compound of Formula III is: 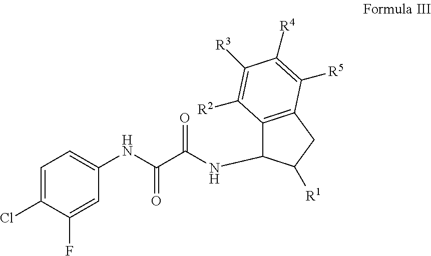
or a pharmaceutically acceptable salt or solvate thereof, wherein:
R¹ is optionally substituted amino, 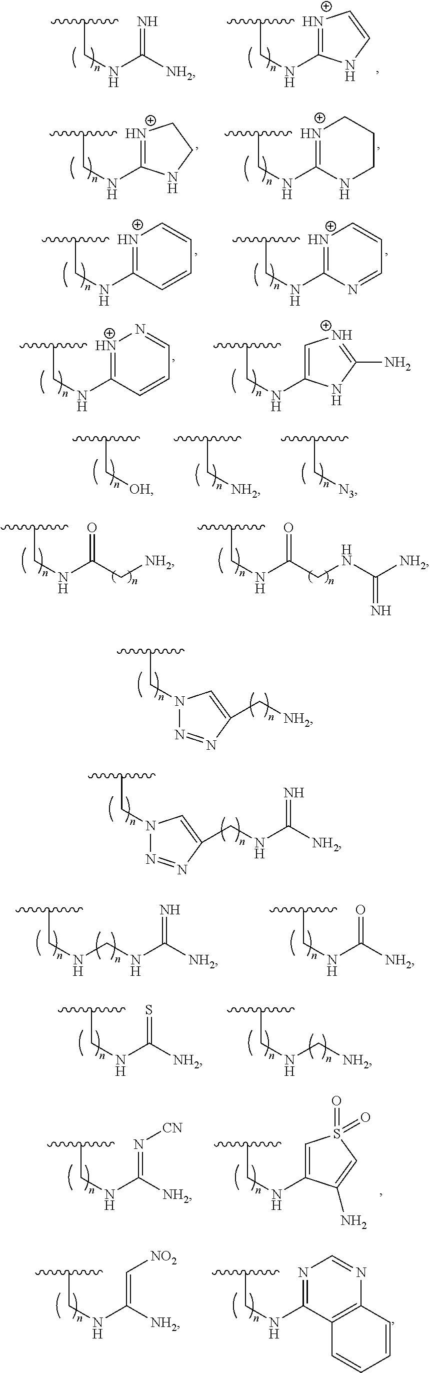 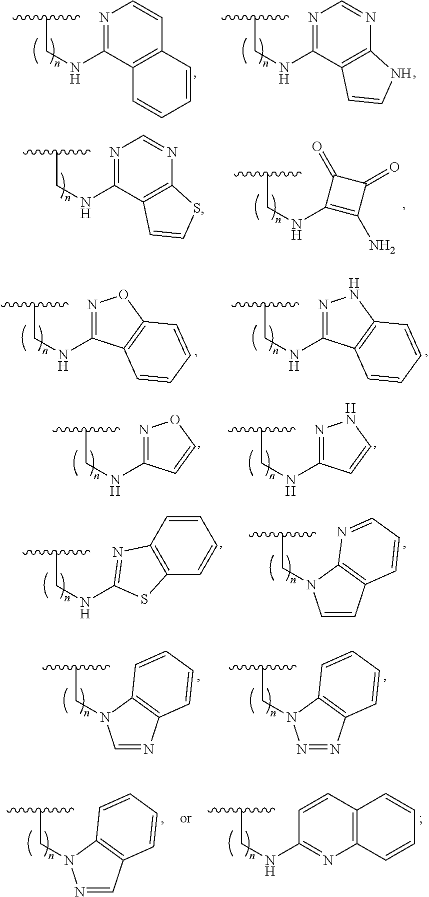
R² is H, optionally substituted alkyl, optionally substituted alkenyl, optionally substituted alkynyl, optionally substituted aryl, optionally substituted heteroaryl, hydroxy, optionally substituted alkoxy, optionally substituted amino, or halo;
R³ is H, optionally substituted alkyl, optionally substituted alkenyl, optionally substituted alkynyl, optionally substituted aryl, optionally substituted heteroaryl, hydroxy, optionally substituted alkoxy, optionally substituted amino, or halo;
R⁴ is H, optionally substituted alkyl, optionally substituted alkenyl, optionally substituted alkynyl, optionally substituted aryl, optionally substituted heteroaryl, hydroxy, optionally substituted alkoxy, optionally substituted amino, or halo;
R⁵ is H, optionally substituted alkyl, optionally substituted alkenyl, optionally substituted alkynyl, optionally substituted aryl, optionally substituted heteroaryl, hydroxy, optionally substituted alkoxy, optionally substituted amino, or halo; and
n is 0, 1, 2, 3, 4, or 5;
(e) said compound of Formula IV is: 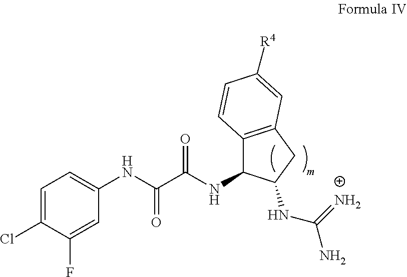
or a pharmaceutically acceptable salt or solvate thereof, wherein:
R⁴ is halo, hydroxy, thio, optionally substituted alkylsulfonamido, optionally substituted cycloalkylsulfonamido, optionally substituted amino, optionally substituted amido, optionally substituted heterocyclyl, optionally substituted heteroaryl, or optionally substituted aryl; and
m is 1, 2, 3, or 4;
(f) said compound of Formula V is: 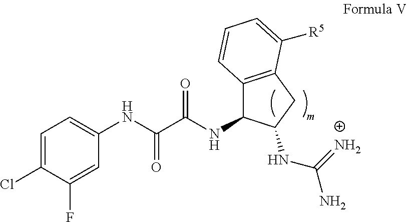
or a pharmaceutically acceptable salt or solvate thereof, wherein:
R⁵ is halo, hydroxy, thio, optionally substituted alkylsulfonamido, optionally substituted cycloalkylsulfonamido, optionally substituted amino, optionally substituted amido, optionally substituted heterocyclyl, optionally substituted heteroaryl, or optionally substituted aryl; and
m is 1, 2, 3, or 4:
(g) said compound of Formula VI is: 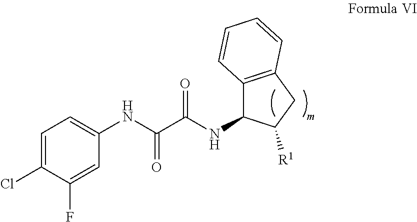
or a pharmaceutically acceptable salt or solvate thereof, wherein:
R¹ is optionally substituted amino, 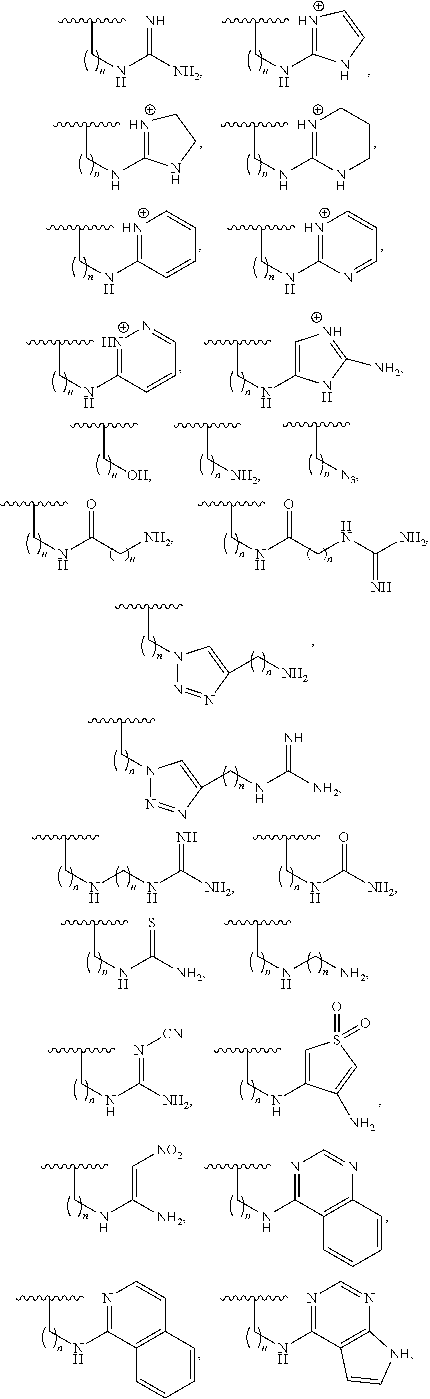 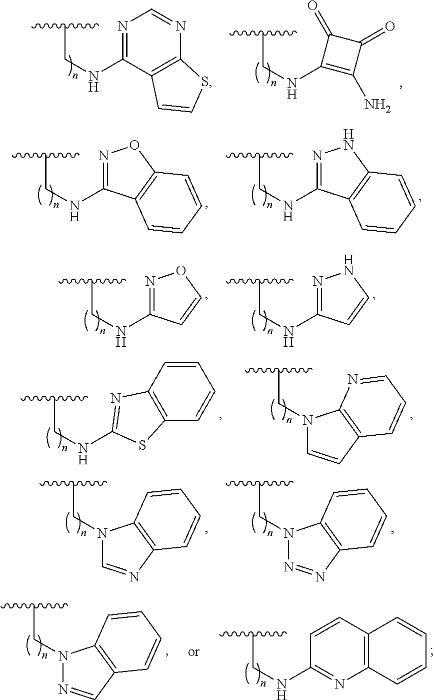
m is 1, 2, 3, or 4; and
n is 0, 1, 2, 3, 4, or 5; or
(h) said compound of Formula VIII is: 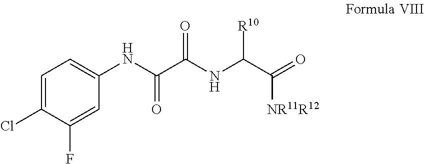
or a pharmaceutically acceptable salt or solvate thereof, wherein:
R¹⁰ is optionally substituted aryl, optionally substituted heteroaryl, optionally substituted cycloalkyl, optionally substituted heterocycloalkyl, optionally substituted alkyl, or optionally substituted alkenyl;
R¹¹ is H, optionally substituted alkyl, optionally substituted alkenyl, optionally substituted alkynyl, optionally substituted aryl, or optionally substituted heteroaryl; and
R¹² is optionally substituted alkyl, optionally substituted alkenyl, optionally substituted alkynyl, optionally substituted aryl, or optionally substituted heteroaryl.

13. The complex of claim 12, wherein said complex is in a solubilized form or immobilized to a solid support.
